(19) United States
(12) Patent Application Publication
(10) Pub. No.: US 2012/0021421 A1
(43) Pub. Date: Jan. 26, 2012

(54) Bacterial DNA as Markers of Cardiovascular and/or Metabolic Disease

(76) Inventors: Jacques Amar, Toulouse (FR); Remy Burcelin, Toulouse (FR); Chantal Chabo, Toulouse (FR)

(21) Appl. No.: 13/201,307

(22) PCT Filed: Feb. 12, 2010
(86) PCT No.: PCT/EP2010/051820

§ 371 Date: Oct. 6, 2011

(30) Foreign Application Priority Data
Feb. 13, 2009 (EP) 09305138.1

(51) Int. Cl. C12Q 1/68; C07H 21/04

(52) U.S. Cl. 435/6.12; 536/24.33

(57) ABSTRACT

The present invention relates to an in vitro method for predicting and/or diagnosing a cardiovascular and/or metabolic disease in a subject, which method comprises determining the concentration of bacterial DNA in a biological sample of said subject, together with degenerated primers for predicting and/or diagnosing a cardiovascular and/or metabolic disease in a subject.

The present invention relates to a method for predicting and/or diagnosing a cardiovascular or metabolic disease in a subject.

Metabolic diseases such as type 2 diabetes or obesity and their vascular complications are a major public health issue, the importance of which should increase with the spread of the obesity epidemic. The set-up of a low tonus but chronic metabolic-type inflammatory reaction is one of the cellular mechanisms that appear early during the development of these diseases. The active molecules which are released by the inflammatory process lower the insulin action and favour the development of fat tissue and atheroma plaques.

A growing body of evidence supports a link between digestive microflora, metabolic diseases and atherosclerosis. From an epidemiological point of view, large prospective observational studies showed that chronic infectious diseases involving Gram-negative bacteria such as periodontis predict the incidence of acute cardiovascular events (Howell et al. (2001) J. Am. Coll. Cardiol. 37:445-450, Dietrich et al. (2008) Circulation 117:1668-1674). Recently, a large population based study established the predictive value of periodontis on diabetes. Furthermore, many cross-sectional trials have reported the presence of various bacterial pathogens in the arterial vessel wall.

Accordingly, it has been hypothesized that the presence of bacteria could predict the occurrence of a metabolic and/or cardiovascular disease. Diagnosis methods of metabolic and cardiovascular diseases comprising the detection of bacteria have thus been proposed. Nevertheless, the only available methods need to investigate the presence of each bacterial strain potentially involved in the genesis of metabolic or cardiovascular diseases. Such methods are consequently costly and difficult to be performed in routine and in a large scale.

There is thus a need for new methods and new markers for predicting metabolic and/or cardiovascular diseases, which could be used routinely, in a large scale and at a low cost.

The present inventors have demonstrated that change in bacterial lipopolysaccharides (LPS) blood concentration, the main components of the outer wall of Gram-negative bacteria and the natural ligands of Toll-like receptor 4 which has been identified as central in the atherosclerosis process, was a causative triggering factor of weight intake and diabetes (Cani et al. (2007) Diabetes 56:1761-1772, Cani et al. (2008) Diabetes 57:1470-1481). From these results, they hypothesized that bacterial LPS could play a key role in humans on the atherosclerotic process and the development of metabolic diseases. Nevertheless, blood LPS concentration was not considered as a good candidate as marker of metabolic and/or cardiovascular diseases because 1) LPS was difficultly assayable in a blood sample; 2) its blood concentration could vary rapidly according to the feeding status and 3) no significant association between plasma LPS concentration and metabolic parameters of atherosclerotic plaques could be observed in a french population-based sample. Furthermore, no significant association between plasma LPS concentration in human and cardiovascular events has been reported so far.

The present invention arises from the unexpected finding, by the inventors, that the concentration of bacterial DNA in the blood of a subject is a reliable risk marker and a predictor of metabolic and/or cardiovascular diseases.

Thus, the present invention relates to an in vitro method for predicting and/or diagnosing a cardiovascular and/or metabolic disease in a subject, which method comprises determining the concentration of bacterial DNA in a biological sample of said subject.

### Cardiovascular and Metabolic Diseases

In the context of the invention, a “cardiovascular disease” refers to a disease that involves the heart or blood vessels (arteries and veins). More particularly, a cardiovascular disease according to the invention denotes a disease, lesion or symptom associated with an atherogenesis process that affects the cardiovascular system. It includes especially the conditions in which an atheroma plaque develops as well as the complications due to the formation of an atheroma plaque (stenosis, ischemia) and/or due to its evolution toward an acute ischemic stroke (thrombosis, embolism, infarction, arterial rupture).

Cardiovascular diseases include coronary artery disease, coronary heart disease, hypertension, atherosclerosis, in particular iliac or femoral atherosclerosis, angina pectoris, thrombosis, heart failure, stroke, vascular aneurysm, vascular calcification, myocardial infarction, vascular stenosis and infarction, and vascular dementia. Preferably, the cardiovascular disease according to the invention is selected from the group consisting in coronary artery disease, hypertension, atherosclerosis, vascular aneurysm, vascular calcification, vascular dementia and heart failure. More preferably, the cardiovascular disease according to the invention is atherosclerosis. Most preferably, the cardiovascular disease according to the invention is atherosclerotic carotid plaques.

As used herein, a “coronary artery disease” denotes a disease corresponding to the end result of the accumulation of atheromatous plaques within the walls of the arteries that supply the myocardium with oxygen and nutrients. It is one of the most common causes of coronary heart disease.

As used herein, a “coronary heart disease” denotes a progressive disease, due to a bad irrigation of the heart muscle, consecutive to the narrowing (stenosis) or calcification (sclerosis) of a coronary artery. The complete obstruction of a coronary artery leads to myocardial infarction.

As used herein, “hypertension”, also referred to as “high blood pressure”, “HTN” or “HPN”, denotes a medical condition in which the blood pressure is chronically elevated.

As used herein, “atherosclerosis” denotes a disease affecting arterial blood vessels. Atherosclerosis can be characterized by a chronic inflammatory response in the walls of arteries, mainly due to the accumulation of macrophages and promoted by low density lipoproteins without adequate removal of fats and cholesterol from macrophages by functional high density lipoproteins.

As used herein, an “atheroma plaque” or “atherosclerotic plaque” refers to a lesion of vessel walls. Preferably, an “atherosclerotic plaque” comprises a lipid core and a fibrous cap, said cap being constituted by smooth muscle cells, collagens and an extracellular matrix and isolating the lipid core from the arterial lumen. Atherosclerotic plaques may be found for example in the aorta, the carotid, or in the coronary artery. When the plaque comprises a thin fibrous cap (about 65 to 150 μm thick) and a considerable lipid core, they are referred to as “vulnerable atherosclerotic plaques” or “vulnerable atheroma plaques” or “vulnerable plaques”. These plaques, which are prone to rupture, may be found in coronary arteries and in aorta and its branches.

As used herein, “angina pectoris” or “angina” denotes a severe chest pain due to ischemia of the heart muscle.

As used herein, “thrombosis” denotes the formation of a blood clot inside a blood vessel.

As used herein, “heart failure” denotes a condition in which a problem with the structure or function of the heart impairs its ability to supply sufficient blood flow to meet the body's needs.

As used herein, a “stroke” denotes the rapidly developing loss of brain functions due to a disturbance in the blood vessels supplying blood to the brain.

As used herein, a “vascular aneurysm” or “aneurysm” denotes a localized, blood-filled dilation of a blood vessel caused by a disease or weakening of the vessel wall. Aneurysms most commonly occur in arteries at the base of the brain and in the aorta.

As used herein, an “infarction” denotes the process resulting in a macroscopic area of necrotic tissue in some organ caused by loss of adequate blood supply. In particular, a “myocardial infarction” denotes the interruption of blood supply to part of the heart.

As used herein, a “vascular dementia” denotes a degenerative cerebrovascular disease that leads to a progressive decline in memory and cognitive functioning. It includes in particular multi-infarct dementia (multiple large and complete infarcts), post-hemorrhage dementia, subcortical vascular dementia (small vessel disease) and mixed demention (combination of Alzheimer disease and vascular dementia).

In the context of the invention, a “metabolic disease” denotes a disease that disrupts normal metabolism. Preferably, the metabolic disease according to the invention is a carbohydrate metabolism disorder.

As used herein a “carbohydrate metabolism disorder” denotes a disease wherein the metabolism of carbohydrate, for example of glucose, is disrupted. Carbohydrate metabolism disorders include diabetes, high fasting glycemia, overweight and obesity.

As used herein, “diabetes” denotes a syndrome of disordered metabolism, usually due to a combination of hereditary and environmental causes, resulting in abnormally high blood sugar levels.

As used herein, “high fasting glycemia” denotes a syndrome of disordered metabolism, resulting in a glycemia of more than 6.1 mmol/l.

As used herein, “overweight” denotes a condition in which excess body fat has accumulated to such an extent that health may be negatively affected. Overweight is commonly defined as a body mass index of 25 kg/m² or higher.

As used herein, “obesity” denotes a condition in which excess body fat has accumulated to such an extent that health may be negatively affected. Obesity is commonly defined as a body mass index of 30 kg/m² or higher.

### Bacterial DNA

In the context of the invention, “bacterial DNA” refers to a DNA belonging to any bacteria. In particular, a bacterial DNA according to the invention denotes a DNA sequence from the genome of a bacterium.

Preferably, the bacterial DNA is 16S DNA and/or LpxB DNA.

As used herein, “16S DNA” refers to the gene encoding the 16S ribosomal RNA (16S rRNA) constituted of about 1500 nucleotides, which is the main component of the small prokaryotic ribosomal subunit (30S). 16S DNA is highly conserved among bacteria. The reference Escherichia coli 16S rRNA gene sequence corresponds to SEQ ID NO: 1745 (called rrsA). In the context of the invention, 16S DNA refers to any sequence corresponding to SEQ ID NO: 1745 in other bacterial strains.

As used herein, “LpxB DNA” refers to the bacterial LpxB gene. The LpxB gene refers to a bacterial gene encoding the lipid A disaccharide synthase that catalyses the condensation of 2,3-bis(3-hydroxymyristol)-β-D-glucosaminyl 1-phosphate and UDP-2,3-bis(3-hydroxymyristoyl)glucosamine (lipid X) to form lipid A disaccharide. This reaction represents one of the first enzymatic reactions involved in lipid A biosynthesis. The reference Escherichia coli LpxB DNA sequence corresponds to SEQ ID NO: 1746. In the context of the invention, LpxB DNA refers to any sequence corresponding to SEQ ID NO: 1746 in other bacterial strains.

### Method for in vitro Predicting and/or Diagnosing

The present invention relates to an in vitro method for predicting and/or diagnosing a cardiovascular and/or metabolic disease in a subject, which method comprises determining the concentration of bacterial DNA in a biological sample of said subject.

As used herein, a “diagnosing method” or “diagnostic method” or “diagnosis” refers to a method for determining whether an individual suffers from a disease.

As used herein, a “predicting method” refers to a method for determining whether an individual is likely to develop a disease.

In the context of the present invention, a “subject” denotes a human or non-human mammal, such as a rodent (rat, mouse, rabbit), a primate (chimpanzee), a feline (cat), a canine (dog). Preferably, the subject is human.

As used herein, the term “biological sample” means a substance of biological origin. Examples of biological samples include, but are not limited to, blood and components thereof such as serum, plasma, platelets, subpopulations of blood cells such as leucocytes; urine, and tissues such as adipose tissues, hepatic tissues and the like. Preferably, a biological sample according to the present invention is a blood, serum, plasma, leucocytes, urine, adipose tissue or hepatic tissue sample.

In a particular embodiment, the method as defined above is for predicting and/or diagnosing a cardiovascular and metabolic disease in a subject and comprises determining the bacterial 16S DNA concentration and the LpxB DNA concentration in the biological sample of said subject.

In another particular embodiment, the method as defined above is for predicting and/or diagnosing a cardiovascular disease in a subject and comprises determining the LpxB DNA concentration in the biological sample of said subject.

In still another particular embodiment, the method as defined above is for predicting and/or diagnosing a metabolic disease in a subject and comprises determining the bacterial 16S DNA concentration in the biological sample of said subject. Preferably, this method further comprises determining LpxB DNA concentration in the biological sample of said subject.

Preferably, the bacterial DNA concentration is measured by polymerase chain reaction (PCR), more preferably by quantitative PCR (qPCR), most preferably by real-time quantitative PCR (RT-qPCR).

As used herein, “real-time quantitative PCR”, “real-time polymerase chain reaction”, or “kinetic polymerase chain reaction” refers to a laboratory technique based on the polymerase chain reaction, which is used to amplify and simultaneously quantify a targeted DNA molecule. It enables both detection and quantification (as absolute number of copies or relative amount when normalized to DNA input or additional normalizing genes) of a specific sequence in a sample. Two common methods of quantification are the use of fluorescent dyes that intercalate with double-stranded DNA, and modified DNA oligonucleotide probes that fluoresce when hybridized with a complementary DNA.

The present inventors specifically designed a method enabling the accurate determination of the bacterial 16S DNA concentration in a biological sample of a subject. In particular, this method enables discriminating bacterial 16S DNA and mitochondrial DNA.

The invention therefore also relates to an in vitro method for determining the bacterial 16S DNA concentration in a biological sample of a subject comprising the steps consisting of:
a) amplifying, preferably by real time PCR, known quantities of bacterial 16S DNA and mitochondrial DNA using universal primers;
b) defining the melting temperature of bacterial 16S DNA and mitochondrial DNA;
c) establishing a standard curve representing the ratio of the negative first-derivatives of the normalized fluorescence (−Rn′) measured for bacterial 16S DNA and mitochondrial DNA according to the Log 10 of the quantity of mitochondrial DNA/quantity of bacterial 16S DNA ratio;
d) deducing therefrom the values of the constants “Bottom”, “Top”, “Log EC50” and “HillSlope” in the following equation

Y=Bottom+(Top−Bottom)/[1+10̂((Log EC50−X)*HillSlope)]

wherein

X=Log 10(mitochondrial DNA/bacterial 16S DNA), and

Y=−Rn′ read at the melting temperature of mitochondrial DNA/−Rn′ read at the melting temperature of bacterial 16S DNA;
e) amplifying DNA of the biological sample using the universal primers;
f) defining the melting temperature of the DNA of the biological sample; and
g) deducing therefrom the bacterial 16S DNA concentration in the biological sample.

As used herein, the term “amplifying” or “amplification” refers to the multiplication of a DNA template. Preferably, the amplification of step a) is performed by PCR, more preferably by quantitative PCR, most preferably by real-time quantitative PCR. Preferably, when the amplification is performed by PCR, a reporter that produces fluorescence, such as SYBR Green, is used.

In the context of the invention, the term “universal primers” refers to primers comprising a sequence which is able to hybridize to 16S DNA from essentially any origin. Preferably, the universal primers according to the invention are primers comprising a sequence selected from the group consisting of the sequence 5′-ACTCCTACGGGAGGCAGCAGT-3′ (SEQ ID NO: 1748) and the sequence 5′-GTATTACCGCGGCTGCTGGCAC-3′ (SEQ ID NO: 1749).

As used herein, the term “melting temperature” refers to the temperature wherein the DNA double strands dissociate. As known from the person skilled in the art, the melting temperature depends on the GC content of the DNA. The present inventors have demonstrated that the melting temperature of mitochondrial DNA was 75.5° C. whereas the melting temperature of bacterial 16S DNA was 82° C., which enables discriminating both types of DNA.

As used herein, the expression “standard curve” refers to a method of plotting assay data that is used to determine the concentration of a substance and which is first performed with various known concentrations of a substance similar to that being measured.

In the context of the invention, the term “Bottom” refers to the lower value of the standard curve.

As used herein, the term “Top” refers to the maximal value of the standard curve.

As used herein, the term “Log EC50” refers to the Log 10 value of the quantity of mitochondrial DNA/quantity of bacterial 16S DNA ratio that gives a −Rn′ half way between bottom and top.

As used herein, the term “HillSlope” describes the steepness of the family of response curves.

### Nucleic Acids, Primers and Pairs of Primers, Probes

The present invention also relates to an isolated nucleic acid which comprises a sequence that hybridizes specifically under high stringency conditions to LpxB DNA or bacterial 16S DNA and to the use, preferably in vitro, of at least one isolated nucleic acid which comprises a sequence that hybridizes specifically under high stringency conditions to LpxB DNA or bacterial 16S DNA for detecting the presence of bacteria in a biological sample of a subject.

As used herein, the term “isolated nucleic acid” refers to both RNA and DNA, including cDNA, genomic DNA, and synthetic DNA. Nucleic acids can have any three-dimensional structure. A nucleic acid can be double-stranded or single-stranded (i.e., a sense strand or an antisense strand). Non-limiting examples of nucleic acids include genes, gene fragments, exons, introns, messenger RNA (mRNA), transfer RNA (tRNA), ribosomal RNA, siRNA, micro-RNA, ribozymes, cDNA, recombinant nucleic acids, and branched nucleic acids. A nucleic acid may contain unconventional or modified nucleotides. Isolated nucleic acids according to the invention may be purified or recombinant.

In the context of the invention, the terms “hybridize” or “hybridization,” as is known to those skilled in the art, refer to the binding of a nucleic acid molecule to a particular nucleotide sequence under suitable conditions, namely under stringent conditions.

The term “stringent conditions” or “high stringency conditions” as used herein corresponds to conditions that are suitable to produce binding pairs between nucleic acids having a determined level of complementarity, while being unsuitable to the formation of binding pairs between nucleic acids displaying a complementarity inferior to said determined level. Stringent conditions are the combination of both hybridization and wash conditions and are sequence dependent. These conditions may be modified according to methods known from those skilled in the art (Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). Generally, high stringency conditions are selected to be about 5° C. lower than the thermal melting point (Tm), preferably at a temperature close to the Tm of perfectly base-paired duplexes (Andersen, Nucleic acid Hybridization, Springer, 1999, p. 54). Hybridization procedures are well known in the art and are described for example in Ausubel, F. M., Brent, R., Kingston, R. E., Moore, D. D., Seidman, J. G., Smith, J. A., Struhl, K. eds. (1998) Current protocols in molecular biology. V. B. Chanda, series ed. New York: John Wiley & Sons.

High stringency conditions typically involve hybridizing at about 50° C. to about 68° C. in 5×SSC/5× Denhardt's solution/1.0% SDS, and washing in 0.2×SSC/0.1% SDS at about 60° C. to about 68° C.

As used herein, the expression “hybridizes specifically” indicates that the nucleic acid of a determined sequence displays a sufficient degree of complementarity with a target sequence to form a stable binding between the nucleic acid and the target sequence and to avoid non-specific binding with a non-target sequence, under high stringency conditions. The degree of complementarity is calculated by comparing the sequence of said nucleic acid optimally aligned with the complementary target sequence, determining the number of positions at which the nucleic acid bases are complementary to yield the number of complementary positions, dividing the number of complementary positions by the total number of positions of the target sequence, and multiplying the result by 100 to yield the degree of complementarity. The degree of complementarity may be determined routinely using BLAST programs (basic local alignment search tools) and PowerBLAST programs known in the art (Altschul et al., (1990) J. Mol. Biol. 215: 403-410; Zhang and Madden (1997) Genome Res. 7:649-656).

In a particular embodiment, the at least one isolated nucleic acid as defined above is used, preferably in vitro, for predicting and/or diagnosing a metabolic and/or cardiovascular disease in a subject.

In a preferred embodiment, said nucleic acid is a probe or a primer.

As used herein, a “probe” refers to an oligonucleotide capable of binding in a base-specific manner to a complementary strand of nucleic acid. Probes according to the invention may be purified or recombinant. They may be labelled with a detectable moiety, i.e. a moiety capable of generating a detectable signal, such as radioactive, calorimetric, fluorescent, chemiluminescent or electrochemiluminescent signal. Numerous such detectable moieties are known in the art. By way of example, the moiety may be a radioactive compound or a detectable enzyme (e.g. horseradish peroxidase (HRP)). Preferably, the probe according to the invention comprises or is constituted of from about 10 to about 1000 nucleotides.

As used herein, the term “primer” refers to an oligonucleotide which is capable of annealing to a target sequence and serving as a point of initiation of DNA synthesis under conditions suitable for amplification of the primer extension product which is complementary to said target sequence. The primer is preferably single stranded for maximum efficiency in amplification. Preferably, the primer is an oligodeoxyribonucleotide. The length of the primer depends on several factors, including temperature and sequence of the primer, but must be long enough to initiate the synthesis of amplification products. Preferably the primer is from 10 to 35 nucleotides in length. More preferably, the primer is from 15 to 30 nucleotides in length. Most preferably, the primer is 22 or 29 nucleotides in length. A primer can further contain additional features which allow for detection, immobilization, or manipulation of the amplified product. The primer may furthermore comprise covalently-bound fluorescent dyes, which confer specific fluorescence properties to the hybrid consisting of the primer and the target sequence or non covalently-bound fluorescent dyes which can interact with the double-stranded DNA/RNA to change the fluorescence properties. Fluorescent dyes which can be used are for example SYBR-green or ethidium bromide.

The present inventors have designed degenerated primers able to amplify the LpxB gene in all bacterial genomes currently registered in NCBI database, in a region corresponding to nucleotides 154 to 299 in Escherichia coli. These primers border a region of 146 nucleotides which amplifies various DNA fragments corresponding to LpxB genes.

Accordingly, the present invention also relates to an isolated nucleic acid which comprises a sequence selected from the group consisting of the sequence 5′-GAAGCNTGGTANGANATGGAAG-3′ (SEQ ID NO: 1), wherein N in 6^{th} position from 5′ is 90% C or 10% T, N in 12^{th} position from 5′ is 60% C or 40% T and N in 15^{th} position from 5′ is 60% A or 40% G, and the sequence 5′-GGNGCNTCNATNCCNACNAANACATCNGG-3′ (SEQ ID NO: 10), wherein N in 3^{rd} position from 5′ is 50% C or 50% G, N in 6^{th} position from 5′ is 70% C or 30% T, N in 9^{th} position from 5′ is 90% A or 10% G, N in 12^{th} position from 5′ is 50% A, 15% C or 35% T, N in 15^{th} position from 5′ is 60% A, 10% C or 30% G, N in 18^{th} position from 5′ is 90% A or 10% G, N in 21^{st} position from 5′ is 45% A, 10% C or 45% G and N in 27^{th} position from 5′ is 10% A, 10% C, 40% G or 40% T. In other words, when the isolated nucleic acid as defined above comprises the sequence 5′-GAAGCNTGGTANGANATGGAAG-3′ (SEQ ID NO: 1), wherein N in 6^{th} position from 5′ is 90% C or 10% T, N in 12^{th} position from 5′ is 60% C or 40% T and N in 15^{th} position from 5′ is 60% A or 40% G, said isolated nucleic acid comprises a sequence selected from the group consisting of SEQ ID NO: 2 to SEQ ID NO: 9. Similarly, when the isolated nucleic acid as defined above comprises the sequence 5′-GGNGCNTCNATNCCNACNAANACATCNGG-3′ (SEQ ID NO: 10), wherein N in 3^{rd} position from 5′ is 50% C or 50% G, N in 6^{th} position from 5′ is 70% C or 30% T, N in 9^{th} position from 5′ is 90% A or 10% G, N in 12^{th} position from 5′ is 50% A, 15% C or 35% T, N in 15^{th} position from 5′ is 60% A, 10% C or 30% G, N in 18^{th} position from 5′ is 90% A or 10% G, N in 21^{st} position from 5′ is 45% A, 10% C or 45% G and N in 27^{th} position from 5′ is 10% A, 10% C, 40% G or 40% T, said isolated nucleic acid comprises a sequence selected from the group consisting of SEQ ID NO: 11 to SEQ ID NO: 1738.

As used herein, the expression “wherein N is x % X or y % Y” means that the nucleotide N is X in x % of the cases and Y in y % of the cases.

The present invention also encompasses the use, preferably in vitro, of the isolated nucleic acid as defined above for predicting and/or diagnosing a metabolic and/or cardiovascular disease in a subject. It also relates to the isolated nucleic acid as defined above for use for predicting and/or diagnosing a metabolic and/or cardiovascular disease in a subject.

The present invention also relates to an in vitro method for predicting and/or diagnosing a metabolic and/or cardiovascular disease in a subject, wherein a nucleic acid as defined above is used.

The present invention also relates to an isolated primer comprising the nucleic acid as defined above.

Another object of the present invention relates to a pair of primers comprising a first and a second primer, wherein the first primer comprises a nucleic acid which consists of the sequence 5′-GAAGCNTGGTANGANATGGAAG-3′ (SEQ ID NO: 1), wherein N in 6^{th} position from 5′ is 90% C or 10% T, N in 12^{th} position from 5′ is 60% C or 40% T and N in 15^{th} position from 5′ is 60% A or 40% G; and the second primer comprises a nucleic acid which consists of the sequence 5′-GGNGCNTCNATNCCNACNAANACATCNGG-3′ (SEQ ID NO: 10), wherein N in 3^{rd} position from 5′ is 50% C or 50% G, N in 6^{th} position from 5′ is 70% C or 30% T, N in 9^{th} position from 5′ is 90% A or 10% G, N in 12^{th} position from 5′ is 50% A, 15% C or 35% T, N in 15^{th} position from 5′ is 60% A, 10% C or 30% G, N in 18^{th} position from 5′ is 90% A or 10% G, N in 21′ position from 5′ is 45% A, 10% C or 45% G and N in 27^{th} position from 5′ is 10% A, 10% C, 40% G or 40% T. In other words, the first primer of the pair of primers according to the invention comprises a nucleic acid which consists of a sequence selected from the group consisting of SEQ ID NO: 2 to SEQ ID NO: 9, and the second primer of the pair of primers according to the invention comprises a nucleic acid which consists of a sequence selected from the group consisting of SEQ ID NO: 11 to SEQ ID NO: 1738.

Another object of the present invention relates to the use, preferably in vitro, of at least one nucleic acid as defined above wherein said at least one nucleic acid is a primer of sequence as defined above or a primer from a pair of primers of sequences as defined above.

The present invention also relates to an in vitro method for predicting and/or diagnosing a metabolic and/or cardiovascular disease in a subject, wherein a primer or a pair of primers as defined above is used.

The present inventors have also designed primers which are specific for 16S DNA. In particular, these primers do not cross hybridize with mitochondrial DNA.

The present invention therefore also relates to an isolated nucleic acid which comprises a sequence selected from the group consisting of the sequence 5′-TCCTACGGGAGGCAGCAGT-3′ (SEQ ID NO: 1750) and the sequence 5′-GGACTACCAGGGTATCTAATCCTGTT-3′ (SEQ ID NO: 1751).

The present invention also encompasses the use, preferably in vitro, of the nucleic acid as defined above for predicting and/or diagnosing a metabolic and/or cardiovascular disease in a subject. It also relates to the isolated nucleic acid as defined above for use for predicting and/or diagnosing a metabolic and/or cardiovascular disease in a subject.

The present invention also relates to an in vitro method for predicting and/or diagnosing a metabolic and/or cardiovascular disease in a subject, wherein a nucleic acid as defined above is used.

The present invention also relates to an isolated primer comprising the nucleic acid as defined above.

Another object of the present invention relates to a pair of primers comprising a first and a second primer, wherein the first primer comprises a nucleic acid which consists of the sequence 5′-TCCTACGGGAGGCAGCAGT-3′ (SEQ ID NO: 1750) and the second primer comprises a nucleic acid which consists of the sequence 5′-GGACTACCAGGGTATCTAATCCTGTT-3′ (SEQ ID NO: 1751).

Another object of the present invention relates to the use, preferably in vitro, of at least one nucleic acid as defined above wherein said at least one nucleic acid is a primer of sequence as defined above or a primer from a pair of primers of sequences as defined above.

The present invention also relates to an in vitro method for predicting and/or diagnosing a metabolic and/or cardiovascular disease in a subject, wherein a primer or a pair of primers as defined above is used.

### Kits

Another object of the present invention relates to a kit for predicting and/or diagnosing a metabolic and/or cardiovascular disease in a subject, wherein said kit comprises at least one nucleic acid that hybridizes specifically under high stringency conditions to LpxB DNA, and optionally instructions for use.

Preferably, said kit further comprises at least one nucleic acid that hybridizes under high stringency conditions to bacterial 16S DNA. Examples of suitable nucleic acids include in particular nucleic acids hybridizing to the conserved region of the gene coding for bacterial 16S rRNA and bordering the variable V3 region.

More preferably, said kit comprises at least one primer of sequence as defined above or at least one pair of primers of sequences as defined above. Most preferably, the kit comprises the primers of sequence SEQ ID NO: 2 to SEQ ID NO: 9 and SEQ ID NO: 11 to SEQ ID NO: 1738.

Preferably, said kit further comprises at least one nucleic acid which comprises a sequence selected from the group consisting of the sequence 5′-TCCTACGGGAGGCAGCAGT-3′ (SEQ ID NO: 1750) and the sequence 5′-GGACTACCAGGGTATCTAATCCTGTT-3′ (SEQ ID NO: 1751).

“Instructions for use” typically include a tangible expression describing the technique to be employed in using the components of the kit and explaining how to correlate detection of LpxB DNA, and optionally of bacterial 16S DNA, to the prediction or diagnosis of a metabolic and/or cardiovascular disease.

### Screening Methods

The present invention also relates to a method, preferably in vitro, for screening probiotics, prebiotics or chemical or biological compounds suitable for preventing and/or treating metabolic and/or cardiovascular diseases, comprising determining the bacterial 16S DNA concentration and/or LpxB DNA concentration in a biological sample of a subject which has been treated with the candidate probiotic, prebiotic or chemical or biological compound, and comparing said concentrations with those of a control subject which has not been treated. Specifically, a higher bacterial 16S DNA concentration and/or a higher LpxB DNA concentration in the biological sample of the control subject than in the biological sample of the subject treated with the candidate probiotic, prebiotic or chemical or biological compound, indicates said candidate probiotic, prebiotic or chemical or biological compound is a probiotic, prebiotic or chemical or biological compound suitable for preventing and/or treating metabolic and/or cardiovascular diseases.

As used herein, the term “probiotics” denotes dietary supplements and live microorganisms containing potentially beneficial bacteria or yeasts. According to the currently adopted definition by FAO/WHO, probiotics correspond to live microorganisms which when administered in adequate amounts confer a health benefit on the host. Examples of probiotics according to the invention include bifidobacteria, lactobacillus, bacteroids and fusobacteria.

As used herein, the term “prebiotics” denotes a non-digestible food ingredient that beneficially affects the host by selectively stimulating as a substrate the growth and/or activity of one or a limited number of bacteria in the colon, and thus improves host health.

In the context of the invention, “prebiotics” encompass isolated or purified prebiotics as well as natural prebiotics present in dietary supplements.

In the context of the invention, “probiotics”, encompass isolated or purified probiotics as well as natural probiotics present in dietary supplements.

As used herein, the term “chemical or biological compound” encompasses chemically synthetized compounds and compounds of biological origin which have an effect on the growth, metabolism, the survival of bacteria and/or their passage through the intestinal barrier. In particular, chemical or biological compounds according to the invention include molecules which modify the bacterial flora of the digestive tract and/or which modify the migration of bacteria through the digestive tract and/or which modify the permeability of the intestinal epithelial barrier. Examples of chemical or biological compounds of the invention include bactericides, antibiotics, as well as compounds acting on epithelial intercellular tight junctions, microvillies, cell coat, and/or intestinal epithelial cells.

The control subject which has not been treated may be a subject unrelated to the subject receiving the candidate prebiotic, probiotic or chemical or biological compound, or the same subject before treatment with the candidate prebiotic, probiotic or chemical or biological compound.

### Method for Identifying Predictive Markers of Cardiovascular and/or Metabolic Disease

The present invention also relates to an in vitro method for identifying predictive markers of a cardiovascular and/or metabolic disease, comprising

(i) detecting at least one sequence of bacterial DNA in a biological sample of an apparently healthy subject,

(ii) analyzing parameters that are indicative of a cardiovascular and/or metabolic disease in said subject and

(iii) identifying as predictive markers the sequences detected in the biological sample of said subject which are positively associated with the parameters that are indicative of a cardiovascular and/or metabolic disease.

As used herein, “apparently healthy” means subjects who have not previously had a cardiovascular event such as a myocardial infarction. Apparently healthy subjects also do not otherwise exhibit symptoms of disease. In other words, such subjects, if examined by a medical professional, would be characterized as healthy and free of symptoms of disease.

In the context of the invention, a “predictive marker of a cardiovascular and/or metabolic disease” refers to a marker that enables to make finding that a subject has a significantly enhanced probability of developing a cardiovascular and/or metabolic disease. As used herein, the marker is a sequence of bacterial DNA, preferably a gene.

Examples of parameters that are indicative of a cardiovascular and/or metabolic disease are well-known from those skilled in the art and include plasma total cholesterol, plasma total triglycerides, high-density lipoprotein (HDL) cholesterol, low-density lipoprotein (LDL) cholesterol, glycemia and C reactive protein (CRP).

As used herein, the expression “positively associated” means that there is a correlation between the predictive marker sequence and the parameters indicative of a cardiovascular and/or metabolic disease.

The term “correlation” indicates herein a statistical relationship between two random variables. The correlation between two variables may be determined by calculating the correlation coefficient between the variables. Statistical tests suitable to determine the correlation between two variables are well-known from those skilled in the art and include the Pearson product-moment correlation coefficient, which is obtained by dividing the covariance of the two variables by the product of their standard deviations, the Spearman's rank correlation coefficient or Spearman's rho, which is a special case of the Pearson product-moment coefficient in which two sets of data X, and Y, are converted to rankings x, and y, before calculating the coefficient.

The invention will be further illustrated by the following figures and examples.

### DESCRIPTION OF THE FIGURES

FIG. 1 shows a graph representing four dose response curves of the derivative reporters (−Rn′) ratio between eukaryotic and prokaryotic DNA read for 75.5° C. and 82° C. melting temperature respectively according to the Log 10 ratio between eukaryotic and prokaryotic DNA quantities. Increasing quantities of prokaryotic DNA were incubated with fixed quantities of eukaryotic DNA (0.1; 1; 10 and 100 ng).

FIG. 2 shows a graph representing a dose response curve of threshold cycle values (Ct) according to Log 10 ng prokaryotic DNA. Increasing quantities of prokaryotic DNA were incubated with a fixed quantity of eukaryotic DNA (0.1 ng).

FIG. 3 shows a graph representing the dose response curve of the derivative reporters (−Rn′) ratio between eukaryotic and prokaryotic DNA as a function of increasing Log 10 eukaryotic/prokaryotic quantities ratio. Increasing quantities of prokaryotic DNA were incubated with a fixed quantity of eukaryotic DNA (100 ng) used to establish the best fit formulae.

### EXAMPLE 1

The following example provides evidence showing that bacterial 16S DNA and LpxB DNA are potent markers of the presence of a metabolic and/or cardiovascular disease.

### Materials and Methods

### Population

Among 1015 subjects, aged between 35 and 64 years, randomly selected from the polling lists by the Toulouse MONICA centre between 1995 and 1997, 311 men with complete data for all the measurements, were analysed. Authorization from the appropriate Ethics Committee (Comité Consultatif de Protection des Personnes dans la Recherche Biomédicale, Lille) was obtained and each subject signed an informed consent.

### Questionnaire and Anthropometrical Measurements

The examination was performed in the morning and a blood sample was drawn after an overnight fast. Helped by the medical staff, each subject filled in a questionnaire on his/her medical history, drug intake, smoking habits, and alcohol consumption. Height, weight, waist and hip circumferences were measured. Tobacco consumption was defined according to current and past consumption.

### Blood Pressure (BP) and Resting Heart Rate Measurements

BP measurements were performed with a standard sphygmomanometer (Mercurex III, Spengler, Paris) using cuff size adapted to the subject's arm circumference. After a 5-min rest at least, carotid-femoral pulse wave velocity (PWV) was measured in supine position. Immediately after, heart rate and BP were measured twice in supine position. The average of the BP measurements was used for the statistical analysis.

### Aortic Stiffness

Aortic stiffness was assessed by carotid-femoral PWV measurement with a semi-automatic device Complior (Garge les Gonesse, France). The two pressure waveforms were digitised. Time delay (t) between the two pressure upstrokes was automatically calculated. This procedure was repeated on 10 cardiac cycles and the mean value was used for the statistical analysis. The distance covered by the pulse wave was measured on the surface of the body and represented the distance between the 2 recording sites (D). PWV was automatically calculated as PWV=D/t. For the sub-sample (n=26) of subjects submitted to a second measurement, the intra-class coefficient for PWV was 0.80 (p<0.001).

### Assessment of Carotid Plaques

High-resolution B-mode ultrasonography was used to detect atherosclerotic plaques in carotid arteries. An ATL UM9 system (Advanced Technology Laboratories Ultramark 9 High Definition Imaging) was used with a 7.4 MHz transducer. A plaque was defined as a distinct zone identified with either a focal area of hyperechogenicity relative to adjacent segments or a focal protrusion into the lumen of the vessel, composed of only calcified deposits or a combination of calcified and non calcified material. The presence of atherosclerotic plaques was investigated in the right and left common carotid arteries, internal and external carotid arteries (including carotid bulbs). The reproducibility of the plaque measurement was evaluated in a sample of randomly selected subjects submitted to a second ultrasound scan. The percentage of agreement and the kappa coefficient for the assessment of atherosclerotic plaques was 0.88 and 0.64 (p<0.001) respectively.

### Biological Analyses

Plasma total cholesterol and triglycerides were measured by enzymatic methods (Boehringer Mannheim, Germany). High-density lipoprotein (HDL) cholesterol was measured in the supernatant after sodium phosphotungstate/magnesium chloride precipitation (Boehringer Mannheim). Low-density lipoprotein (LDL) cholesterol was determined by the Friedewald formula. C reactive protein was measured using an immuno-enzymatic method (IBL, GmbH, Hamburg, Germany).

### DNA Amplification

Genomic DNA was previously isolated from peripheral blood cells (CHU Lille France). Total DNA concentration was then assessed using NanoDrop ND-1000 UV/Vis Spectrophotometer (NanoDrop Technologies, Wilmington, Del., USA). For quantification of total bacterial DNA, conserved region of the gene coding for 16S rRNA and bordering the variable V3 region was amplified by real-time quantitative polymerase chain reaction (RT-qPCR) with primers of the following nucleotide sequences: HDA1-forward, 5′-CCTACGGGAGGCAGCAG-3′ (SEQ ID NO: 1739); HDA2-reverse, 5′-ATTACCGCGGCTGCTGG-3′(SEQ ID NO: 1740). The 16S rDNA region amplified with these primers in the different bacterial species was from nucleotide 341 to 534 in Escherichia coli. RT-qPCR was performed with Stepone Plus Real-Time PCR System on Microamp Fast Optical 96-wells reaction plates (Applied Biosystems, Foster City, Calif., USA) as follows: 1 μL of plasma DNA, 1 μL of standard DNA (E. coli DNA) or 1 μl or sterile TE (10 mM Tris-HCl pH 8.0, 1 mM EDTA) as negative control, was added to PCR mix containing 300 nM of each primer, 10 μl of sterile DEPC-treated water and 12.5 μL of Power SYBR Green PCR Master Mix (Applied Biosystems, Foster City, Calif., USA) per well in 25 μL total volume. Thermal cycling conditions were as follows: 10 min at 95° C. and 40 cycles with 15 sec at 95° C., 1 min at 55° C. and 30 sec at 72° C. In order to generate comparable data for all subjects, quantification using E. coli DNA as standard was independently performed on each plate and plasma 16S rRNA-DNA concentration was expressed as pg of 16S rRNA-DNA/ng of total DNA, representing the amount of bacterial DNA (from 16S rRNA gene) normalized by the total DNA extracted from blood cells.

Lipid A disaccharide synthase (LpxB) catalyzes the condensation of 2,3-bis(3-hydroxymyristoyl)-β-D-glucosaminyl 1-phosphate and UDP-2,3-bis(3-hydroxymyristoyl)glucosamine (lipid X) to form lipid A disaccharide. Primers used for detection of LpxB DNA by RT-qPCR were designed to amplify the LpxB gene in all bacterial genomes currently registered in NBCI database, in a region corresponding from nucleotide 154 to 299 in E. coli. Briefly, out of 1097 bacteria genomes 41 hits correspond to LpxB genomes. The alignments of the latter genomes allowed the inventors to define two regions of high homology nucleotides separating one region of low homology on the LpxB gene. These two regions were used to define primers with some degree of non-homology. The inventors ensured that at least one of the 3′ end nucleotides of each primer was C or G and absolutely conserved between all genomes. This set of primers borders a region of 146 nucleotides which amplifies various DNA fragments corresponding to LpxB genes (known and unknown sequences). The nucleotide sequences of these primers named “LpxB universal” are as follows: LPXB-forward, 5′-GAAGCNTGGTANGANATGGAAG-3′ (SEQ ID NO: 1) with N6 (degenerated nucleotide in 6^{th} position from 5′)=90% C+10% T, N12=60% C+40% T and N15=60% A+40% G; LPXB-reverse, 5′-GGNGCNTCNATNCCNACNAANACATCNGG-3′ (SEQ ID NO: 10) with N3=50% C+50% G, N6=70% C+30% T, N9=90% A+10% G, N12=50% A+15%C+35% T, N15=60% A+10% C+30% G, N18=90% A+10% G, N21=45% A+10% C+45% G and N27=10% A+10% C+40% G+40% T. Before performing RT-qPCR with these primers, the inventors tested if the expected 146 by PCR product could be obtained after final point PCR from E. coli DNA with primers specific of this strain, which nucleotide sequences are as follows: E. coli LPXB-forward, 5′-GAAGCCTGGTACGAAATGGAAG-3′ (SEQ ID NO: 2); E. coli LPXB-reverse, 5′-GGCGCATCAATACCAACAAAAACATCTGG-3′ (SEQ ID NO: 1747). Final point PCR was performed with a VWR Unocycler (VWR International bvba, Leuven, Belgium) as follows: in 25 μL total volume, 14 ng of E. coli DNA, 300 nM of each primer, 200 μM of each deoxyribonucleotide triphosphate (Sigma ref. D7295), PCR buffer (10 mM Tris-HCl pH 8.3, 50 mM KCl) (Sigma ref. P2317), 1.5 mM MgCl₂ (Sigma ref. M8787) and 16 μL of sterile DEPC-treated water, were first incubated for 5 min at 94° C. and subsequently cooled to 83° C., at which point 0.35 U of Taq DNA polymerase (Sigma ref. D4545) was added. PCR was then achieved during 28 cycles (30 sec at 94° C., 30 sec at 60° C., 1 min at 72° C.) followed by a final elongation of 10 min at 72° C. Amplification product was then analyzed by electrophoresis in 6% acrylamide gel and ethidium bromide staining.

RT-qPCR of LpxB DNA with “LpxB universal” primers from human DNA samples was then performed with Stepone Plus Real-Time PCR System on Microamp Fast Optical 96-wells reaction plates (Applied Biosystems, Foster City, Calif., USA) as follows: 1 μL of plasma DNA, 1 μL of standard DNA (E. coli DNA) or 1 μL of sterile TE (10 mM Tris-HCl pH 8.0, 1 mM EDTA) as negative control, was added to PCR mix containing per well in 25 μL total volume, 600 nM of each primer, 8.5 μL of sterile DEPC-treated water and 12.5 μL of Power SYBR Green PCR Master Mix (Applied Biosystems, Foster City, Calif., USA). Thermal cycling conditions were as follows: 10 min at 95° C. and 50 cycles with 15 sec at 95° C., 1 min at 60° C. and 30 sec at 72° C. In order to generate data comparable for all the subjects, quantification using E. coli DNA as standard was independently performed on each plate. Plasma LpxB DNA concentration was first expressed in pg LpxB DNA/ng of total DNA, representing total LpxB DNA normalized by the total DNA extracted from peripheral blood cells, as an index of peripheral blood Gram-negative specific bacteria. Plasma LpxB DNA was also normalized by performing the ratio between the concentration of LpxB DNA and 16S rRNA-DNA and expressed in % of total DNA. LpxA (UDP-N-acetylglucosamine acyltransferase) catalyzes the first reaction of lipid A biosynthesis. Similarly, primers were designed and the corresponding PCR product quantified. Briefly, “LpxA universal” primers were designed to amplify the LpxA gene in all bacterial genomes, in a region from nucleotide 1 to 224 in E. coli. The nucleotide sequences of these primers were as follows: LPXA-forward, 5′-GTGATTGANAAANCCGCCTTTNTNCATCC-3′ (SEQ ID NO: 1741) with N9=30% C+70% T, N13=40% A+60% T, N22=60% A+40% G and N24=30% C+40% G+30% T; LPXA-reverse, 5′-ANATCNTGNTTNNCTTCNCC-3′ (SEQ ID NO: 1742) with N2=10% C+90% G, N6=80% C+20% T, N9=10% A+90% G, N12=45% A+45% C+10% T, N13=75% A+25% G and N18=50% A+50% G. The expected 224 by PCR product was first amplified by final point PCR from E. coli DNA with specific primers. The specific sequences were as follows: E. coli LPXA-forward, 5′-GTGATTGATAAATCCGCCTTTGTGCATCC-3′ (SEQ ID NO: 1743); E. coli LPXA-reverse, 5′-AGATCCTGGTTAACTTCGCC-3′ (SEQ ID NO: 1744). Final point PCR was performed with the same protocol used for E. coli LpxB and final product visualized after electrophoresis on acrylamide gel and ethidium bromide staining.

RT-qPCR of LpxA DNA from human DNA samples with “LpxA universal” primers was then performed with the same protocol used for LpxB DNA RT-qPCR. Plasma LpxA DNA concentration was expressed in pg LpxA DNA/ng total DNA and as a ratio between the concentration of LpxA DNA and 16S rRNA-DNA and expressed in % of total DNA.

### Statistical Analysis

Statistical analyses were performed with SAS statistical software release 8.2 (SAS Institute Inc, Cary, N.C., USA). The distribution of bacterial DNA values was highly skewed, with about 40% of values below the level of detection. Thus, the inventors analyzed bacterial DNA concentration as a dichotomous outcome according to the median. Also they defined as glucose metabolism abnormality patients with fasting glycemia>6.1 mmol/l and/or treated diabetes. A bivariate analysis between bacterial DNA concentration and clinical and biological variables was made using Spearman rank correlation. The relationships between the number of carotid plaques and LpxB concentration was assessed using a general linear model. The relationships between glucose metabolism abnormality and LpxB were assessed using multivariate logistic regression. P<0.05 was considered as statistically significant.

### Results

311 subjects were analyzed. Weight, waist, fasting glycemia, prevalence of treated diabetes and glucose metabolism abnormality, pulse wave velocity and the number of carotid plaques were found significantly higher in patients with LpxB above the median value (Table 1).

TABLE 1
Characteristics of study population

| | LpxB/total DNA ≦ 0.029 | | LPXb/total DNA > 0.029 | | |
|---|---|---|---|---|---|
| | N = 155 | | N = 156 | | p |
| Age | 53.14 ± 6.99 | | 54.17 ± 7.54 | | 0.20 |
| Systolic BP (mm Hg) | 134.11 ± 15.06 | | 137.22 ± 16.31 | | 0.08 |
| Diastolic BP (mm Hg) | 82.74 ± 9.06 | | 83.56 ± 9.13 | | 0.62 |
| Pulse pressure (mm Hg) | 51.36 ± 11.49 | | 53.66 ± 12.50 | | 0.09 |
| Treated hypertension | 16 | (10.32) | 25 | (16.03) | 0.13 |
| Current smokers | 32 | (20.65) | 34 | (21.79) | 0.80 |

Arterial parameters

| | | | |
|---|---|---|---|
| Pulse wave velocity (m/s) | 9.21 ± 1.73 | 9.66 ± 1.97 | 0.03 |
| Number of carotid plaques | .60 ± .88 | 1.00 ± 01.25 | 0.001 |

Metabolic and inflammatory parameters

| Weight (Kg) | 75.79 ± 10.69 | | 79.48 ± 13.30 | | 0.007 |
|---|---|---|---|---|---|
| Waist (cm) | 92.26 ± 9.12 | | 95.02 ± 11.37 | | 0.01 |
| Body mass index (Kg/m²) | 25.66 ± 3.20 | | 26.89 ± 4.11 | | 0.003 |
| Fasting glycemia (mmol/l) | 5.69 ± 0.58 | | 6.16 ± 1.55 | | 0.0005 |
| Total cholesterol (mmol/l) | 5.95 ± 0.90 | | 6.04 ± 1.10 | | 0.42 |
| Triglycerides (mmol/l)) | 1.39 ± 1.10 | | 1.50 ± 0.95 | | 0.07* |
| HDL cholesterol (mmol/l) | 1.52 ± 0.47 | | 1.54 ± 0.47 | | 0.75 |
| Treated diabetes | 1 | (0.65) | 11 | (7.05) | 0.003 |
| Glucose metabolism | 24 | (15.48) | 59 | (37.82) | <0.0001 |
| abnormality | | | | | |
| Treated dyslipidemia | 12 | (7.74) | 19 | (12.18) | 0.19 |

| | | | |
|---|---|---|---|
| C reactive protein (mg/l)* | 1.81 ± 3.16 | 1.85 ± 2.62 | 0.67 |
| Interleukin 6 ^{$} | 3.30 ± 2.72 | 4.83 ± 3.99 | 0.0002 |
| Leukocyte count | 6.59 ± 1.83 | 6.29 ± 1.85 | 0.14 |

Glucose metabolism abnormality was defined by fasting glycemia >6.1 mmol/l and/or treated diabetes.
*log transformed
^{$} the analysis was performed in 290 subjects only.

Using Spearman rank test, the inventors found that plasma LpxB DNA concentration correlated with weight, waist, fasting glycemia, prevalence of treated diabetes and glucose metabolism abnormality and number of carotid plaques whereas bacterial DNA correlates with metabolic parameters but not with carotid plaques (Table 2).

TABLE 2
Spearman rank test

| | LpxB | | 16S rDNA | |
|---|---|---|---|---|
| | R | P | R | P |
| Age | 0.06 | 0.23 | 0.02 | 0.65 |

Blood pressure parameters

| | | | | |
|---|---|---|---|---|
| Systolic BP | 0.13 | 0.01 | 0.08 | 0.14 |
| Diastolic BP | 0.05 | 0.35 | 0.05 | 0.29 |
| Treated hypertension | 0.07 | 0.18 | 0.02 | 0.65 |

Arterial parameters

| | | | | |
|---|---|---|---|---|
| Pulse wave velocity | 0.08 | 0.13 | 0.05 | 0.29 |
| Number of carotid plaques | 0.15 | 0.005 | 0.09 | 0.08 |

Metabolic parameters and inflammatory parameters

| | | | | |
|---|---|---|---|---|
| Weight | 0.12 | 0.02 | 0.14 | 0.01 |
| Waist | 0.13 | 0.01 | 0.12 | 0.03 |
| Body mass index (kg/m²) | 0.16 | 0.002 | 0.18 | 0.001 |
| Glycaemia | 0.23 | <0.0001 | 0.34 | <0.0001 |
| Total cholesterol (mmol/l) | 0.02 | 0.70 | 0.01 | 0.73 |
| Triglycerides (mmol/l) | 0.10 | 0.05 | 0.18 | 0.0008 |
| HDL cholesterol (mmol/l) | 0.05 | 0.37 | −0.18 | 0.001 |
| Interleukin 6 | 0.28 | <0.0001 | −0.03 | 0.53 |
| Leukocyte count | −0.13 | 0.02 | 0.009 | 0.86 |
| C reactive protein (mg/l) | 0.03 | 0.59 | 0.05 | 0.34 |
| Treated diabetes | 0.16 | 0.003 | 0.18 | 0.001 |
| Glucose metabolism | 0.32 | <0.0001 | 0.41 | <0.0001 |
| abnormality* | | | | |
| Treated dyslipidemia | 0.07 | 0.16 | −0.01 | 0.79 |

Glucose metabolism abnormality was defined by fasting glycemia >6.1 mmol/l and/or treated diabetes.

In multivariate logistic regression, LpxB was independently associated with glucose metabolism abnormality after adjustment for traditional risk factors (Table 3).

TABLE 3
Determinants of the absence of glucose metabolism abnormality
with traditional risk factors and LpxB as dependent variables

| | estimate | 95% confidence intervals | | p |
|---|---|---|---|---|
| Age (per one year increase) | 1.002 | 0.96 | 1.04 | 0.91 |
| LpxB (<50 th vs >50^{th} centiles) | 3.00 | 1.68 | 5.36 | 0.0002 |
| Systolic BP (per 1 mm Hg increase) | 1.02 | 1.00 | 1.04 | 0.008 |
| Waist (per 1 cm increase) | 1.06 | 1.03 | 1.10 | 0.0002 |
| HDL (per 1 mmol/l increase) | 0.53 | 0.27 | 1.06 | 0.07 |
| C reactive protein (per 1 mg/1 increase)* | 0.84 | 0.62 | 1.13 | 0.25 |

*log transformed
Glucose metabolism abnormality was defined by fasting glycemia >6.1 mmol/l and/or treated diabetes.

However this association was no more statistically significant when 16S rRNA-DNA was added in the model (Table 4).

TABLE 4
Determinants of the absence of glucose metabolism abnormality
with traditional risk factors and both LpxBand 16S DNA as dependent variables

| | estimate | 95% confidence intervals | | p |
|---|---|---|---|---|
| Age (per one year increase) | 0.99 | 0.94 | 1.03 | 0.75 |
| DNA (<50 th vs >50^{th} centiles) | 7.05 | 3.24 | 15.37 | <0.0001 |
| LpxB (<50 th vs >50^{th} centiles) | 1.27 | 0.64 | 2.52 | 0.49 |
| Systolic BP (per 1 mm Hg increase) | 1.02 | 1.007 | 1.044 | 0.008 |
| Waist (per 1 cm increase) | 1.09 | 1.05 | 1.13 | <0.0001 |
| HDL (per 1 mmol/l increase) | 0.83 | 0.41 | 1.70 | 0.62 |
| C reactive protein (per 1 mg/l increase) | 0.79 | 0.57 | 1.10 | 0.17 |

Conversely, 16S rRNA-DNA remained independently correlated with glucose metabolism abnormality. Finally, LpxB was independently associated with the number of carotid plaques (Table 5).

TABLE 5
Independent determinants of the number of carotid plaques ^{§}

| | F value | P value |
|---|---|---|
| Age (per one year increase) | 14.32 | 0.0002 |
| LpxB * | 4.22 | 0.04 |
| Systolic BP (per one mm Hg increase) | 34.26 | <0.0001 |
| glucose metabolism abnormality | 1.03 | 0.31 |
| interleukin 6 | 1.55 | 0.11 |
| Smoking status ^{$} | 2.85 | 0.05 |
| HDL (per 1 mmol/l increase) | 2.56 | 0.28 |

^{§} log(1 + x) transformed
* LpxB > 50^{th} = 1 and LpxB ≦ 50^{th} = 0;
^{$} current smokers = 1 and no smokers = 0;
Glucose metabolism abnormality was defined by fasting glycemia >6.1 mmol/l and/or treated diabetes.

Importantly no correlation was observed with LpxA showing the specificity of LpxB and total 16S rRNA-DNA for the correlated parameters.

Accordingly, the inventors demonstrated that 1) bacterial DNA could be detected in blood of apparently healthy men, 2) the concentration of 16S rRNA-DNA correlated with glucose metabolism abnormality, and 3) the concentration of LpxB DNA correlated with the number of atherosclerotic carotid plaques.

### Independent Correlation Between 16S DNA and Glucose Metabolism Abnormality

In the present study, the inventors found an independent correlation between LpxB and glucose metabolism abnormality. However, global bacteria burden in blood as assessed by 16S rRNA-DNA appeared as a better predictor of metabolic diseases than LpxB. These results demonstrate that bacteria distinct from those recognized by the primer used by the inventors contribute to metabolic diseases. However, with respect to the cross sectional nature of the present data, these results do not rule out the triggering role on the onset of metabolic diseases of bacteria coding for LPS as previously found in animal models. It is of note that odds ratio of having metabolic diseases was of 7 in patients in the top of distribution of 16S rRNA-DNA and this relation is independent from traditional risk factors such as waist. Furthermore, this relation is independent of inflammatory markers such as C reactive protein and interleukin 6. These data thus confirmed that 16S rRNA-DNA could be an innovative and potent marker to predict the occurrence of metabolic diseases in subjects at risk. Also, it could be speculated from these results that changes in microbiota could be a therapeutic target to prevent diabetes.

### LpxB Predicts the Ocurrence of Atherosclerotic Plaques

The inventors found an independent correlation between the concentration of LpxB DNA and plaques. They demonstrated that LpxB DNA blood concentration was connected with atherosclerosis process. Importantly, their results suggest that among microbiota, specifically, bacteria carrying the LpxB gene have a deleterious impact on atherosclerosis process. It is likely that a specific component of LPS could play a key role on atherosclerosis process. Importantly this correlation is independent from CRP, interleukin 6 and leucocyte count suggesting first that interleukin 6 and CRP are not involved in this relation. Additionally, the inventors did not find any significant association in a sub-sample of patients between plasma LPS concentration and LpxB DNA and between plasma LPS concentration and plaques. In this respect, LpxB DNA is a more reliable marker of atherosclerotic disease than endotoxin.

### EXAMPLE 2

The following example shows the role of 16S DNA blood concentration as a marker of bacterial translocation on the onset of metabolic disease in a large sample of the general population.

### Methods

### Population

D.E.S.I.R. is a longitudinal cohort study of 5,212 adults aged 30-64 years at baseline with the primary aim of describing the natural history of the metabolic syndrome (Cauchy et aL (2006) Diabetes 55:3189-3192). Subjects were recruited from 1994 to 1996 from 10 different Social Security Health Examination centres in western-central France among volunteers insured by the French national social security system (80% of the French population, any employed or retired person and their dependents which offers free periodic health examinations). As part of the recruitment design, men and women were recruited equally among five-year age groups. All subjects gave written informed consent, and the study protocol was approved by the Comité Consultatif de Protection des Personnes pour la Recherche Biomédicale of Bicêtre hospital (Paris, France). Participants were clinically and biologically evaluated at inclusion and at 3-, 6- and 9-year follow-up visits.

### Data Analysis

The inventors assessed the relation between 16S DNA blood concentration in subjects free of diabetes at inclusion and the onset of diabetes at different times of follow-up. Then, in order to test the predictive value of bacterial 16S DNA in subjects at low risk of diabetes according to established risk factors, they performed a subgroup analysis in subjects free of metabolic syndrome according to NCEP-ATP (National Cholesterol Education Program—Adult Treatment Panel) criteria at inclusion (National Institutes of Health: Third Report of the National Cholesterol Education Program Expert Panel on Detection, Evaluation and Treatment of High Blood Cholesterol in Adults (Adult Treatment Panel III). Executive Summary. Bethesda, Md., National Institutes of Health, National Heart, Lung and Blood Institute, 2001 (NIH publ. no. 01-3670)) or in subjects with waist perimeter below the median of waist perimeter according to the sex.

According to NCEP-ATP criteria, a participant has the metabolic syndrome if he or she has three or more of the following criteria:

1) Abdominal obesity: waist circumference>102 cm in men and >88 cm in women.

2) Hypertriglyceridemia: ≧1.7 mmol/l

3) Low HDL cholesterol: <1.03 mmol/l in men and <1.29 mmol/l in women

4) High blood pressure: ≧130/85 mmHg

5) High fasting glucose: ≧6.1 mmol/l

The inventors assessed the relation between 16S DNA concentration in the whole DESIR cohort with available data except patients with diabetes at baseline.

An univariate analysis was first performed to explore the relation between the onset of diabetes and 16S DNA concentration at different times of follow-up. The inventors analysed separately the early onset of diabetes (between inclusion and year 3 [0-3 years] follow-up period) and a late onset of diabetes (after year 3 until the end of follow-up ]3-9 years follow-up]). A multivariate logistic regression was then conducted with diabetes as dependent variable and 16S DNA blood concentration. The model was adjusted on age and sex and major risk factors for diabetes. Then subgroup analyses were performed.

Since the distribution of 16S DNA blood concentration was highly skewed, this quantitative variable was transformed into a dichotomous variable according to the median of the distribution.

### Results

The characteristics of the study population are shown in Table 6.

TABLE 6
Characteristics of the study population (n = 4637)

| | | | percentiles | | |
|---|---|---|---|---|---|
| | Mean | Sd | 25th | 50th | 75^{th} |
| Age (years) | 46.76 | 10.02 | 38 | 46 | 55 |
| BMI (kg/m2) | 24.63 | 3.73 | 22.05 | 24.16 | 26.70 |
| Fasting blood glucose (mmol/l) | 5.28 | 0.53 | 4.91 | 5.24 | 5.62 |
| Triglycerides (mmol/l) | 1.14 | 0.76 | 0.67 | 0.95 | 1.39 |
| Waist perimeter (cm) | | | | | |
| men | 89.34 | 9.25 | 83 | 89 | 95 |
| women | 79.96 | 10.21 | 66 | 70 | 75 |
| 16S DNA (ng/μl) | 0.131 | 0.302 | 0.031 | 0.0578 | 0.119 |

| | N | % | |
|---|---|---|---|
| Male | 2271 | 48.98 | |
| Current smokers | 1014 | 21.87 | |
| Hypertension (treated patients or | 1674 | 36.10 | |
| blood pressure >=140/90 mm Hg) | | | |
| Moderate or intense physical activity | 955 | 20.60 | |
| Metabolic syndrome (NCEP criteria) | 369 | 7.96 | |
| Familial history of diabetes | 903 | 19.47 | |
| | | | Missing data |
| Incident diabetes | | | |
| Through the whole follow-up period | 179 | 5.4 | 1325 |
| Early onset of diabetes: between ]0-3] | | | 625 |
| years follow-up period | | | |
| Late onset of diabetes: between ]3-9] | 102 | 3.15 | 1402 |
| years follow-up period | | | |

In univariate analysis (Table 7) the incidence of late onset of diabetes was higher in subjects in the top of the distribution of bacterial 16S DNA (median).

TABLE 7
Analysis of variance between baseline 16S DNA concentration and
onset of diabetes at different time of follow-up

| | 16S DNA | | |
|---|---|---|---|
| | ≦median | >median | p |
| Incident diabetes | | | |
| Early onset of diabetes ]0-3] years | 38 (49.35) | 39 (50.65) | 0.90 |
| of follow-up | | | |
| Late onset of diabetes ]3-9] years | 41 (40.20) | 61 (59.80)* | 0.03 |
| follow-up period | | | |
| Free of diabetes during the whole | 1599 (51.04) | 1534 (48.96) | |
| follow-up period | | | |

Percentages are in brackets.

The comparisons were performed between subjects who experienced diabetes at different times and subjects free of diabetes during the whole follow-up.

This relation remained significant after adjustment for confounder (Tables 8 and 9). Then a subgroup analysis was performed in subjects at low risk for diabetes. In subjects in the bottom of the distribution of waist perimeter according to sex (≦89 cm in men and ≦72 cm in women) and in subjects free of metabolic syndrome at inclusion, 27 and 102 diabetes respectively occurred after year 3. In both these subgroups, bacterial 16S DNA remained positively and independently associated with the late onset of diabetes.

TABLE 8
Logistic regression analysis with late onset of diabetes (n = 27) as
dependent variable in patients free of diabetes until year 3 and in the bottom of the
distribution of waist perimeter (≦median according to sex).

| | | Lower confidence | Upper Confidence | |
|---|---|---|---|---|
| | Odds ratio | interval limit (95%) | interval limit (95%) | P |
| Smoker (yes versus | 4.42 | 1.92 | 10.17 | 0.0005 |
| no) | | | | |
| 16S DNA (above vs | 2.63 | 1.12 | 6.20 | 0.02 |
| below the median) | | | | |
| Fasting glucose at | 14.83 | 6.91 | 31.85 | <0.0001 |
| entry (per 1 mmol/l | | | | |
| increase) | | | | |

With respect to the number of diabetes, this model was adjusted for smoking status and fasting glucose only.

TABLE 9
Logistic regression analysis with late onset of diabetes (n = 102) as
dependent variable among patients free of diabetes until year 3 and free of
metabolic syndrome at inclusion.

| | | Lower confidence | Upper Confidence | |
|---|---|---|---|---|
| | Odds ratio | interval limit (95%) | interval limit (95%) | P |
| Age (per one year | 0.99 | 0.97 | 1.02 | 0.93 |
| increase) | | | | |
| Male vs female | 0.60 | 0.35 | 1.00 | 0.05 |
| Smoker (yes versus no) | 2.59 | 1.57 | 4.25 | 0.0002 |
| Waist (per 1 cm | 1.07 | 1.05 | 1.10 | <0.0001 |
| increase) | | | | |
| 16S DNA (above vs | 2.10 | 1.35 | 3.27 | <0.001 |
| below the median) | | | | |
| Hypertension | 0.98 | 0.61 | 1.55 | 0.93 |
| Fasting glucose at | 9.26 | 5.95 | 14.39 | <0.0001 |
| entry (per 1 mmol/l | | | | |
| increase) | | | | |
| Physical activity | 1.14 | 0.65 | 2.00 | 0.63 |
| Familial history of | 1.37 | 0.83 | 2.25 | 0.20 |
| diabetes | | | | |

In conclusion, the inventors here confirmed that 16S DNA blood concentration is an independent predictive marker of the onset of diabetes. Furthermore they demonstrated that bacterial 16S DNA predicted the risk of diabetes in subjects free of central adiposity and metabolic syndrome. They also demonstrated that 16S DNA blood concentration was an independent predictive marker of the late onset of diabetes.

### EXAMPLE 3

The following example describes the way the inventors improved a method for the determination of 16S DNA in tissues by discriminating between bacterial 16S DNA and mitochondrial DNA.

The mitochondrial and the bacterial DNA have a similar origin and primers usually used in the literature cross react with both types of DNA. Therefore, PCR techniques cannot discriminate between both DNA origin since they amplify altogether. The solution proposed by the inventors is based on the fact that bacterial DNA and mitochondrial DNA do not have the same GC content and hence the temperatures required to dissociate the double strand DNA sequences are different. The inventors have standardized the PCR procedure and the dissociation curves and identified the best fit formulation which allows discrimination between both DNA origins.

### Methods

### Set Up of the 16S DNA Standard Curve

DNA from a fresh culture of Escherichia coli BL21 (prokaryotic 16S DNA) or from axenic (germ free, no bacterial DNA) mouse liver (eukaryotic mitochondrial DNA) were extracted using TriPure Isolation Reagent (Roche Applied Science, Germany) according to manufacturer's instructions. Standard DNA samples were obtained by mixing increasing quantities of prokaryotic DNA (ranging from 0.001 to 1000 ng) with a fixed quantity of eukaryotic DNA (100 ng). A PCR reaction was then performed as follows.

### PCR Conditions

Amplification and detection of DNA by real time PCR was performed using Stepone Plus Real-Time PCR System and Microamp Fast Optical 96-wells reaction plates (Applied Biosystems, California, USA).

Primers:
(SEQ ID NO: 1748)
HDA1-F, 5′-ACTCCTACGGGAGGCAGCAGT-3′;
(SEQ ID NO: 1749)
HDA2-R, 5′-GTATTACCGCGGCTGCTGGCAC-3′.

Duplicate samples were used for the determination of DNA by real time PCR. The PCR reaction was performed in a total volume of 25 μL using the Power SYBR® Green PCR master mix (Applied Biosystems, California, USA) and 300 nM of each of the universal forward and reverse primers HDA1 and HDA2. Thermal cycling conditions for amplification of DNA were 95° C. for 10 min followed by 42 cycles each comprising 95° C. for 15 s, 55° C. for 1 min and 72° C. for 30 s. The amplification stage was followed by a melting curve stage (dissociation curve) according to the manufacturer's instructions (from 55° C. to 90° C.) to determine the specificity of the amplification.

### Results

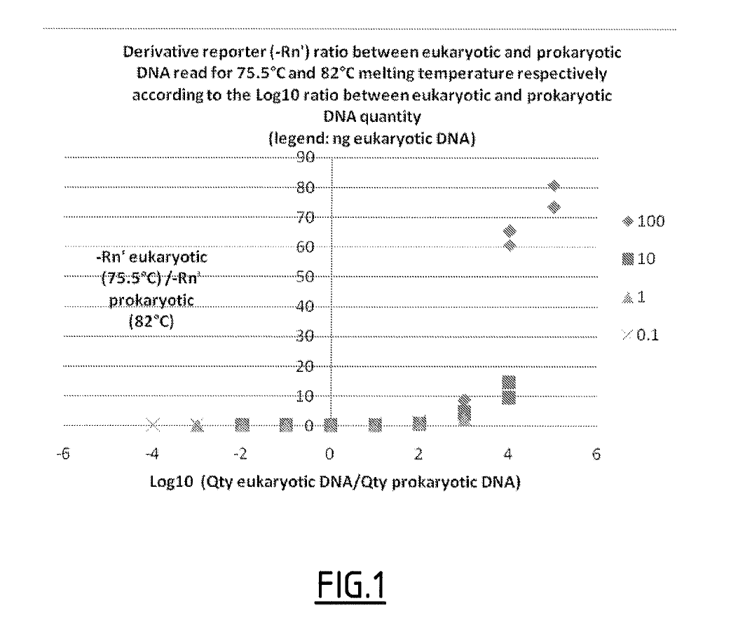

Four dose response curves were determined with fixed quantities of eukaryotic DNA (0.1; 1; 10 or 100 ng) and increasing quantities of prokaryotic DNA from 0.001 to 1000 ng for each fixed eukaryotic DNA quantity (FIG. 1).

Melting curve data allowed discriminating amplification of either prokaryotic or eukaryotic DNA as follows. The 75.5° C. temperature and 82° C. temperature corresponded to the melting (dissociation) temperature of the eukaryotic mitochondrial DNA and the 16S DNA respectively. The negative first-derivative of the normalized fluorescence (−Rn′) generated by the reporter (SYBR Green) during PCR amplification was used.

The ratio between derivative reporter value read at 75.5° C. and 82° C. was calculated and plotted against the Log 10 of ng eukaryotic DNA/ng prokaryotic DNA to establish a dose response curve.

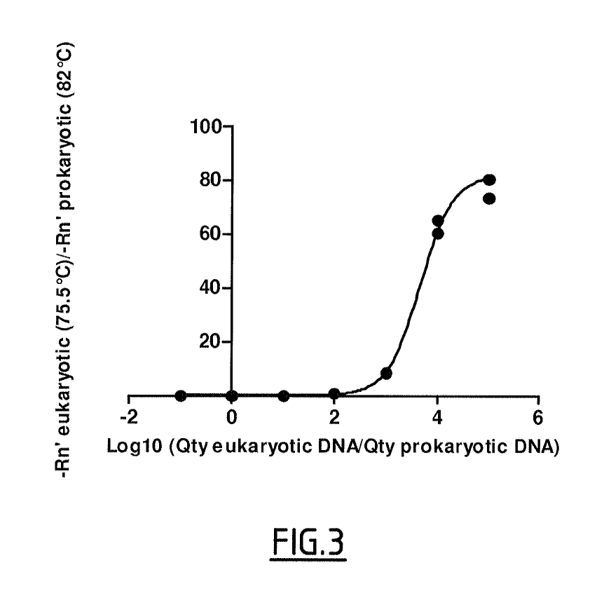

The calculation of the best fit formulation (FIG. 3) allowed the identification of the concentration of the bacterial DNA out of the concentration of the mitochondrial DNA.

The following equation was established to calculate the ratio between eukaryotic and prokaryotic DNA:

Y=bottom+(top−bottom)/[1+10̂((Log EC50−X)*HillSlope)]

with

X=Log 10(eukaryotic DNA/prokaryotic DNA),

Y=−Rn′ read at 77.5° C./−Rn′ read at 82° C.,

bottom=0.1903,

top=81.94,

Log EC50=3.673,

HillSlope=1.39, and

EC50=4709.

Therefore, in an unknown sample the plotting of the ratio between both DNA species allowed the quantification of each specie.

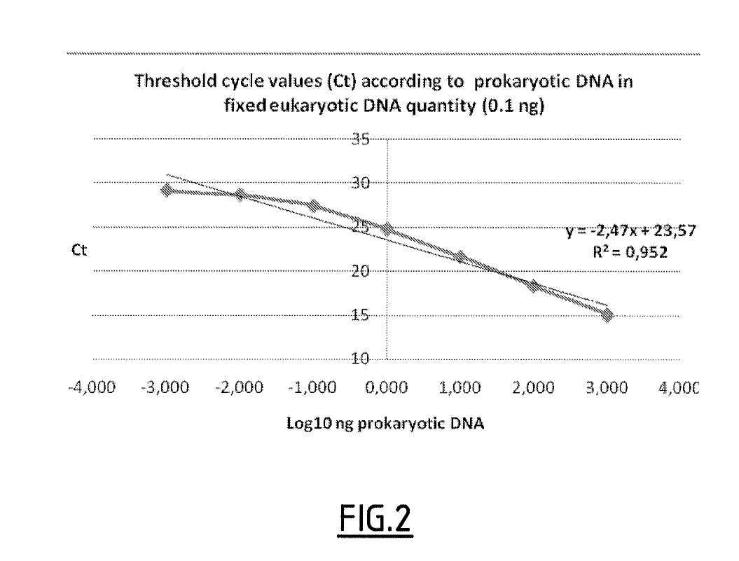

For comparison, a dose response curve of threshold cycle values (Ct) according to Log 10 ng prokaryotic DNA was established by incubating increasing quantities of prokaryotic DNA with a fixed quantity of eukaryotic DNA (0.1 ng) (FIG. 2).

Importantly, the method using the melting temperatures was ten times more sensitive that the Ct technique since it allowed the quantification of 0.001 ng of DNA.

In conclusion, the method designed by the inventors is summarized as follows:
PCR using the primers as cited above to perform the dose response of bacterial DNA versus eukaryotic DNA
Definition of the melting temperature and dose response curve
Definition of the best fit equation
PCR using primers as cited above from the unknown samples
Definition of the melting temperature
Plotting of the melting temperature on the dose response curve
Determination of the 16S rRNA DNA concentration.

### EXAMPLE 4

The following example describes primers specific of 16S DNA usable in the methods of the invention.

The inventors designed primers which did not cross react with the mitochondrial DNA and which were specific for 16S DNA to quantify the amount of bacterial DNA present in a tissue and which would define a prediabetic state.

### Method

The inventors used the following primers: EUBAC-F, 5′-TCCTACGGGAGGCAGCAGT-3′ (SEQ ID NO: 1750); EUBAC-R, 5′-GGACTACCAGGGTATCTAATCCTGTT-3′ (SEQ ID NO: 1751).

Amplification and detection of 16S DNA by real time PCR was performed with

Stepone Plus Real-Time PCR System on Microamp Fast Optical 96-wells reaction plates (Applied Biosystems, California, USA) as follows: 2 μL of DNA were added to PCR mix comprising per well (25 μL total volume), 300 nM of each primer, 9 μL of sterile DEPC-treated water and 12.5 μL of Power SYBR® Green PCR Master Mix (Applied Biosystems, California, USA).

Thermal cycling conditions were as follows: 95° C. for 10 min and 42 cycles each involving 95° C. for 15 s, 60° C. for 1 min and 72° C. for 30 s.

### Results

These primers allowed the amplification of a DNA product which was unique and coded for a fraction of the 16S DNA gene.

In conclusion, these primers can be used directly to quantify the bacterial DNA by PCR and establish the prediagnosis.

1. An in vitro method for predicting and/or diagnosing a cardiovascular and/or metabolic disease in a subject, which method comprises determining the concentration of bacterial DNA in a biological sample of said subject.

2. The method according to claim 1, wherein bacterial DNA is 16S DNA and/or LpxB DNA.

3. The method according to claim 1, wherein said method is for predicting and/or diagnosing a cardiovascular disease in a subject and comprises determining the LpxB DNA concentration in the biological sample of said subject.

4. The method according to any one of claim 1, wherein said cardiovascular disease is selected from the group consisting in coronary artery disease, hypertension, atherosclerosis, vascular aneurysm, vascular calcification, vascular dementia and heart failure.

5. The method according to claim 1, wherein said method is for predicting and/or diagnosing a metabolic disease in a subject and comprises determining the bacterial 16S DNA concentration in the biological sample of said subject.

6. The method according to claim 5, which further comprises determining the LpxB DNA concentration in the biological sample of said subject.

7. The method according to claim 1, wherein said metabolic disease is a carbohydrate metabolism disorder.

8. An isolated nucleic acid which comprises a sequence selected from the group consisting of the sequence 5′-GAAGCNTGGTANGANATGGAAG-3′ (SEQ ID NO: 1), wherein N in 6^{th} position from 5′ is 90% C or 10% T, N in 12^{th} position from 5′ is 60% C or 40% T and N in 15^{th} position from 5′ is 60% A or 40% G, and the sequence 5′-GGNGCNTCNATNCCNACNAANACATCNGG-3′ (SEQ ID NO: 10), wherein N in 3^{rd} position from 5′ is 50% C or 50% G, N in 6^{th} position from 5′ is 70% C or 30% T, N in 9^{th} position from 5′ is 90% A or 10% G, N in 12^{th} position from 5′ is 50% A, 15% C or 35% T, N in 15^{th} position from 5′ is 60% A, 10% C or 30% G, N in 18^{th} position from 5′ is 90% A or 10% G, N in 21^{st} position from 5′ is 45% A, 10% C or 45% G and N in 27^{th} position from 5′ is 10% A, 10% C, 40% G or 40% T.

9. A pair of primers comprising a first and a second primer, wherein the first primer comprises a nucleic acid which consists of the sequence 5′-GAAGCNTGGTANGANATGGAAG-3′ (SEQ ID NO: 1), wherein N in 6^{th} position from 5′ is 90% C or 10% T, N in 12^{th} position from 5′ is 60% C or 40% T and N in 15^{th} position from 5′ is 60% A or 40% G; and the second primer comprises a nucleic acid which consists of the sequence 5′-GGNGCNTCNATNCCNACNAANACATCNGG-3′ (SEQ ID NO: 10), wherein N in 3^{rd} position from 5′ is 50% C or 50% G, N in 6^{th} position from 5′ is 70% C or 30% T, N in 9^{th} position from 5′ is 90% A or 10% G, N in 12^{th} position from 5′ is 50% A, 15% C or 35% T, N in 15^{th} position from 5′ is 60% A, 10% C or 30% G, N in 18^{th} position from 5′ is 90% A or 10% G, N in 21^{st} position from 5′ is 45% A, 10% C or 45% G and N in 27^{th} position from 5′ is 10% A, 10% C, 40% G or 40% T.

10. (canceled)

11. (canceled)

12. A kit for predicting and/or diagnosing a metabolic and/or cardiovascular disease in a subject, wherein said kit comprises at least one nucleic acid that hybridizes specifically under high stringency conditions to LpxB DNA.

13. The kit according to claim 12, wherein said kit further comprises at least one nucleic acid that hybridizes specifically under high stringency conditions to bacterial 16S DNA.

14. A method for screening probiotics, prebiotics or chemical or biological compounds suitable for preventing and/or treating metabolic and/or cardiovascular diseases, comprising determining bacterial 16S DNA concentration and/or LpxB DNA concentration in a biological sample of a subject which has been treated with the candidate probiotic, prebiotic or chemical or biological compound and comparing said concentrations with those of a control subject which has not been treated.

15. An in vitro method for identifying predictive markers of a cardiovascular and/or metabolic disease, comprising
(i) detecting at least one sequence of bacterial DNA in a biological sample of an apparently healthy subject,
(ii) analyzing parameters that are indicative of a cardiovascular disease in said subject; and
(iii) identifying as predictive markers the sequences detected in the biological sample of said subject which are positively associated with the parameters that are indicative of a cardiovascular metabolic disease.


SEQUENCE LISTING (1751 sequences)

SEQ ID NO: 1 - DNA, 22 bp, Artificial
  primer LPXB forward
gaagcytggtaygaratggaag

SEQ ID NO: 2 - DNA, 22 bp, Artificial
  primer LPXB forward 1
gaagcctggtacgaaatggaag

SEQ ID NO: 3 - DNA, 22 bp, Artificial
  primer LPXB forward 2
gaagcttggtacgaaatggaag

SEQ ID NO: 4 - DNA, 22 bp, Artificial
  prmer LPXB forward 3
gaagcctggtatgaaatggaag

SEQ ID NO: 5 - DNA, 22 bp, Artificial
  primer LPXB forward 4
gaagcttggtatgaaatggaag

SEQ ID NO: 6 - DNA, 22 bp, Artificial
  primer LPXB forward 5
gaagcctggtacgagatggaag

SEQ ID NO: 7 - DNA, 22 bp, Artificial
  primer LPXB forward 6
gaagcttggtacgagatggaag

SEQ ID NO: 8 - DNA, 22 bp, Artificial
  primer LPXB forward 7
gaagcttggtacgagatggaag

SEQ ID NO: 9 - DNA, 22 bp, Artificial
  primer LPXB forward 8
gaagcttggtatgagatggaag

SEQ ID NO: 10 - DNA, 29 bp, Artificial
  primer LPXB reverse
ggsgcytcrathccvacraavacatcngg

SEQ ID NO: 11 - DNA, 29 bp, Artificial
  primer LPXB reverse 1
ggcgcctcaataccaacaaaaacatcagg

SEQ ID NO: 12 - DNA, 29 bp, Artificial
  primer LPXB reverse 2
ggcgcctcaataccaacaaaaacatccgg

SEQ ID NO: 13 - DNA, 29 bp, Artificial
  primer LPXB reverse 3
ggcgcctcaataccaacaaaaacatcggg

SEQ ID NO: 14 - DNA, 29 bp, Artificial
  primer LPXB reverse 4
ggcgcctcaataccaacaaaaacatctgg

SEQ ID NO: 15 - DNA, 29 bp, Artificial
  primer LPXB reverse 5
ggcgcctcaataccaacaaacacatcagg

SEQ ID NO: 16 - DNA, 29 bp, Artificial
  primer LPXB reverse 6
ggcgcctcaataccaacaaacacatccgg

SEQ ID NO: 17 - DNA, 29 bp, Artificial
  primer LPXB reverse 7
ggcgcctcaataccaacaaacacatctgg

SEQ ID NO: 18 - DNA, 29 bp, Artificial
  primer LPXB reverse 8
ggcgcctcaataccaacaaacacatcggg

SEQ ID NO: 19 - DNA, 29 bp, Artificial
  primer LPXB reverse 9
ggcgcctcaataccaacaaagacatcagg

SEQ ID NO: 20 - DNA, 29 bp, Artificial
  primer LPXB reverse 10
ggcgcctcaataccaacaaagacatccgg

SEQ ID NO: 21 - DNA, 29 bp, Artificial
  primer LPXB reverse 11
ggcgcctcaataccaacaaagacatcggg

SEQ ID NO: 22 - DNA, 29 bp, Artificial
  primer LPXB reverse 12
ggcgcctcaataccaacaaagacatctgg

SEQ ID NO: 23 - DNA, 29 bp, Artificial
  primer LPXB reverse 13
ggcgcctcaataccaacgaaaacatcagg

SEQ ID NO: 24 - DNA, 29 bp, Artificial
  primer LPXB reverse 14
ggcgcctcaataccaacgaaaacatccgg

SEQ ID NO: 25 - DNA, 29 bp, Artificial
  primer LPXB reverse 15
ggcgcctcaataccaacgaaaacatcggg

SEQ ID NO: 26 - DNA, 29 bp, Artificial
  primer LPXB reverse 16
ggcgcctcaataccaacgaaaacatctgg

SEQ ID NO: 27 - DNA, 29 bp, Artificial
  primer LPXB reverse 17
ggcgcctcaataccaacgaacacatcagg

SEQ ID NO: 28 - DNA, 29 bp, Artificial
  primer LPXB reverse 18
ggcgcctcaataccaacgaacacatccgg

SEQ ID NO: 29 - DNA, 29 bp, Artificial
  primer LPXB reverse 19
ggcgcctcaataccaacgaacacatctgg

SEQ ID NO: 30 - DNA, 29 bp, Artificial
  primer LPXB reverse 20
ggcgcctcaataccaacgaacacatcggg

SEQ ID NO: 31 - DNA, 29 bp, Artificial
  primer LPXB reverse 21
ggcgcctcaataccaacgaagacatcagg

SEQ ID NO: 32 - DNA, 29 bp, Artificial
  primer LPXB reverse 22
ggcgcctcaataccaacgaagacatccgg

SEQ ID NO: 33 - DNA, 29 bp, Artificial
  primer LPXB reverse 23
ggcgcctcaataccaacgaagacatcggg

SEQ ID NO: 34 - DNA, 29 bp, Artificial
  primer LPXB reverse 24
ggcgcctcaataccaacgaagacatctgg

SEQ ID NO: 35 - DNA, 29 bp, Artificial
  primer LPXB reverse 25
ggcgcctcaatacccacaaaaacatcagg

SEQ ID NO: 36 - DNA, 29 bp, Artificial
  primer LPXB reverse 26
ggcgcctcaatacccacaaaaacatccgg

SEQ ID NO: 37 - DNA, 29 bp, Artificial
  primer LPXB reverse 27
ggcgcctcaatacccacaaaaacatcggg

SEQ ID NO: 38 - DNA, 29 bp, Artificial
  primer LPXB reverse 28
ggcgcctcaatacccacaaaaacatctgg

SEQ ID NO: 39 - DNA, 29 bp, Artificial
  primer LPXB reverse 29
ggcgcctcaatacccacaaacacatcagg

SEQ ID NO: 40 - DNA, 29 bp, Artificial
  primer LPXB reverse 30
ggcgcctcaatacccacaaacacatccgg

SEQ ID NO: 41 - DNA, 29 bp, Artificial
  primer LPXB reverse 31
ggcgcctcaatacccacaaacacatctgg

SEQ ID NO: 42 - DNA, 29 bp, Artificial
  primer LPXB reverse 32
ggcgcctcaatacccacaaacacatcggg

SEQ ID NO: 43 - DNA, 29 bp, Artificial
  primer LPXB reverse 33
ggcgcctcaatacccacaaagacatcagg

SEQ ID NO: 44 - DNA, 29 bp, Artificial
  primer LPXB reverse 34
ggcgcctcaatacccacaaagacatccgg

SEQ ID NO: 45 - DNA, 29 bp, Artificial
  primer LPXB reverse 35
ggcgcctcaatacccacaaagacatcggg

SEQ ID NO: 46 - DNA, 29 bp, Artificial
  primer LPXB reverse 36
ggcgcctcaatacccacaaagacatctgg

SEQ ID NO: 47 - DNA, 29 bp, Artificial
  primer LPXB reverse 37
ggcgcctcaatacccacgaaaacatcagg

SEQ ID NO: 48 - DNA, 29 bp, Artificial
  primer LPXB reverse 38
ggcgcctcaatacccacgaaaacatccgg

SEQ ID NO: 49 - DNA, 29 bp, Artificial
  primer LPXB reverse 39
ggcgcctcaatacccacgaaaacatcggg

SEQ ID NO: 50 - DNA, 29 bp, Artificial
  primer LPXB reverse 40
ggcgcctcaatacccacgaaaacatctgg

SEQ ID NO: 51 - DNA, 29 bp, Artificial
  primer LPXB reverse 41
ggcgcctcaatacccacgaacacatcagg

SEQ ID NO: 52 - DNA, 29 bp, Artificial
  primer LPXB reverse 42
ggcgcctcaatacccacgaacacatccgg

SEQ ID NO: 53 - DNA, 29 bp, Artificial
  primer LPXB reverse 43
ggcgcctcaatacccacgaacacatctgg

SEQ ID NO: 54 - DNA, 29 bp, Artificial
  primer LPXB reverse 44
ggcgcctcaatacccacgaacacatcggg

SEQ ID NO: 55 - DNA, 29 bp, Artificial
  primer LPXB reverse 45
ggcgcctcaatacccacgaagacatcagg

SEQ ID NO: 56 - DNA, 29 bp, Artificial
  primer LPXB reverse 46
ggcgcctcaatacccacgaagacatccgg

SEQ ID NO: 57 - DNA, 29 bp, Artificial
  primer LPXB reverse 47
ggcgcctcaatacccacgaagacatcggg

SEQ ID NO: 58 - DNA, 29 bp, Artificial
  primer LPXB reverse 48
ggcgcctcaatacccacgaagacatctgg

SEQ ID NO: 59 - DNA, 29 bp, Artificial
  primer LPXB reverse 49
ggcgcctcaataccgacaaaaacatcagg

SEQ ID NO: 60 - DNA, 29 bp, Artificial
  primer LPXB reverse 50
ggcgcctcaataccgacaaaaacatccgg

SEQ ID NO: 61 - DNA, 29 bp, Artificial
  primer LPXB reverse 51
ggcgcctcaataccgacaaaaacatcggg

SEQ ID NO: 62 - DNA, 29 bp, Artificial
  primer LPXB reverse 52
ggcgcctcaataccgacaaaaacatctgg

SEQ ID NO: 63 - DNA, 29 bp, Artificial
  primer LPXB reverse 53
ggcgcctcaataccgacaaacacatcagg

SEQ ID NO: 64 - DNA, 29 bp, Artificial
  primer LPXB reverse 54
ggcgcctcaataccgacaaacacatccgg

SEQ ID NO: 65 - DNA, 29 bp, Artificial
  primer LPXB reverse 55
ggcgcctcaataccgacaaacacatctgg

SEQ ID NO: 66 - DNA, 29 bp, Artificial
  primer LPXB reverse 56
ggcgcctcaataccgacaaacacatcggg

SEQ ID NO: 67 - DNA, 29 bp, Artificial
  primer LPXB reverse 57
ggcgcctcaataccgacaaagacatcagg

SEQ ID NO: 68 - DNA, 29 bp, Artificial
  primer LPXB reverse 58
ggcgcctcaataccgacaaagacatccgg

SEQ ID NO: 69 - DNA, 29 bp, Artificial
  primer LPXB reverse 59
ggcgcctcaataccgacaaagacatcggg

SEQ ID NO: 70 - DNA, 29 bp, Artificial
  primer LPXB reverse 60
ggcgcctcaataccgacaaagacatctgg

SEQ ID NO: 71 - DNA, 29 bp, Artificial
  primer LPXB reverse 61
ggcgcctcaataccgacgaaaacatcagg

SEQ ID NO: 72 - DNA, 29 bp, Artificial
  primer LPXB reverse 62
ggcgcctcaataccgacgaaaacatccgg

SEQ ID NO: 73 - DNA, 29 bp, Artificial
  primer LPXB reverse 63
ggcgcctcaataccgacgaaaacatcggg

SEQ ID NO: 74 - DNA, 29 bp, Artificial
  primer LPXB reverse 64
ggcgcctcaataccgacgaaaacatctgg

SEQ ID NO: 75 - DNA, 29 bp, Artificial
  primer LPXB reverse 65
ggcgcctcaataccgacgaacacatcagg

SEQ ID NO: 76 - DNA, 29 bp, Artificial
  primer LPXB reverse 66
ggcgcctcaataccgacgaacacatccgg

SEQ ID NO: 77 - DNA, 29 bp, Artificial
  primer LPXB reverse 67
ggcgcctcaataccgacgaacacatctgg

SEQ ID NO: 78 - DNA, 29 bp, Artificial
  primer LPXB reverse 68
ggcgcctcaataccgacgaacacatcggg

SEQ ID NO: 79 - DNA, 29 bp, Artificial
  primer LPXB reverse 69
ggcgcctcaataccgacgaagacatcagg

SEQ ID NO: 80 - DNA, 29 bp, Artificial
  primer LPXB reverse 70
ggcgcctcaataccgacgaagacatccgg

SEQ ID NO: 81 - DNA, 29 bp, Artificial
  primer LPXB reverse 71
ggcgcctcaataccgacgaagacatcggg

SEQ ID NO: 82 - DNA, 29 bp, Artificial
  primer LPXB reverse 72
ggcgcctcaataccgacgaagacatctgg

SEQ ID NO: 83 - DNA, 29 bp, Artificial
  primer LPXB reverse 73
ggcgcctcaatcccaacaaaaacatcagg

SEQ ID NO: 84 - DNA, 29 bp, Artificial
  primer LPXB reverse 74
ggcgcctcaatcccaacaaaaacatccgg

SEQ ID NO: 85 - DNA, 29 bp, Artificial
  primer LPXB reverse 75
ggcgcctcaatcccaacaaaaacatcggg

SEQ ID NO: 86 - DNA, 29 bp, Artificial
  primer LPXB reverse 76
ggcgcctcaatcccaacaaaaacatctgg

SEQ ID NO: 87 - DNA, 29 bp, Artificial
  primer LPXB reverse 77
ggcgcctcaatcccaacaaacacatcagg

SEQ ID NO: 88 - DNA, 29 bp, Artificial
  primer LPXB reverse 78
ggcgcctcaatcccaacaaacacatccgg

SEQ ID NO: 89 - DNA, 29 bp, Artificial
  primer LPXB reverse 79
ggcgcctcaatcccaacaaacacatctgg

SEQ ID NO: 90 - DNA, 29 bp, Artificial
  primer LPXB reverse 80
ggcgcctcaatcccaacaaacacatcggg

SEQ ID NO: 91 - DNA, 29 bp, Artificial
  primer LPXB reverse 81
ggcgcctcaatcccaacaaagacatcagg

SEQ ID NO: 92 - DNA, 29 bp, Artificial
  primer LPXB reverse 82
ggcgcctcaatcccaacaaagacatccgg

SEQ ID NO: 93 - DNA, 29 bp, Artificial
  primer LPXB reverse 83
ggcgcctcaatcccaacaaagacatcggg

SEQ ID NO: 94 - DNA, 29 bp, Artificial
  primer LPXB reverse 84
ggcgcctcaatcccaacaaagacatctgg

SEQ ID NO: 95 - DNA, 29 bp, Artificial
  primer LPXB reverse 85
ggcgcctcaatcccaacgaaaacatcagg

SEQ ID NO: 96 - DNA, 29 bp, Artificial
  primer LPXB reverse 86
ggcgcctcaatcccaacgaaaacatccgg

SEQ ID NO: 97 - DNA, 29 bp, Artificial
  primer LPXB reverse 87
ggcgcctcaatcccaacgaaaacatcggg

SEQ ID NO: 98 - DNA, 29 bp, Artificial
  primer LPXB reverse 88
ggcgcctcaatcccaacgaaaacatctgg

SEQ ID NO: 99 - DNA, 29 bp, Artificial
  primer LPXB reverse 89
ggcgcctcaatcccaacgaacacatcagg

SEQ ID NO: 100 - DNA, 29 bp, Artificial
  primer LPXB reverse 90
ggcgcctcaatcccaacgaacacatccgg

SEQ ID NO: 101 - DNA, 29 bp, Artificial
  primer LPXB reverse 91
ggcgcctcaatcccaacgaacacatctgg

SEQ ID NO: 102 - DNA, 29 bp, Artificial
  primer LPXB reverse 92
ggcgcctcaatcccaacgaacacatcggg

SEQ ID NO: 103 - DNA, 29 bp, Artificial
  primer LPXB reverse 93
ggcgcctcaatcccaacgaagacatcagg

SEQ ID NO: 104 - DNA, 29 bp, Artificial
  primer LPXB reverse 94
ggcgcctcaatcccaacgaagacatccgg

SEQ ID NO: 105 - DNA, 29 bp, Artificial
  primer LPXB reverse 95
ggcgcctcaatcccaacgaagacatcggg

SEQ ID NO: 106 - DNA, 29 bp, Artificial
  primer LPXB reverse 96
ggcgcctcaatcccaacgaagacatctgg

SEQ ID NO: 107 - DNA, 29 bp, Artificial
  primer LPXB reverse 97
ggcgcctcaatccccacaaaaacatcagg

SEQ ID NO: 108 - DNA, 29 bp, Artificial
  primer LPXB reverse 98
ggcgcctcaatccccacaaaaacatccgg

SEQ ID NO: 109 - DNA, 29 bp, Artificial
  primer LPXB reverse 99
ggcgcctcaatccccacaaaaacatcggg

SEQ ID NO: 110 - DNA, 29 bp, Artificial
  primer LPXB reverse 100
ggcgcctcaatccccacaaaaacatctgg

SEQ ID NO: 111 - DNA, 29 bp, Artificial
  primer LPXB reverse 101
ggcgcctcaatccccacaaacacatcagg

SEQ ID NO: 112 - DNA, 29 bp, Artificial
  primer LPXB reverse 102
ggcgcctcaatccccacaaacacatccgg

SEQ ID NO: 113 - DNA, 29 bp, Artificial
  primer LPXB reverse 103
ggcgcctcaatccccacaaacacatctgg

SEQ ID NO: 114 - DNA, 29 bp, Artificial
  primer LPXB reverse 104
ggcgcctcaatccccacaaacacatcggg

SEQ ID NO: 115 - DNA, 29 bp, Artificial
  primer LPXB reverse 105
ggcgcctcaatccccacaaagacatcagg

SEQ ID NO: 116 - DNA, 29 bp, Artificial
  primer LPXB reverse 106
ggcgcctcaatccccacaaagacatccgg

SEQ ID NO: 117 - DNA, 29 bp, Artificial
  primer LPXB reverse 107
ggcgcctcaatccccacaaagacatcggg

SEQ ID NO: 118 - DNA, 29 bp, Artificial
  primer LPXB reverse 108
ggcgcctcaatccccacaaagacatctgg

SEQ ID NO: 119 - DNA, 29 bp, Artificial
  primer LPXB reverse 109
ggcgcctcaatccccacgaaaacatcagg

SEQ ID NO: 120 - DNA, 29 bp, Artificial
  primer LPXB reverse 110
ggcgcctcaatccccacgaaaacatccgg

SEQ ID NO: 121 - DNA, 29 bp, Artificial
  primer LPXB reverse 111
ggcgcctcaatccccacgaaaacatcggg

SEQ ID NO: 122 - DNA, 29 bp, Artificial
  primer LPXB reverse 112
ggcgcctcaatccccacgaaaacatctgg

SEQ ID NO: 123 - DNA, 29 bp, Artificial
  primer LPXB reverse 113
ggcgcctcaatccccacgaacacatcagg

SEQ ID NO: 124 - DNA, 29 bp, Artificial
  primer LPXB reverse 114
ggcgcctcaatccccacgaacacatccgg

SEQ ID NO: 125 - DNA, 29 bp, Artificial
  primer LPXB reverse 115
ggcgcctcaatccccacgaacacatctgg

SEQ ID NO: 126 - DNA, 29 bp, Artificial
  primer LPXB reverse 116
ggcgcctcaatccccacgaacacatcggg

SEQ ID NO: 127 - DNA, 29 bp, Artificial
  primer LPXB reverse 117
ggcgcctcaatccccacgaagacatcagg

SEQ ID NO: 128 - DNA, 29 bp, Artificial
  primer LPXB reverse 118
ggcgcctcaatccccacgaagacatccgg

SEQ ID NO: 129 - DNA, 29 bp, Artificial
  primer LPXB reverse 119
ggcgcctcaatccccacgaagacatcggg

SEQ ID NO: 130 - DNA, 29 bp, Artificial
  primer LPXB reverse 120
ggcgcctcaatccccacgaagacatctgg

SEQ ID NO: 131 - DNA, 29 bp, Artificial
  primer LPXB reverse 121
ggcgcctcaatcccgacaaaaacatcagg

SEQ ID NO: 132 - DNA, 29 bp, Artificial
  primer LPXB reverse 122
ggcgcctcaatcccgacaaaaacatccgg

SEQ ID NO: 133 - DNA, 29 bp, Artificial
  primer LPXB reverse 123
ggcgcctcaatcccgacaaaaacatcggg

SEQ ID NO: 134 - DNA, 29 bp, Artificial
  primer LPXB reverse 124
ggcgcctcaatcccgacaaaaacatctgg

SEQ ID NO: 135 - DNA, 29 bp, Artificial
  primer LPXB reverse 125
ggcgcctcaatcccgacaaacacatcagg

SEQ ID NO: 136 - DNA, 29 bp, Artificial
  primer LPXB reverse 126
ggcgcctcaatcccgacaaacacatccgg

SEQ ID NO: 137 - DNA, 29 bp, Artificial
  primer LPXB reverse 127
ggcgcctcaatcccgacaaacacatctgg

SEQ ID NO: 138 - DNA, 29 bp, Artificial
  primer LPXB reverse 128
ggcgcctcaatcccgacaaacacatcggg

SEQ ID NO: 139 - DNA, 29 bp, Artificial
  primer LPXB reverse 129
ggcgcctcaatcccgacaaagacatcagg

SEQ ID NO: 140 - DNA, 29 bp, Artificial
  primer LPXB reverse 130
ggcgcctcaatcccgacaaagacatccgg

SEQ ID NO: 141 - DNA, 29 bp, Artificial
  primer LPXB reverse 131
ggcgcctcaatcccgacaaagacatcggg

SEQ ID NO: 142 - DNA, 29 bp, Artificial
  primer LPXB reverse 132
ggcgcctcaatcccgacaaagacatctgg

SEQ ID NO: 143 - DNA, 29 bp, Artificial
  primer LPXB reverse 133
ggcgcctcaatcccgacgaaaacatcagg

SEQ ID NO: 144 - DNA, 29 bp, Artificial
  primer LPXB reverse 134
ggcgcctcaatcccgacgaaaacatccgg

SEQ ID NO: 145 - DNA, 29 bp, Artificial
  primer LPXB reverse 135
ggcgcctcaatcccgacgaaaacatcggg

SEQ ID NO: 146 - DNA, 29 bp, Artificial
  primer LPXB reverse 136
ggcgcctcaatcccgacgaaaacatctgg

SEQ ID NO: 147 - DNA, 29 bp, Artificial
  primer LPXB reverse 137
ggcgcctcaatcccgacgaacacatcagg

SEQ ID NO: 148 - DNA, 29 bp, Artificial
  primer LPXB reverse 138
ggcgcctcaatcccgacgaacacatccgg

SEQ ID NO: 149 - DNA, 29 bp, Artificial
  primer LPXB reverse 139
ggcgcctcaatcccgacgaacacatctgg

SEQ ID NO: 150 - DNA, 29 bp, Artificial
  primer LPXB reverse 140
ggcgcctcaatcccgacgaacacatcggg

SEQ ID NO: 151 - DNA, 29 bp, Artificial
  primer LPXB reverse 141
ggcgcctcaatcccgacgaagacatcagg

SEQ ID NO: 152 - DNA, 29 bp, Artificial
  primer LPXB reverse 142
ggcgcctcaatcccgacgaagacatccgg

SEQ ID NO: 153 - DNA, 29 bp, Artificial
  primer LPXB reverse 143
ggcgcctcaatcccgacgaagacatcggg

SEQ ID NO: 154 - DNA, 29 bp, Artificial
  primer LPXB reverse 144
ggcgcctcaatcccgacgaagacatctgg

SEQ ID NO: 155 - DNA, 29 bp, Artificial
  primer LPXB reverse 145
ggcgcctcaattccaacaaaaacatcagg

SEQ ID NO: 156 - DNA, 29 bp, Artificial
  primer LPXB reverse 146
ggcgcctcaattccaacaaaaacatccgg

SEQ ID NO: 157 - DNA, 29 bp, Artificial
  primer LPXB reverse 147
ggcgcctcaattccaacaaaaacatcggg

SEQ ID NO: 158 - DNA, 29 bp, Artificial
  primer LPXB reverse 148
ggcgcctcaattccaacaaaaacatctgg

SEQ ID NO: 159 - DNA, 29 bp, Artificial
  primer LPXB reverse 149
ggcgcctcaattccaacaaacacatcagg

SEQ ID NO: 160 - DNA, 29 bp, Artificial
  primer LPXB reverse 150
ggcgcctcaattccaacaaacacatccgg

SEQ ID NO: 161 - DNA, 29 bp, Artificial
  primer LPXB reverse 151
ggcgcctcaattccaacaaacacatctgg

SEQ ID NO: 162 - DNA, 29 bp, Artificial
  primer LPXB reverse 152
ggcgcctcaattccaacaaacacatcggg

SEQ ID NO: 163 - DNA, 29 bp, Artificial
  primer LPXB reverse 153
ggcgcctcaattccaacaaagacatcagg

SEQ ID NO: 164 - DNA, 29 bp, Artificial
  primer LPXB reverse 154
ggcgcctcaattccaacaaagacatccgg

SEQ ID NO: 165 - DNA, 29 bp, Artificial
  primer LPXB reverse 155
ggcgcctcaattccaacaaagacatcggg

SEQ ID NO: 166 - DNA, 29 bp, Artificial
  primer LPXB reverse 156
ggcgcctcaattccaacaaagacatctgg

SEQ ID NO: 167 - DNA, 29 bp, Artificial
  primer LPXB reverse 157
ggcgcctcaattccaacgaaaacatcagg

SEQ ID NO: 168 - DNA, 29 bp, Artificial
  primer LPXB reverse 158
ggcgcctcaattccaacgaaaacatccgg

SEQ ID NO: 169 - DNA, 29 bp, Artificial
  primer LPXB reverse 159
ggcgcctcaattccaacgaaaacatcggg

SEQ ID NO: 170 - DNA, 29 bp, Artificial
  primer LPXB reverse 160
ggcgcctcaattccaacgaaaacatctgg

SEQ ID NO: 171 - DNA, 29 bp, Artificial
  primer LPXB reverse 161
ggcgcctcaattccaacgaacacatcagg

SEQ ID NO: 172 - DNA, 29 bp, Artificial
  primer LPXB reverse 162
ggcgcctcaattccaacgaacacatccgg

SEQ ID NO: 173 - DNA, 29 bp, Artificial
  primer LPXB reverse 163
ggcgcctcaattccaacgaacacatctgg

SEQ ID NO: 174 - DNA, 29 bp, Artificial
  primer LPXB reverse 164
ggcgcctcaattccaacgaacacatcggg

SEQ ID NO: 175 - DNA, 29 bp, Artificial
  primer LPXB reverse 165
ggcgcctcaattccaacgaagacatcagg

SEQ ID NO: 176 - DNA, 29 bp, Artificial
  primer LPXB reverse 166
ggcgcctcaattccaacgaagacatccgg

SEQ ID NO: 177 - DNA, 29 bp, Artificial
  primer LPXB reverse 167
ggcgcctcaattccaacgaagacatcggg

SEQ ID NO: 178 - DNA, 29 bp, Artificial
  primer LPXB reverse 168
ggcgcctcaattccaacgaagacatctgg

SEQ ID NO: 179 - DNA, 29 bp, Artificial
  primer LPXB reverse 169
ggcgcctcaattcccacaaaaacatcagg

SEQ ID NO: 180 - DNA, 29 bp, Artificial
  primer LPXB reverse 170
ggcgcctcaattcccacaaaaacatccgg

SEQ ID NO: 181 - DNA, 29 bp, Artificial
  primer LPXB reverse 171
ggcgcctcaattcccacaaaaacatcggg

SEQ ID NO: 182 - DNA, 29 bp, Artificial
  primer LPXB reverse 172
ggcgcctcaattcccacaaaaacatctgg

SEQ ID NO: 183 - DNA, 29 bp, Artificial
  primer LPXB reverse 173
ggcgcctcaattcccacaaacacatcagg

SEQ ID NO: 184 - DNA, 29 bp, Artificial
  primer LPXB reverse 174
ggcgcctcaattcccacaaacacatccgg

SEQ ID NO: 185 - DNA, 29 bp, Artificial
  primer LPXB reverse 175
ggcgcctcaattcccacaaacacatctgg

SEQ ID NO: 186 - DNA, 29 bp, Artificial
  primer LPXB reverse 176
ggcgcctcaattcccacaaacacatcggg

SEQ ID NO: 187 - DNA, 29 bp, Artificial
  primer LPXB reverse 177
ggcgcctcaattcccacaaagacatcagg

SEQ ID NO: 188 - DNA, 29 bp, Artificial
  primer LPXB reverse 178
ggcgcctcaattcccacaaagacatccgg

SEQ ID NO: 189 - DNA, 29 bp, Artificial
  primer LPXB reverse 179
ggcgcctcaattcccacaaagacatcggg

SEQ ID NO: 190 - DNA, 29 bp, Artificial
  primer LPXB reverse 180
ggcgcctcaattcccacaaagacatctgg

SEQ ID NO: 191 - DNA, 29 bp, Artificial
  primer LPXB reverse 181
ggcgcctcaattcccacgaaaacatcagg

SEQ ID NO: 192 - DNA, 29 bp, Artificial
  primer LPXB reverse 182
ggcgcctcaattcccacgaaaacatccgg

SEQ ID NO: 193 - DNA, 29 bp, Artificial
  primer LPXB reverse 183
ggcgcctcaattcccacgaaaacatcggg

SEQ ID NO: 194 - DNA, 29 bp, Artificial
  primer LPXB reverse 184
ggcgcctcaattcccacgaaaacatctgg

SEQ ID NO: 195 - DNA, 29 bp, Artificial
  primer LPXB reverse 185
ggcgcctcaattcccacgaacacatcagg

SEQ ID NO: 196 - DNA, 29 bp, Artificial
  primer LPXB reverse 186
ggcgcctcaattcccacgaacacatccgg

SEQ ID NO: 197 - DNA, 29 bp, Artificial
  primer LPXB reverse 187
ggcgcctcaattcccacgaacacatctgg

SEQ ID NO: 198 - DNA, 29 bp, Artificial
  primer LPXB reverse 188
ggcgcctcaattcccacgaacacatcggg

SEQ ID NO: 199 - DNA, 29 bp, Artificial
  primer LPXB reverse 189
ggcgcctcaattcccacgaagacatcagg

SEQ ID NO: 200 - DNA, 29 bp, Artificial
  primer LPXB reverse 190
ggcgcctcaattcccacgaagacatccgg

SEQ ID NO: 201 - DNA, 29 bp, Artificial
  primer LPXB reverse 191
ggcgcctcaattcccacgaagacatcggg

SEQ ID NO: 202 - DNA, 29 bp, Artificial
  primer LPXB reverse 192
ggcgcctcaattcccacgaagacatctgg

SEQ ID NO: 203 - DNA, 29 bp, Artificial
  primer LPXB reverse 193
ggcgcctcaattccgacaaaaacatcagg

SEQ ID NO: 204 - DNA, 29 bp, Artificial
  primer LPXB reverse 194
ggcgcctcaattccgacaaaaacatccgg

SEQ ID NO: 205 - DNA, 29 bp, Artificial
  primer LPXB reverse 195
ggcgcctcaattccgacaaaaacatcggg

SEQ ID NO: 206 - DNA, 29 bp, Artificial
  primer LPXB reverse 196
ggcgcctcaattccgacaaaaacatctgg

SEQ ID NO: 207 - DNA, 29 bp, Artificial
  primer LPXB reverse 197
ggcgcctcaattccgacaaacacatcagg

SEQ ID NO: 208 - DNA, 29 bp, Artificial
  primer LPXB reverse 198
ggcgcctcaattccgacaaacacatccgg

SEQ ID NO: 209 - DNA, 29 bp, Artificial
  primer LPXB reverse 199
ggcgcctcaattccgacaaacacatctgg

SEQ ID NO: 210 - DNA, 29 bp, Artificial
  primer LPXB reverse 200
ggcgcctcaattccgacaaacacatcggg

SEQ ID NO: 211 - DNA, 29 bp, Artificial
  primer LPXB reverse 201
ggcgcctcaattccgacaaagacatcagg

SEQ ID NO: 212 - DNA, 29 bp, Artificial
  primer LPXB reverse 202
ggcgcctcaattccgacaaagacatccgg

SEQ ID NO: 213 - DNA, 29 bp, Artificial
  primer LPXB reverse 203
ggcgcctcaattccgacaaagacatcggg

SEQ ID NO: 214 - DNA, 29 bp, Artificial
  primer LPXB reverse 204
ggcgcctcaattccgacaaagacatctgg

SEQ ID NO: 215 - DNA, 29 bp, Artificial
  primer LPXB reverse 205
ggcgcctcaattccgacgaaaacatcagg

SEQ ID NO: 216 - DNA, 29 bp, Artificial
  primer LPXB reverse 206
ggcgcctcaattccgacgaaaacatccgg

SEQ ID NO: 217 - DNA, 29 bp, Artificial
  primer LPXB reverse 207
ggcgcctcaattccgacgaaaacatcggg

SEQ ID NO: 218 - DNA, 29 bp, Artificial
  primer LPXB reverse 208
ggcgcctcaattccgacgaaaacatctgg

SEQ ID NO: 219 - DNA, 29 bp, Artificial
  primer LPXB reverse 209
ggcgcctcaattccgacgaacacatcagg

SEQ ID NO: 220 - DNA, 29 bp, Artificial
  primer LPXB reverse 210
ggcgcctcaattccgacgaacacatccgg

SEQ ID NO: 221 - DNA, 29 bp, Artificial
  primer LPXB reverse 211
ggcgcctcaattccgacgaacacatctgg

SEQ ID NO: 222 - DNA, 29 bp, Artificial
  primer LPXB reverse 212
ggcgcctcaattccgacgaacacatcggg

SEQ ID NO: 223 - DNA, 29 bp, Artificial
  primer LPXB reverse 213
ggcgcctcaattccgacgaagacatcagg

SEQ ID NO: 224 - DNA, 29 bp, Artificial
  primer LPXB reverse 214
ggcgcctcaattccgacgaagacatccgg

SEQ ID NO: 225 - DNA, 29 bp, Artificial
  primer LPXB reverse 215
ggcgcctcaattccgacgaagacatcggg

SEQ ID NO: 226 - DNA, 29 bp, Artificial
  primer LPXB reverse 216
ggcgcctcaattccgacgaagacatctgg

SEQ ID NO: 227 - DNA, 29 bp, Artificial
  primer LPXB reverse 217
ggcgcctcgataccaacaaaaacatcagg

SEQ ID NO: 228 - DNA, 29 bp, Artificial
  primer LPXB reverse 218
ggcgcctcgataccaacaaaaacatccgg

SEQ ID NO: 229 - DNA, 29 bp, Artificial
  primer LPXB reverse 219
ggcgcctcgataccaacaaaaacatcggg

SEQ ID NO: 230 - DNA, 29 bp, Artificial
  primer LPXB reverse 220
ggcgcctcgataccaacaaaaacatctgg

SEQ ID NO: 231 - DNA, 29 bp, Artificial
  primer LPXB reverse 221
ggcgcctcgataccaacaaacacatcagg

SEQ ID NO: 232 - DNA, 29 bp, Artificial
  primer LPXB reverse 222
ggcgcctcgataccaacaaacacatccgg

SEQ ID NO: 233 - DNA, 29 bp, Artificial
  primer LPXB reverse 223
ggcgcctcgataccaacaaacacatctgg

SEQ ID NO: 234 - DNA, 29 bp, Artificial
  primer LPXB reverse 224
ggcgcctcgataccaacaaacacatcggg

SEQ ID NO: 235 - DNA, 29 bp, Artificial
  primer LPXB reverse 225
ggcgcctcgataccaacaaagacatcagg

SEQ ID NO: 236 - DNA, 29 bp, Artificial
  primer LPXB reverse 226
ggcgcctcgataccaacaaagacatccgg

SEQ ID NO: 237 - DNA, 29 bp, Artificial
  primer LPXB reverse 227
ggcgcctcgataccaacaaagacatcggg

SEQ ID NO: 238 - DNA, 29 bp, Artificial
  primer LPXB reverse 228
ggcgcctcgataccaacaaagacatctgg

SEQ ID NO: 239 - DNA, 29 bp, Artificial
  primer LPXB reverse 229
ggcgcctcgataccaacgaaaacatcagg

SEQ ID NO: 240 - DNA, 29 bp, Artificial
  primer LPXB reverse 230
ggcgcctcgataccaacgaaaacatccgg

SEQ ID NO: 241 - DNA, 29 bp, Artificial
  primer LPXB reverse 231
ggcgcctcgataccaacgaaaacatcggg

SEQ ID NO: 242 - DNA, 29 bp, Artificial
  primer LPXB reverse 232
ggcgcctcgataccaacgaaaacatctgg

SEQ ID NO: 243 - DNA, 29 bp, Artificial
  primer LPXB reverse 233
ggcgcctcgataccaacgaacacatcagg

SEQ ID NO: 244 - DNA, 29 bp, Artificial
  primer LPXB reverse 234
ggcgcctcgataccaacgaacacatccgg

SEQ ID NO: 245 - DNA, 29 bp, Artificial
  primer LPXB reverse 235
ggcgcctcgataccaacgaacacatctgg

SEQ ID NO: 246 - DNA, 29 bp, Artificial
  primer LPXB reverse 236
ggcgcctcgataccaacgaacacatcggg

SEQ ID NO: 247 - DNA, 29 bp, Artificial
  primer LPXB reverse 237
ggcgcctcgataccaacgaagacatcagg

SEQ ID NO: 248 - DNA, 29 bp, Artificial
  primer LPXB reverse 238
ggcgcctcgataccaacgaagacatccgg

SEQ ID NO: 249 - DNA, 29 bp, Artificial
  primer LPXB reverse 239
ggcgcctcgataccaacgaagacatcggg

SEQ ID NO: 250 - DNA, 29 bp, Artificial
  primer LPXB reverse 240
ggcgcctcgataccaacgaagacatctgg

SEQ ID NO: 251 - DNA, 29 bp, Artificial
  primer LPXB reverse 241
ggcgcctcgatacccacaaaaacatcagg

SEQ ID NO: 252 - DNA, 29 bp, Artificial
  primer LPXB reverse 242
ggcgcctcgatacccacaaaaacatccgg

SEQ ID NO: 253 - DNA, 29 bp, Artificial
  primer LPXB reverse 243
ggcgcctcgatacccacaaaaacatcggg

SEQ ID NO: 254 - DNA, 29 bp, Artificial
  primer LPXB reverse 244
ggcgcctcgatacccacaaaaacatctgg

SEQ ID NO: 255 - DNA, 29 bp, Artificial
  primer LPXB reverse 245
ggcgcctcgatacccacaaacacatcagg

SEQ ID NO: 256 - DNA, 29 bp, Artificial
  primer LPXB reverse 246
ggcgcctcgatacccacaaacacatccgg

SEQ ID NO: 257 - DNA, 29 bp, Artificial
  primer LPXB reverse 247
ggcgcctcgatacccacaaacacatctgg

SEQ ID NO: 258 - DNA, 29 bp, Artificial
  primer LPXB reverse 248
ggcgcctcgatacccacaaacacatcggg

SEQ ID NO: 259 - DNA, 29 bp, Artificial
  primer LPXB reverse 249
ggcgcctcgatacccacaaagacatcagg

SEQ ID NO: 260 - DNA, 29 bp, Artificial
  primer LPXB reverse 250
ggcgcctcgatacccacaaagacatccgg

SEQ ID NO: 261 - DNA, 29 bp, Artificial
  primer LPXB reverse 251
ggcgcctcgatacccacaaagacatcggg

SEQ ID NO: 262 - DNA, 29 bp, Artificial
  primer LPXB reverse 252
ggcgcctcgatacccacaaagacatctgg

SEQ ID NO: 263 - DNA, 29 bp, Artificial
  primer LPXB reverse 253
ggcgcctcgatacccacgaaaacatcagg

SEQ ID NO: 264 - DNA, 29 bp, Artificial
  primer LPXB reverse 254
ggcgcctcgatacccacgaaaacatccgg

SEQ ID NO: 265 - DNA, 29 bp, Artificial
  primer LPXB reverse 255
ggcgcctcgatacccacgaaaacatcggg

SEQ ID NO: 266 - DNA, 29 bp, Artificial
  primer LPXB reverse 256
ggcgcctcgatacccacgaaaacatctgg

SEQ ID NO: 267 - DNA, 29 bp, Artificial
  primer LPXB reverse 257
ggcgcctcgatacccacgaacacatcagg

SEQ ID NO: 268 - DNA, 29 bp, Artificial
  primer LPXB reverse 258
ggcgcctcgatacccacgaacacatccgg

SEQ ID NO: 269 - DNA, 29 bp, Artificial
  primer LPXB reverse 259
ggcgcctcgatacccacgaacacatctgg

SEQ ID NO: 270 - DNA, 29 bp, Artificial
  primer LPXB reverse 260
ggcgcctcgatacccacgaacacatcggg

SEQ ID NO: 271 - DNA, 29 bp, Artificial
  primer LPXB reverse 261
ggcgcctcgatacccacgaagacatcagg

SEQ ID NO: 272 - DNA, 29 bp, Artificial
  primer LPXB reverse 262
ggcgcctcgatacccacgaagacatccgg

SEQ ID NO: 273 - DNA, 29 bp, Artificial
  primer LPXB reverse 263
ggcgcctcgatacccacgaagacatcggg

SEQ ID NO: 274 - DNA, 29 bp, Artificial
  primer LPXB reverse 264
ggcgcctcgatacccacgaagacatctgg

SEQ ID NO: 275 - DNA, 29 bp, Artificial
  primer LPXB reverse 265
ggcgcctcgataccgacaaaaacatcagg

SEQ ID NO: 276 - DNA, 29 bp, Artificial
  primer LPXB reverse 266
ggcgcctcgataccgacaaaaacatccgg

SEQ ID NO: 277 - DNA, 29 bp, Artificial
  primer LPXB reverse 267
ggcgcctcgataccgacaaaaacatcggg

SEQ ID NO: 278 - DNA, 29 bp, Artificial
  primer LPXB reverse 268
ggcgcctcgataccgacaaaaacatctgg

SEQ ID NO: 279 - DNA, 29 bp, Artificial
  primer LPXB reverse 269
ggcgcctcgataccgacaaacacatcagg

SEQ ID NO: 280 - DNA, 29 bp, Artificial
  primer LPXB reverse 270
ggcgcctcgataccgacaaacacatccgg

SEQ ID NO: 281 - DNA, 29 bp, Artificial
  primer LPXB reverse 271
ggcgcctcgataccgacaaacacatctgg

SEQ ID NO: 282 - DNA, 29 bp, Artificial
  primer LPXB reverse 272
ggcgcctcgataccgacaaacacatcggg

SEQ ID NO: 283 - DNA, 29 bp, Artificial
  primer LPXB reverse 273
ggcgcctcgataccgacaaagacatcagg

SEQ ID NO: 284 - DNA, 29 bp, Artificial
  primer LPXB reverse 274
ggcgcctcgataccgacaaagacatccgg

SEQ ID NO: 285 - DNA, 29 bp, Artificial
  primer LPXB reverse 275
ggcgcctcgataccgacaaagacatcggg

SEQ ID NO: 286 - DNA, 29 bp, Artificial
  primer LPXB reverse 276
ggcgcctcgataccgacaaagacatctgg

SEQ ID NO: 287 - DNA, 29 bp, Artificial
  primer LPXB reverse 277
ggcgcctcgataccgacgaaaacatcagg

SEQ ID NO: 288 - DNA, 29 bp, Artificial
  primer LPXB reverse 278
ggcgcctcgataccgacgaaaacatccgg

SEQ ID NO: 289 - DNA, 29 bp, Artificial
  primer LPXB reverse 279
ggcgcctcgataccgacgaaaacatcggg

SEQ ID NO: 290 - DNA, 29 bp, Artificial
  primer LPXB reverse 280
ggcgcctcgataccgacgaaaacatctgg

SEQ ID NO: 291 - DNA, 29 bp, Artificial
  primer LPXB reverse 281
ggcgcctcgataccgacgaacacatcagg

SEQ ID NO: 292 - DNA, 29 bp, Artificial
  primer LPXB reverse 282
ggcgcctcgataccgacgaacacatccgg

SEQ ID NO: 293 - DNA, 29 bp, Artificial
  primer LPXB reverse 283
ggcgcctcgataccgacgaacacatctgg

SEQ ID NO: 294 - DNA, 29 bp, Artificial
  primer LPXB reverse 284
ggcgcctcgataccgacgaacacatcggg

SEQ ID NO: 295 - DNA, 29 bp, Artificial
  primer LPXB reverse 285
ggcgcctcgataccgacgaagacatcagg

SEQ ID NO: 296 - DNA, 29 bp, Artificial
  primer LPXB reverse 286
ggcgcctcgataccgacgaagacatccgg

SEQ ID NO: 297 - DNA, 29 bp, Artificial
  primer LPXB reverse 287
ggcgcctcgataccgacgaagacatcggg

SEQ ID NO: 298 - DNA, 29 bp, Artificial
  primer LPXB reverse 288
ggcgcctcgataccgacgaagacatctgg

SEQ ID NO: 299 - DNA, 29 bp, Artificial
  primer LPXB reverse 289
ggcgcctcgatcccaacaaaaacatcagg

SEQ ID NO: 300 - DNA, 29 bp, Artificial
  primer LPXB reverse 290
ggcgcctcgatcccaacaaaaacatccgg

SEQ ID NO: 301 - DNA, 29 bp, Artificial
  primer LPXB reverse 291
ggcgcctcgatcccaacaaaaacatcggg

SEQ ID NO: 302 - DNA, 29 bp, Artificial
  primer LPXB reverse 292
ggcgcctcgatcccaacaaaaacatctgg

SEQ ID NO: 303 - DNA, 29 bp, Artificial
  primer LPXB reverse 293
ggcgcctcgatcccaacaaacacatcagg

SEQ ID NO: 304 - DNA, 29 bp, Artificial
  primer LPXB reverse 294
ggcgcctcgatcccaacaaacacatccgg

SEQ ID NO: 305 - DNA, 29 bp, Artificial
  primer LPXB reverse 295
ggcgcctcgatcccaacaaacacatctgg

SEQ ID NO: 306 - DNA, 29 bp, Artificial
  primer LPXB reverse 296
ggcgcctcgatcccaacaaacacatcggg

SEQ ID NO: 307 - DNA, 29 bp, Artificial
  primer LPXB reverse 297
ggcgcctcgatcccaacaaagacatcagg

SEQ ID NO: 308 - DNA, 29 bp, Artificial
  primer LPXB reverse 298
ggcgcctcgatcccaacaaagacatccgg

SEQ ID NO: 309 - DNA, 29 bp, Artificial
  primer LPXB reverse 299
ggcgcctcgatcccaacaaagacatcggg

SEQ ID NO: 310 - DNA, 29 bp, Artificial
  primer LPXB reverse 300
ggcgcctcgatcccaacaaagacatctgg

SEQ ID NO: 311 - DNA, 29 bp, Artificial
  primer LPXB reverse 301
ggcgcctcgatcccaacgaaaacatcagg

SEQ ID NO: 312 - DNA, 29 bp, Artificial
  primer LPXB reverse 302
ggcgcctcgatcccaacgaaaacatccgg

SEQ ID NO: 313 - DNA, 29 bp, Artificial
  primer LPXB reverse 303
ggcgcctcgatcccaacgaaaacatcggg

SEQ ID NO: 314 - DNA, 29 bp, Artificial
  primer LPXB reverse 304
ggcgcctcgatcccaacgaaaacatctgg

SEQ ID NO: 315 - DNA, 29 bp, Artificial
  primer LPXB reverse 305
ggcgcctcgatcccaacgaacacatcagg

SEQ ID NO: 316 - DNA, 29 bp, Artificial
  primer LPXB reverse 306
ggcgcctcgatcccaacgaacacatccgg

SEQ ID NO: 317 - DNA, 29 bp, Artificial
  primer LPXB reverse 307
ggcgcctcgatcccaacgaacacatctgg

SEQ ID NO: 318 - DNA, 29 bp, Artificial
  primer LPXB reverse 308
ggcgcctcgatcccaacgaacacatcggg

SEQ ID NO: 319 - DNA, 29 bp, Artificial
  primer LPXB reverse 309
ggcgcctcgatcccaacgaagacatcagg

SEQ ID NO: 320 - DNA, 29 bp, Artificial
  primer LPXB reverse 310
ggcgcctcgatcccaacgaagacatccgg

SEQ ID NO: 321 - DNA, 29 bp, Artificial
  primer LPXB reverse 311
ggcgcctcgatcccaacgaagacatcggg

SEQ ID NO: 322 - DNA, 29 bp, Artificial
  primer LPXB reverse 312
ggcgcctcgatcccaacgaagacatctgg

SEQ ID NO: 323 - DNA, 29 bp, Artificial
  primer LPXB reverse 313
ggcgcctcgatccccacaaaaacatcagg

SEQ ID NO: 324 - DNA, 29 bp, Artificial
  primer LPXB reverse 314
ggcgcctcgatccccacaaaaacatccgg

SEQ ID NO: 325 - DNA, 29 bp, Artificial
  primer LPXB reverse 315
ggcgcctcgatccccacaaaaacatcggg

SEQ ID NO: 326 - DNA, 29 bp, Artificial
  primer LPXB reverse 316
ggcgcctcgatccccacaaaaacatctgg

SEQ ID NO: 327 - DNA, 29 bp, Artificial
  primer LPXB reverse 317
ggcgcctcgatccccacaaacacatcagg

SEQ ID NO: 328 - DNA, 29 bp, Artificial
  primer LPXB reverse 318
ggcgcctcgatccccacaaacacatccgg

SEQ ID NO: 329 - DNA, 29 bp, Artificial
  primer LPXB reverse 319
ggcgcctcgatccccacaaacacatctgg

SEQ ID NO: 330 - DNA, 29 bp, Artificial
  primer LPXB reverse 320
ggcgcctcgatccccacaaacacatcggg

SEQ ID NO: 331 - DNA, 29 bp, Artificial
  primer LPXB reverse 321
ggcgcctcgatccccacaaagacatcagg

SEQ ID NO: 332 - DNA, 29 bp, Artificial
  primer LPXB reverse 322
ggcgcctcgatccccacaaagacatccgg

SEQ ID NO: 333 - DNA, 29 bp, Artificial
  primer LPXB reverse 323
ggcgcctcgatccccacaaagacatcggg

SEQ ID NO: 334 - DNA, 29 bp, Artificial
  primer LPXB reverse 324
ggcgcctcgatccccacaaagacatctgg

SEQ ID NO: 335 - DNA, 29 bp, Artificial
  primer LPXB reverse 325
ggcgcctcgatccccacgaaaacatcagg

SEQ ID NO: 336 - DNA, 29 bp, Artificial
  primer LPXB reverse 326
ggcgcctcgatccccacgaaaacatccgg

SEQ ID NO: 337 - DNA, 29 bp, Artificial
  primer LPXB reverse 327
ggcgcctcgatccccacgaaaacatcggg

SEQ ID NO: 338 - DNA, 29 bp, Artificial
  primer LPXB reverse 328
ggcgcctcgatccccacgaaaacatctgg

SEQ ID NO: 339 - DNA, 29 bp, Artificial
  primer LPXB reverse 329
ggcgcctcgatccccacgaacacatcagg

SEQ ID NO: 340 - DNA, 29 bp, Artificial
  primer LPXB reverse 330
ggcgcctcgatccccacgaacacatccgg

SEQ ID NO: 341 - DNA, 29 bp, Artificial
  primer LPXB reverse 331
ggcgcctcgatccccacgaacacatctgg

SEQ ID NO: 342 - DNA, 29 bp, Artificial
  primer LPXB reverse 332
ggcgcctcgatccccacgaacacatcggg

SEQ ID NO: 343 - DNA, 29 bp, Artificial
  primer LPXB reverse 333
ggcgcctcgatccccacgaagacatcagg

SEQ ID NO: 344 - DNA, 29 bp, Artificial
  primer LPXB reverse 334
ggcgcctcgatccccacgaagacatccgg

SEQ ID NO: 345 - DNA, 29 bp, Artificial
  primer LPXB reverse 335
ggcgcctcgatccccacgaagacatcggg

SEQ ID NO: 346 - DNA, 29 bp, Artificial
  primer LPXB reverse 336
ggcgcctcgatccccacgaagacatctgg

SEQ ID NO: 347 - DNA, 29 bp, Artificial
  primer LPXB reverse 337
ggcgcctcgatcccgacaaaaacatcagg

SEQ ID NO: 348 - DNA, 29 bp, Artificial
  primer LPXB reverse 338
ggcgcctcgatcccgacaaaaacatccgg

SEQ ID NO: 349 - DNA, 29 bp, Artificial
  primer LPXB reverse 339
ggcgcctcgatcccgacaaaaacatcggg

SEQ ID NO: 350 - DNA, 29 bp, Artificial
  primer LPXB reverse 340
ggcgcctcgatcccgacaaaaacatctgg

SEQ ID NO: 351 - DNA, 29 bp, Artificial
  primer LPXB reverse 341
ggcgcctcgatcccgacaaacacatcagg

SEQ ID NO: 352 - DNA, 29 bp, Artificial
  primer LPXB reverse 342
ggcgcctcgatcccgacaaacacatccgg

SEQ ID NO: 353 - DNA, 29 bp, Artificial
  primer LPXB reverse 343
ggcgcctcgatcccgacaaacacatctgg

SEQ ID NO: 354 - DNA, 29 bp, Artificial
  primer LPXB reverse 344
ggcgcctcgatcccgacaaacacatcggg

SEQ ID NO: 355 - DNA, 29 bp, Artificial
  primer LPXB reverse 345
ggcgcctcgatcccgacaaagacatcagg

SEQ ID NO: 356 - DNA, 29 bp, Artificial
  primer LPXB reverse 346
ggcgcctcgatcccgacaaagacatccgg

SEQ ID NO: 357 - DNA, 29 bp, Artificial
  primer LPXB reverse 347
ggcgcctcgatcccgacaaagacatcggg

SEQ ID NO: 358 - DNA, 29 bp, Artificial
  primer LPXB reverse 348
ggcgcctcgatcccgacaaagacatctgg

SEQ ID NO: 359 - DNA, 29 bp, Artificial
  primer LPXB reverse 349
ggcgcctcgatcccgacgaaaacatcagg

SEQ ID NO: 360 - DNA, 29 bp, Artificial
  primer LPXB reverse 350
ggcgcctcgatcccgacgaaaacatccgg

SEQ ID NO: 361 - DNA, 29 bp, Artificial
  primer LPXB reverse 351
ggcgcctcgatcccgacgaaaacatcggg

SEQ ID NO: 362 - DNA, 29 bp, Artificial
  primer LPXB reverse 352
ggcgcctcgatcccgacgaaaacatctgg

SEQ ID NO: 363 - DNA, 29 bp, Artificial
  primer LPXB reverse 353
ggcgcctcgatcccgacgaacacatcagg

SEQ ID NO: 364 - DNA, 29 bp, Artificial
  primer LPXB reverse 354
ggcgcctcgatcccgacgaacacatccgg

SEQ ID NO: 365 - DNA, 29 bp, Artificial
  primer LPXB reverse 355
ggcgcctcgatcccgacgaacacatctgg

SEQ ID NO: 366 - DNA, 29 bp, Artificial
  primer LPXB reverse 356
ggcgcctcgatcccgacgaacacatcggg

SEQ ID NO: 367 - DNA, 29 bp, Artificial
  primer LPXB reverse 357
ggcgcctcgatcccgacgaagacatcagg

SEQ ID NO: 368 - DNA, 29 bp, Artificial
  primer LPXB reverse 358
ggcgcctcgatcccgacgaagacatccgg

SEQ ID NO: 369 - DNA, 29 bp, Artificial
  primer LPXB reverse 359
ggcgcctcgatcccgacgaagacatcggg

SEQ ID NO: 370 - DNA, 29 bp, Artificial
  primer LPXB reverse 360
ggcgcctcgatcccgacgaagacatctgg

SEQ ID NO: 371 - DNA, 29 bp, Artificial
  primer LPXB reverse 361
ggcgcctcgattccaacaaaaacatcagg

SEQ ID NO: 372 - DNA, 29 bp, Artificial
  primer LPXB reverse 362
ggcgcctcgattccaacaaaaacatccgg

SEQ ID NO: 373 - DNA, 29 bp, Artificial
  primer LPXB reverse 363
ggcgcctcgattccaacaaaaacatcggg

SEQ ID NO: 374 - DNA, 29 bp, Artificial
  primer LPXB reverse 364
ggcgcctcgattccaacaaaaacatctgg

SEQ ID NO: 375 - DNA, 29 bp, Artificial
  primer LPXB reverse 365
ggcgcctcgattccaacaaacacatcagg

SEQ ID NO: 376 - DNA, 29 bp, Artificial
  primer LPXB reverse 366
ggcgcctcgattccaacaaacacatccgg

SEQ ID NO: 377 - DNA, 29 bp, Artificial
  primer LPXB reverse 367
ggcgcctcgattccaacaaacacatctgg

SEQ ID NO: 378 - DNA, 29 bp, Artificial
  primer LPXB reverse 368
ggcgcctcgattccaacaaacacatcggg

SEQ ID NO: 379 - DNA, 29 bp, Artificial
  primer LPXB reverse 369
ggcgcctcgattccaacaaagacatcagg

SEQ ID NO: 380 - DNA, 29 bp, Artificial
  primer LPXB reverse 370
ggcgcctcgattccaacaaagacatccgg

SEQ ID NO: 381 - DNA, 29 bp, Artificial
  primer LPXB reverse 371
ggcgcctcgattccaacaaagacatcggg

SEQ ID NO: 382 - DNA, 29 bp, Artificial
  primer LPXB reverse 372
ggcgcctcgattccaacaaagacatctgg

SEQ ID NO: 383 - DNA, 29 bp, Artificial
  primer LPXB reverse 373
ggcgcctcgattccaacgaaaacatcagg

SEQ ID NO: 384 - DNA, 29 bp, Artificial
  primer LPXB reverse 374
ggcgcctcgattccaacgaaaacatccgg

SEQ ID NO: 385 - DNA, 29 bp, Artificial
  primer LPXB reverse 375
ggcgcctcgattccaacgaaaacatcggg

SEQ ID NO: 386 - DNA, 29 bp, Artificial
  primer LPXB reverse 376
ggcgcctcgattccaacgaaaacatctgg

SEQ ID NO: 387 - DNA, 29 bp, Artificial
  primer LPXB reverse 377
ggcgcctcgattccaacgaacacatcagg

SEQ ID NO: 388 - DNA, 29 bp, Artificial
  primer LPXB reverse 378
ggcgcctcgattccaacgaacacatccgg

SEQ ID NO: 389 - DNA, 29 bp, Artificial
  primer LPXB reverse 379
ggcgcctcgattccaacgaacacatctgg

SEQ ID NO: 390 - DNA, 29 bp, Artificial
  primer LPXB reverse 380
ggcgcctcgattccaacgaacacatcggg

SEQ ID NO: 391 - DNA, 29 bp, Artificial
  primer LPXB reverse 381
ggcgcctcgattccaacgaagacatcagg

SEQ ID NO: 392 - DNA, 29 bp, Artificial
  primer LPXB reverse 382
ggcgcctcgattccaacgaagacatccgg

SEQ ID NO: 393 - DNA, 29 bp, Artificial
  primer LPXB reverse 383
ggcgcctcgattccaacgaagacatcggg

SEQ ID NO: 394 - DNA, 29 bp, Artificial
  primer LPXB reverse 384
ggcgcctcgattccaacgaagacatctgg

SEQ ID NO: 395 - DNA, 29 bp, Artificial
  primer LPXB reverse 385
ggcgcctcgattcccacaaaaacatcagg

SEQ ID NO: 396 - DNA, 29 bp, Artificial
  primer LPXB reverse 386
ggcgcctcgattcccacaaaaacatccgg

SEQ ID NO: 397 - DNA, 29 bp, Artificial
  primer LPXB reverse 387
ggcgcctcgattcccacaaaaacatcggg

SEQ ID NO: 398 - DNA, 29 bp, Artificial
  primer LPXB reverse 388
ggcgcctcgattcccacaaaaacatctgg

SEQ ID NO: 399 - DNA, 29 bp, Artificial
  primer LPXB reverse 389
ggcgcctcgattcccacaaacacatcagg

SEQ ID NO: 400 - DNA, 29 bp, Artificial
  primer LPXB reverse 390
ggcgcctcgattcccacaaacacatccgg

SEQ ID NO: 401 - DNA, 29 bp, Artificial
  primer LPXB reverse 391
ggcgcctcgattcccacaaacacatctgg

SEQ ID NO: 402 - DNA, 29 bp, Artificial
  primer LPXB reverse 392
ggcgcctcgattcccacaaacacatcggg

SEQ ID NO: 403 - DNA, 29 bp, Artificial
  primer LPXB reverse 393
ggcgcctcgattcccacaaagacatcagg

SEQ ID NO: 404 - DNA, 29 bp, Artificial
  primer LPXB reverse 394
ggcgcctcgattcccacaaagacatccgg

SEQ ID NO: 405 - DNA, 29 bp, Artificial
  primer LPXB reverse 395
ggcgcctcgattcccacaaagacatcggg

SEQ ID NO: 406 - DNA, 29 bp, Artificial
  primer LPXB reverse 396
ggcgcctcgattcccacaaagacatctgg

SEQ ID NO: 407 - DNA, 29 bp, Artificial
  primer LPXB reverse 397
ggcgcctcgattcccacgaaaacatcagg

SEQ ID NO: 408 - DNA, 29 bp, Artificial
  primer LPXB reverse 398
ggcgcctcgattcccacgaaaacatccgg

SEQ ID NO: 409 - DNA, 29 bp, Artificial
  primer LPXB reverse 399
ggcgcctcgattcccacgaaaacatcggg

SEQ ID NO: 410 - DNA, 29 bp, Artificial
  primer LPXB reverse 400
ggcgcctcgattcccacgaaaacatctgg

SEQ ID NO: 411 - DNA, 29 bp, Artificial
  primer LPXB reverse 401
ggcgcctcgattcccacgaacacatcagg

SEQ ID NO: 412 - DNA, 29 bp, Artificial
  primer LPXB reverse 402
ggcgcctcgattcccacgaacacatccgg

SEQ ID NO: 413 - DNA, 29 bp, Artificial
  primer LPXB reverse 403
ggcgcctcgattcccacgaacacatctgg

SEQ ID NO: 414 - DNA, 29 bp, Artificial
  primer LPXB reverse 404
ggcgcctcgattcccacgaacacatcggg

SEQ ID NO: 415 - DNA, 29 bp, Artificial
  primer LPXB reverse 405
ggcgcctcgattcccacgaagacatcagg

SEQ ID NO: 416 - DNA, 29 bp, Artificial
  primer LPXB reverse 406
ggcgcctcgattcccacgaagacatccgg

SEQ ID NO: 417 - DNA, 29 bp, Artificial
  primer LPXB reverse 407
ggcgcctcgattcccacgaagacatcggg

SEQ ID NO: 418 - DNA, 29 bp, Artificial
  primer LPXB reverse 408
ggcgcctcgattcccacgaagacatctgg

SEQ ID NO: 419 - DNA, 29 bp, Artificial
  primer LPXB reverse 409
ggcgcctcgattccgacaaaaacatcagg

SEQ ID NO: 420 - DNA, 29 bp, Artificial
  primer LPXB reverse 410
ggcgcctcgattccgacaaaaacatccgg

SEQ ID NO: 421 - DNA, 29 bp, Artificial
  primer LPXB reverse 411
ggcgcctcgattccgacaaaaacatcggg

SEQ ID NO: 422 - DNA, 29 bp, Artificial
  primer LPXB reverse 412
ggcgcctcgattccgacaaaaacatctgg

SEQ ID NO: 423 - DNA, 29 bp, Artificial
  primer LPXB reverse 413
ggcgcctcgattccgacaaacacatcagg

SEQ ID NO: 424 - DNA, 29 bp, Artificial
  primer LPXB reverse 414
ggcgcctcgattccgacaaacacatccgg

SEQ ID NO: 425 - DNA, 29 bp, Artificial
  primer LPXB reverse 415
ggcgcctcgattccgacaaacacatctgg

SEQ ID NO: 426 - DNA, 29 bp, Artificial
  primer LPXB reverse 416
ggcgcctcgattccgacaaacacatcggg

SEQ ID NO: 427 - DNA, 29 bp, Artificial
  primer LPXB reverse 417
ggcgcctcgattccgacaaagacatcagg

SEQ ID NO: 428 - DNA, 29 bp, Artificial
  primer LPXB reverse 418
ggcgcctcgattccgacaaagacatccgg

SEQ ID NO: 429 - DNA, 29 bp, Artificial
  primer LPXB reverse 419
ggcgcctcgattccgacaaagacatcggg

SEQ ID NO: 430 - DNA, 29 bp, Artificial
  primer LPXB reverse 420
ggcgcctcgattccgacaaagacatctgg

SEQ ID NO: 431 - DNA, 29 bp, Artificial
  primer LPXB reverse 421
ggcgcctcgattccgacgaaaacatcagg

SEQ ID NO: 432 - DNA, 29 bp, Artificial
  primer LPXB reverse 422
ggcgcctcgattccgacgaaaacatccgg

SEQ ID NO: 433 - DNA, 29 bp, Artificial
  primer LPXB reverse 423
ggcgcctcgattccgacgaaaacatcggg

SEQ ID NO: 434 - DNA, 29 bp, Artificial
  primer LPXB reverse 424
ggcgcctcgattccgacgaaaacatctgg

SEQ ID NO: 435 - DNA, 29 bp, Artificial
  primer LPXB reverse 425
ggcgcctcgattccgacgaacacatcagg

SEQ ID NO: 436 - DNA, 29 bp, Artificial
  primer LPXB reverse 426
ggcgcctcgattccgacgaacacatccgg

SEQ ID NO: 437 - DNA, 29 bp, Artificial
  primer LPXB reverse 427
ggcgcctcgattccgacgaacacatctgg

SEQ ID NO: 438 - DNA, 29 bp, Artificial
  primer LPXB reverse 428
ggcgcctcgattccgacgaacacatcggg

SEQ ID NO: 439 - DNA, 29 bp, Artificial
  primer LPXB reverse 429
ggcgcctcgattccgacgaagacatcagg

SEQ ID NO: 440 - DNA, 29 bp, Artificial
  primer LPXB reverse 430
ggcgcctcgattccgacgaagacatccgg

SEQ ID NO: 441 - DNA, 29 bp, Artificial
  primer LPXB reverse 431
ggcgcctcgattccgacgaagacatcggg

SEQ ID NO: 442 - DNA, 29 bp, Artificial
  primer LPXB reverse 432
ggcgcctcgattccgacgaagacatctgg

SEQ ID NO: 443 - DNA, 29 bp, Artificial
  primer LPXB reverse 433
ggcgcttcaataccaacaaaaacatcagg

SEQ ID NO: 444 - DNA, 29 bp, Artificial
  primer LPXB reverse 434
ggcgcttcaataccaacaaaaacatccgg

SEQ ID NO: 445 - DNA, 29 bp, Artificial
  primer LPXB reverse 435
ggcgcttcaataccaacaaaaacatcggg

SEQ ID NO: 446 - DNA, 29 bp, Artificial
  primer LPXB reverse 436
ggcgcttcaataccaacaaaaacatctgg

SEQ ID NO: 447 - DNA, 29 bp, Artificial
  primer LPXB reverse 437
ggcgcttcaataccaacaaacacatcagg

SEQ ID NO: 448 - DNA, 29 bp, Artificial
  primer LPXB reverse 438
ggcgcttcaataccaacaaacacatccgg

SEQ ID NO: 449 - DNA, 29 bp, Artificial
  primer LPXB reverse 439
ggcgcttcaataccaacaaacacatctgg

SEQ ID NO: 450 - DNA, 29 bp, Artificial
  primer LPXB reverse 440
ggcgcttcaataccaacaaacacatcggg

SEQ ID NO: 451 - DNA, 29 bp, Artificial
  primer LPXB reverse 441
ggcgcttcaataccaacaaagacatcagg

SEQ ID NO: 452 - DNA, 29 bp, Artificial
  primer LPXB reverse 442
ggcgcttcaataccaacaaagacatccgg

SEQ ID NO: 453 - DNA, 29 bp, Artificial
  primer LPXB reverse 443
ggcgcttcaataccaacaaagacatcggg

SEQ ID NO: 454 - DNA, 29 bp, Artificial
  primer LPXB reverse 444
ggcgcttcaataccaacaaagacatctgg

SEQ ID NO: 455 - DNA, 29 bp, Artificial
  primer LPXB reverse 445
ggcgcttcaataccaacgaaaacatcagg

SEQ ID NO: 456 - DNA, 29 bp, Artificial
  primer LPXB reverse 446
ggcgcttcaataccaacgaaaacatccgg

SEQ ID NO: 457 - DNA, 29 bp, Artificial
  primer LPXB reverse 447
ggcgcttcaataccaacgaaaacatcggg

SEQ ID NO: 458 - DNA, 29 bp, Artificial
  primer LPXB reverse 448
ggcgcttcaataccaacgaaaacatctgg

SEQ ID NO: 459 - DNA, 29 bp, Artificial
  primer LPXB reverse 449
ggcgcttcaataccaacgaacacatcagg

SEQ ID NO: 460 - DNA, 29 bp, Artificial
  primer LPXB reverse 450
ggcgcttcaataccaacgaacacatccgg

SEQ ID NO: 461 - DNA, 29 bp, Artificial
  primer LPXB reverse 451
ggcgcttcaataccaacgaacacatctgg

SEQ ID NO: 462 - DNA, 29 bp, Artificial
  primer LPXB reverse 452
ggcgcttcaataccaacgaacacatcggg

SEQ ID NO: 463 - DNA, 29 bp, Artificial
  primer LPXB reverse 453
ggcgcttcaataccaacgaagacatcagg

SEQ ID NO: 464 - DNA, 29 bp, Artificial
  primer LPXB reverse 454
ggcgcttcaataccaacgaagacatccgg

SEQ ID NO: 465 - DNA, 29 bp, Artificial
  primer LPXB reverse 455
ggcgcttcaataccaacgaagacatcggg

SEQ ID NO: 466 - DNA, 29 bp, Artificial
  primer LPXB reverse 456
ggcgcttcaataccaacgaagacatctgg

SEQ ID NO: 467 - DNA, 29 bp, Artificial
  primer LPXB reverse 457
ggcgcttcaatacccacaaaaacatcagg

SEQ ID NO: 468 - DNA, 29 bp, Artificial
  primer LPXB reverse 458
ggcgcttcaatacccacaaaaacatccgg

SEQ ID NO: 469 - DNA, 29 bp, Artificial
  primer LPXB reverse 459
ggcgcttcaatacccacaaaaacatcggg

SEQ ID NO: 470 - DNA, 29 bp, Artificial
  primer LPXB reverse 460
ggcgcttcaatacccacaaaaacatctgg

SEQ ID NO: 471 - DNA, 29 bp, Artificial
  primer LPXB reverse 461
ggcgcttcaatacccacaaacacatcagg

SEQ ID NO: 472 - DNA, 29 bp, Artificial
  primer LPXB reverse 462
ggcgcttcaatacccacaaacacatccgg

SEQ ID NO: 473 - DNA, 29 bp, Artificial
  primer LPXB reverse 463
ggcgcttcaatacccacaaacacatctgg

SEQ ID NO: 474 - DNA, 29 bp, Artificial
  primer LPXB reverse 464
ggcgcttcaatacccacaaacacatcggg

SEQ ID NO: 475 - DNA, 29 bp, Artificial
  primer LPXB reverse 465
ggcgcttcaatacccacaaagacatcagg

SEQ ID NO: 476 - DNA, 29 bp, Artificial
  primer LPXB reverse 466
ggcgcttcaatacccacaaagacatccgg

SEQ ID NO: 477 - DNA, 29 bp, Artificial
  primer LPXB reverse 467
ggcgcttcaatacccacaaagacatcggg

SEQ ID NO: 478 - DNA, 29 bp, Artificial
  primer LPXB reverse 468
ggcgcttcaatacccacaaagacatctgg

SEQ ID NO: 479 - DNA, 29 bp, Artificial
  primer LPXB reverse 469
ggcgcttcaatacccacgaaaacatcagg

SEQ ID NO: 480 - DNA, 29 bp, Artificial
  primer LPXB reverse 470
ggcgcttcaatacccacgaaaacatccgg

SEQ ID NO: 481 - DNA, 29 bp, Artificial
  primer LPXB reverse 471
ggcgcttcaatacccacgaaaacatcggg

SEQ ID NO: 482 - DNA, 29 bp, Artificial
  primer LPXB reverse 472
ggcgcttcaatacccacgaaaacatctgg

SEQ ID NO: 483 - DNA, 29 bp, Artificial
  primer LPXB reverse 473
ggcgcttcaatacccacgaacacatcagg

SEQ ID NO: 484 - DNA, 29 bp, Artificial
  primer LPXB reverse 474
ggcgcttcaatacccacgaacacatccgg

SEQ ID NO: 485 - DNA, 29 bp, Artificial
  primer LPXB reverse 475
ggcgcttcaatacccacgaacacatctgg

SEQ ID NO: 486 - DNA, 29 bp, Artificial
  primer LPXB reverse 476
ggcgcttcaatacccacgaacacatcggg

SEQ ID NO: 487 - DNA, 29 bp, Artificial
  primer LPXB reverse 477
ggcgcttcaatacccacgaagacatcagg

SEQ ID NO: 488 - DNA, 29 bp, Artificial
  primer LPXB reverse 478
ggcgcttcaatacccacgaagacatccgg

SEQ ID NO: 489 - DNA, 29 bp, Artificial
  primer LPXB reverse 479
ggcgcttcaatacccacgaagacatcggg

SEQ ID NO: 490 - DNA, 29 bp, Artificial
  primer LPXB reverse 480
ggcgcttcaatacccacgaagacatctgg

SEQ ID NO: 491 - DNA, 29 bp, Artificial
  primer LPXB reverse 481
ggcgcttcaataccgacaaaaacatcagg

SEQ ID NO: 492 - DNA, 29 bp, Artificial
  primer LPXB reverse 482
ggcgcttcaataccgacaaaaacatccgg

SEQ ID NO: 493 - DNA, 29 bp, Artificial
  primer LPXB reverse 483
ggcgcttcaataccgacaaaaacatcggg

SEQ ID NO: 494 - DNA, 29 bp, Artificial
  primer LPXB reverse 484
ggcgcttcaataccgacaaaaacatctgg

SEQ ID NO: 495 - DNA, 29 bp, Artificial
  primer LPXB reverse 485
ggcgcttcaataccgacaaacacatcagg

SEQ ID NO: 496 - DNA, 29 bp, Artificial
  primer LPXB reverse 486
ggcgcttcaataccgacaaacacatccgg

SEQ ID NO: 497 - DNA, 29 bp, Artificial
  primer LPXB reverse 487
ggcgcttcaataccgacaaacacatctgg

SEQ ID NO: 498 - DNA, 29 bp, Artificial
  primer LPXB reverse 488
ggcgcttcaataccgacaaacacatcggg

SEQ ID NO: 499 - DNA, 29 bp, Artificial
  primer LPXB reverse 489
ggcgcttcaataccgacaaagacatcagg

SEQ ID NO: 500 - DNA, 29 bp, Artificial
  primer LPXB reverse 490
ggcgcttcaataccgacaaagacatccgg

SEQ ID NO: 501 - DNA, 29 bp, Artificial
  primer LPXB reverse 491
ggcgcttcaataccgacaaagacatcggg

SEQ ID NO: 502 - DNA, 29 bp, Artificial
  primer LPXB reverse 492
ggcgcttcaataccgacaaagacatctgg

SEQ ID NO: 503 - DNA, 29 bp, Artificial
  primer LPXB reverse 493
ggcgcttcaataccgacgaaaacatcagg

SEQ ID NO: 504 - DNA, 29 bp, Artificial
  primer LPXB reverse 494
ggcgcttcaataccgacgaaaacatccgg

SEQ ID NO: 505 - DNA, 29 bp, Artificial
  primer LPXB reverse 495
ggcgcttcaataccgacgaaaacatcggg

SEQ ID NO: 506 - DNA, 29 bp, Artificial
  primer LPXB reverse 496
ggcgcttcaataccgacgaaaacatctgg

SEQ ID NO: 507 - DNA, 29 bp, Artificial
  primer LPXB reverse 497
ggcgcttcaataccgacgaacacatcagg

SEQ ID NO: 508 - DNA, 29 bp, Artificial
  primer LPXB reverse 498
ggcgcttcaataccgacgaacacatccgg

SEQ ID NO: 509 - DNA, 29 bp, Artificial
  primer LPXB reverse 499
ggcgcttcaataccgacgaacacatctgg

SEQ ID NO: 510 - DNA, 29 bp, Artificial
  primer LPXB reverse 500
ggcgcttcaataccgacgaacacatcggg

SEQ ID NO: 511 - DNA, 29 bp, Artificial
  primer LPXB reverse 501
ggcgcttcaataccgacgaagacatcagg

SEQ ID NO: 512 - DNA, 29 bp, Artificial
  primer LPXB reverse 502
ggcgcttcaataccgacgaagacatccgg

SEQ ID NO: 513 - DNA, 29 bp, Artificial
  primer LPXB reverse 503
ggcgcttcaataccgacgaagacatcggg

SEQ ID NO: 514 - DNA, 29 bp, Artificial
  primer LPXB reverse 504
ggcgcttcaataccgacgaagacatctgg

SEQ ID NO: 515 - DNA, 29 bp, Artificial
  primer LPXB reverse 505
ggcgcttcaatcccaacaaaaacatcagg

SEQ ID NO: 516 - DNA, 29 bp, Artificial
  primer LPXB reverse 506
ggcgcttcaatcccaacaaaaacatccgg

SEQ ID NO: 517 - DNA, 29 bp, Artificial
  primer LPXB reverse 507
ggcgcttcaatcccaacaaaaacatcggg

SEQ ID NO: 518 - DNA, 29 bp, Artificial
  primer LPXB reverse 508
ggcgcttcaatcccaacaaaaacatctgg

SEQ ID NO: 519 - DNA, 29 bp, Artificial
  primer LPXB reverse 509
ggcgcttcaatcccaacaaacacatcagg

SEQ ID NO: 520 - DNA, 29 bp, Artificial
  primer LPXB reverse 510
ggcgcttcaatcccaacaaacacatccgg

SEQ ID NO: 521 - DNA, 29 bp, Artificial
  primer LPXB reverse 511
ggcgcttcaatcccaacaaacacatctgg

SEQ ID NO: 522 - DNA, 29 bp, Artificial
  primer LPXB reverse 512
ggcgcttcaatcccaacaaacacatcggg

SEQ ID NO: 523 - DNA, 29 bp, Artificial
  primer LPXB reverse 513
ggcgcttcaatcccaacaaagacatcagg

SEQ ID NO: 524 - DNA, 29 bp, Artificial
  primer LPXB reverse 514
ggcgcttcaatcccaacaaagacatccgg

SEQ ID NO: 525 - DNA, 29 bp, Artificial
  primer LPXB reverse 515
ggcgcttcaatcccaacaaagacatcggg

SEQ ID NO: 526 - DNA, 29 bp, Artificial
  primer LPXB reverse 516
ggcgcttcaatcccaacaaagacatctgg

SEQ ID NO: 527 - DNA, 29 bp, Artificial
  primer LPXB reverse 517
ggcgcttcaatcccaacgaaaacatcagg

SEQ ID NO: 528 - DNA, 29 bp, Artificial
  primer LPXB reverse 518
ggcgcttcaatcccaacgaaaacatccgg

SEQ ID NO: 529 - DNA, 29 bp, Artificial
  primer LPXB reverse 519
ggcgcttcaatcccaacgaaaacatcggg

SEQ ID NO: 530 - DNA, 29 bp, Artificial
  primer LPXB reverse 520
ggcgcttcaatcccaacgaaaacatctgg

SEQ ID NO: 531 - DNA, 29 bp, Artificial
  primer LPXB reverse 521
ggcgcttcaatcccaacgaacacatcagg

SEQ ID NO: 532 - DNA, 29 bp, Artificial
  primer LPXB reverse 522
ggcgcttcaatcccaacgaacacatccgg

SEQ ID NO: 533 - DNA, 29 bp, Artificial
  primer LPXB reverse 523
ggcgcttcaatcccaacgaacacatctgg

SEQ ID NO: 534 - DNA, 29 bp, Artificial
  primer LPXB reverse 524
ggcgcttcaatcccaacgaacacatcggg

SEQ ID NO: 535 - DNA, 29 bp, Artificial
  primer LPXB reverse 525
ggcgcttcaatcccaacgaagacatcagg

SEQ ID NO: 536 - DNA, 29 bp, Artificial
  primer LPXB reverse 526
ggcgcttcaatcccaacgaagacatccgg

SEQ ID NO: 537 - DNA, 29 bp, Artificial
  primer LPXB reverse 527
ggcgcttcaatcccaacgaagacatcggg

SEQ ID NO: 538 - DNA, 29 bp, Artificial
  primer LPXB reverse 528
ggcgcttcaatcccaacgaagacatctgg

SEQ ID NO: 539 - DNA, 29 bp, Artificial
  primer LPXB reverse 529
ggcgcttcaatccccacaaaaacatcagg

SEQ ID NO: 540 - DNA, 29 bp, Artificial
  primer LPXB reverse 530
ggcgcttcaatccccacaaaaacatccgg

SEQ ID NO: 541 - DNA, 29 bp, Artificial
  primer LPXB reverse 531
ggcgcttcaatccccacaaaaacatcggg

SEQ ID NO: 542 - DNA, 29 bp, Artificial
  primer LPXB reverse 532
ggcgcttcaatccccacaaaaacatctgg

SEQ ID NO: 543 - DNA, 29 bp, Artificial
  primer LPXB reverse 533
ggcgcttcaatccccacaaacacatcagg

SEQ ID NO: 544 - DNA, 29 bp, Artificial
  primer LPXB reverse 534
ggcgcttcaatccccacaaacacatccgg

SEQ ID NO: 545 - DNA, 29 bp, Artificial
  primer LPXB reverse 535
ggcgcttcaatccccacaaacacatctgg

SEQ ID NO: 546 - DNA, 29 bp, Artificial
  primer LPXB reverse 536
ggcgcttcaatccccacaaacacatcggg

SEQ ID NO: 547 - DNA, 29 bp, Artificial
  primer LPXB reverse 537
ggcgcttcaatccccacaaagacatcagg

SEQ ID NO: 548 - DNA, 29 bp, Artificial
  primer LPXB reverse 538
ggcgcttcaatccccacaaagacatccgg

SEQ ID NO: 549 - DNA, 29 bp, Artificial
  primer LPXB reverse 539
ggcgcttcaatccccacaaagacatcggg

SEQ ID NO: 550 - DNA, 29 bp, Artificial
  primer LPXB reverse 540
ggcgcttcaatccccacaaagacatctgg

SEQ ID NO: 551 - DNA, 29 bp, Artificial
  primer LPXB reverse 541
ggcgcttcaatccccacgaaaacatcagg

SEQ ID NO: 552 - DNA, 29 bp, Artificial
  primer LPXB reverse 542
ggcgcttcaatccccacgaaaacatccgg

SEQ ID NO: 553 - DNA, 29 bp, Artificial
  primer LPXB reverse 543
ggcgcttcaatccccacgaaaacatcggg

SEQ ID NO: 554 - DNA, 29 bp, Artificial
  primer LPXB reverse 544
ggcgcttcaatccccacgaaaacatctgg

SEQ ID NO: 555 - DNA, 29 bp, Artificial
  primer LPXB reverse 545
ggcgcttcaatccccacgaacacatcagg

SEQ ID NO: 556 - DNA, 29 bp, Artificial
  primer LPXB reverse 546
ggcgcttcaatccccacgaacacatccgg

SEQ ID NO: 557 - DNA, 29 bp, Artificial
  primer LPXB reverse 547
ggcgcttcaatccccacgaacacatctgg

SEQ ID NO: 558 - DNA, 29 bp, Artificial
  primer LPXB reverse 548
ggcgcttcaatccccacgaacacatcggg

SEQ ID NO: 559 - DNA, 29 bp, Artificial
  primer LPXB reverse 549
ggcgcttcaatccccacgaagacatcagg

SEQ ID NO: 560 - DNA, 29 bp, Artificial
  primer LPXB reverse 550
ggcgcttcaatccccacgaagacatccgg

SEQ ID NO: 561 - DNA, 29 bp, Artificial
  primer LPXB reverse 551
ggcgcttcaatccccacgaagacatcggg

SEQ ID NO: 562 - DNA, 29 bp, Artificial
  primer LPXB reverse 552
ggcgcttcaatccccacgaagacatctgg

SEQ ID NO: 563 - DNA, 29 bp, Artificial
  primer LPXB reverse 553
ggcgcttcaatcccgacaaaaacatcagg

SEQ ID NO: 564 - DNA, 29 bp, Artificial
  primer LPXB reverse 554
ggcgcttcaatcccgacaaaaacatccgg

SEQ ID NO: 565 - DNA, 29 bp, Artificial
  primer LPXB reverse 555
ggcgcttcaatcccgacaaaaacatcggg

SEQ ID NO: 566 - DNA, 29 bp, Artificial
  primer LPXB reverse 556
ggcgcttcaatcccgacaaaaacatctgg

SEQ ID NO: 567 - DNA, 29 bp, Artificial
  primer LPXB reverse 557
ggcgcttcaatcccgacaaacacatcagg

SEQ ID NO: 568 - DNA, 29 bp, Artificial
  primer LPXB reverse 558
ggcgcttcaatcccgacaaacacatccgg

SEQ ID NO: 569 - DNA, 29 bp, Artificial
  primer LPXB reverse 559
ggcgcttcaatcccgacaaacacatctgg

SEQ ID NO: 570 - DNA, 29 bp, Artificial
  primer LPXB reverse 560
ggcgcttcaatcccgacaaacacatcggg

SEQ ID NO: 571 - DNA, 29 bp, Artificial
  primer LPXB reverse 561
ggcgcttcaatcccgacaaagacatcagg

SEQ ID NO: 572 - DNA, 29 bp, Artificial
  primer LPXB reverse 562
ggcgcttcaatcccgacaaagacatccgg

SEQ ID NO: 573 - DNA, 29 bp, Artificial
  primer LPXB reverse 563
ggcgcttcaatcccgacaaagacatcggg

SEQ ID NO: 574 - DNA, 29 bp, Artificial
  primer LPXB reverse 564
ggcgcttcaatcccgacaaagacatctgg

SEQ ID NO: 575 - DNA, 29 bp, Artificial
  primer LPXB reverse 565
ggcgcttcaatcccgacgaaaacatcagg

SEQ ID NO: 576 - DNA, 29 bp, Artificial
  primer LPXB reverse 566
ggcgcttcaatcccgacgaaaacatccgg

SEQ ID NO: 577 - DNA, 29 bp, Artificial
  primer LPXB reverse 567
ggcgcttcaatcccgacgaaaacatcggg

SEQ ID NO: 578 - DNA, 29 bp, Artificial
  primer LPXB reverse 568
ggcgcttcaatcccgacgaaaacatctgg

SEQ ID NO: 579 - DNA, 29 bp, Artificial
  primer LPXB reverse 569
ggcgcttcaatcccgacgaacacatcagg

SEQ ID NO: 580 - DNA, 29 bp, Artificial
  primer LPXB reverse 570
ggcgcttcaatcccgacgaacacatccgg

SEQ ID NO: 581 - DNA, 29 bp, Artificial
  primer LPXB reverse 571
ggcgcttcaatcccgacgaacacatctgg

SEQ ID NO: 582 - DNA, 29 bp, Artificial
  primer LPXB reverse 572
ggcgcttcaatcccgacgaacacatcggg

SEQ ID NO: 583 - DNA, 29 bp, Artificial
  primer LPXB reverse 573
ggcgcttcaatcccgacgaagacatcagg

SEQ ID NO: 584 - DNA, 29 bp, Artificial
  primer LPXB reverse 574
ggcgcttcaatcccgacgaagacatccgg

SEQ ID NO: 585 - DNA, 29 bp, Artificial
  primer LPXB reverse 575
ggcgcttcaatcccgacgaagacatcggg

SEQ ID NO: 586 - DNA, 29 bp, Artificial
  primer LPXB reverse 576
ggcgcttcaatcccgacgaagacatctgg

SEQ ID NO: 587 - DNA, 29 bp, Artificial
  primer LPXB reverse 577
ggcgcttcaattccaacaaaaacatcagg

SEQ ID NO: 588 - DNA, 29 bp, Artificial
  primer LPXB reverse 578
ggcgcttcaattccaacaaaaacatccgg

SEQ ID NO: 589 - DNA, 29 bp, Artificial
  primer LPXB reverse 579
ggcgcttcaattccaacaaaaacatcggg

SEQ ID NO: 590 - DNA, 29 bp, Artificial
  primer LPXB reverse 580
ggcgcttcaattccaacaaaaacatctgg

SEQ ID NO: 591 - DNA, 29 bp, Artificial
  primer LPXB reverse 581
ggcgcttcaattccaacaaacacatcagg

SEQ ID NO: 592 - DNA, 29 bp, Artificial
  primer LPXB reverse 582
ggcgcttcaattccaacaaacacatccgg

SEQ ID NO: 593 - DNA, 29 bp, Artificial
  primer LPXB reverse 583
ggcgcttcaattccaacaaacacatctgg

SEQ ID NO: 594 - DNA, 29 bp, Artificial
  primer LPXB reverse 584
ggcgcttcaattccaacaaacacatcggg

SEQ ID NO: 595 - DNA, 29 bp, Artificial
  primer LPXB reverse 585
ggcgcttcaattccaacaaagacatcagg

SEQ ID NO: 596 - DNA, 29 bp, Artificial
  primer LPXB reverse 586
ggcgcttcaattccaacaaagacatccgg

SEQ ID NO: 597 - DNA, 29 bp, Artificial
  primer LPXB reverse 587
ggcgcttcaattccaacaaagacatcggg

SEQ ID NO: 598 - DNA, 29 bp, Artificial
  primer LPXB reverse 588
ggcgcttcaattccaacaaagacatctgg

SEQ ID NO: 599 - DNA, 29 bp, Artificial
  primer LPXB reverse 589
ggcgcttcaattccaacgaaaacatcagg

SEQ ID NO: 600 - DNA, 29 bp, Artificial
  primer LPXB reverse 590
ggcgcttcaattccaacgaaaacatccgg

SEQ ID NO: 601 - DNA, 29 bp, Artificial
  primer LPXB reverse 591
ggcgcttcaattccaacgaaaacatcggg

SEQ ID NO: 602 - DNA, 29 bp, Artificial
  primer LPXB reverse 592
ggcgcttcaattccaacgaaaacatctgg

SEQ ID NO: 603 - DNA, 29 bp, Artificial
  primer LPXB reverse 593
ggcgcttcaattccaacgaacacatcagg

SEQ ID NO: 604 - DNA, 29 bp, Artificial
  primer LPXB reverse 594
ggcgcttcaattccaacgaacacatccgg

SEQ ID NO: 605 - DNA, 29 bp, Artificial
  primer LPXB reverse 595
ggcgcttcaattccaacgaacacatctgg

SEQ ID NO: 606 - DNA, 29 bp, Artificial
  primer LPXB reverse 596
ggcgcttcaattccaacgaacacatcggg

SEQ ID NO: 607 - DNA, 29 bp, Artificial
  primer LPXB reverse 597
ggcgcttcaattccaacgaagacatcagg

SEQ ID NO: 608 - DNA, 29 bp, Artificial
  primer LPXB reverse 598
ggcgcttcaattccaacgaagacatccgg

SEQ ID NO: 609 - DNA, 29 bp, Artificial
  primer LPXB reverse 599
ggcgcttcaattccaacgaagacatcggg

SEQ ID NO: 610 - DNA, 29 bp, Artificial
  primer LPXB reverse 600
ggcgcttcaattccaacgaagacatctgg

SEQ ID NO: 611 - DNA, 29 bp, Artificial
  primer LPXB reverse 601
ggcgcttcaattcccacaaaaacatcagg

SEQ ID NO: 612 - DNA, 29 bp, Artificial
  primer LPXB reverse 602
ggcgcttcaattcccacaaaaacatccgg

SEQ ID NO: 613 - DNA, 29 bp, Artificial
  primer LPXB reverse 603
ggcgcttcaattcccacaaaaacatcggg

SEQ ID NO: 614 - DNA, 29 bp, Artificial
  primer LPXB reverse 604
ggcgcttcaattcccacaaaaacatctgg

SEQ ID NO: 615 - DNA, 29 bp, Artificial
  primer LPXB reverse 605
ggcgcttcaattcccacaaacacatcagg

SEQ ID NO: 616 - DNA, 29 bp, Artificial
  primer LPXB reverse 606
ggcgcttcaattcccacaaacacatccgg

SEQ ID NO: 617 - DNA, 29 bp, Artificial
  primer LPXB reverse 607
ggcgcttcaattcccacaaacacatctgg

SEQ ID NO: 618 - DNA, 29 bp, Artificial
  primer LPXB reverse 608
ggcgcttcaattcccacaaacacatcggg

SEQ ID NO: 619 - DNA, 29 bp, Artificial
  primer LPXB reverse 609
ggcgcttcaattcccacaaagacatcagg

SEQ ID NO: 620 - DNA, 29 bp, Artificial
  primer LPXB reverse 610
ggcgcttcaattcccacaaagacatccgg

SEQ ID NO: 621 - DNA, 29 bp, Artificial
  primer LPXB reverse 611
ggcgcttcaattcccacaaagacatcggg

SEQ ID NO: 622 - DNA, 29 bp, Artificial
  primer LPXB reverse 612
ggcgcttcaattcccacaaagacatctgg

SEQ ID NO: 623 - DNA, 29 bp, Artificial
  primer LPXB reverse 613
ggcgcttcaattcccacgaaaacatcagg

SEQ ID NO: 624 - DNA, 29 bp, Artificial
  primer LPXB reverse 614
ggcgcttcaattcccacgaaaacatccgg

SEQ ID NO: 625 - DNA, 29 bp, Artificial
  primer LPXB reverse 615
ggcgcttcaattcccacgaaaacatcggg

SEQ ID NO: 626 - DNA, 29 bp, Artificial
  primer LPXB reverse 616
ggcgcttcaattcccacgaaaacatctgg

SEQ ID NO: 627 - DNA, 29 bp, Artificial
  primer LPXB reverse 617
ggcgcttcaattcccacgaacacatcagg

SEQ ID NO: 628 - DNA, 29 bp, Artificial
  primer LPXB reverse 618
ggcgcttcaattcccacgaacacatccgg

SEQ ID NO: 629 - DNA, 29 bp, Artificial
  primer LPXB reverse 619
ggcgcttcaattcccacgaacacatctgg

SEQ ID NO: 630 - DNA, 29 bp, Artificial
  primer LPXB reverse 620
ggcgcttcaattcccacgaacacatcggg

SEQ ID NO: 631 - DNA, 29 bp, Artificial
  primer LPXB reverse 621
ggcgcttcaattcccacgaagacatcagg

SEQ ID NO: 632 - DNA, 29 bp, Artificial
  primer LPXB reverse 622
ggcgcttcaattcccacgaagacatccgg

SEQ ID NO: 633 - DNA, 29 bp, Artificial
  primer LPXB reverse 623
ggcgcttcaattcccacgaagacatcggg

SEQ ID NO: 634 - DNA, 29 bp, Artificial
  primer LPXB reverse 624
ggcgcttcaattcccacgaagacatctgg

SEQ ID NO: 635 - DNA, 29 bp, Artificial
  primer LPXB reverse 625
ggcgcttcaattccgacaaaaacatcagg

SEQ ID NO: 636 - DNA, 29 bp, Artificial
  primer LPXB reverse 626
ggcgcttcaattccgacaaaaacatccgg

SEQ ID NO: 637 - DNA, 29 bp, Artificial
  primer LPXB reverse 627
ggcgcttcaattccgacaaaaacatcggg

SEQ ID NO: 638 - DNA, 29 bp, Artificial
  primer LPXB reverse 628
ggcgcttcaattccgacaaaaacatctgg

SEQ ID NO: 639 - DNA, 29 bp, Artificial
  primer LPXB reverse 629
ggcgcttcaattccgacaaacacatcagg

SEQ ID NO: 640 - DNA, 29 bp, Artificial
  primer LPXB reverse 630
ggcgcttcaattccgacaaacacatccgg

SEQ ID NO: 641 - DNA, 29 bp, Artificial
  primer LPXB reverse 631
ggcgcttcaattccgacaaacacatctgg

SEQ ID NO: 642 - DNA, 29 bp, Artificial
  primer LPXB reverse 632
ggcgcttcaattccgacaaacacatcggg

SEQ ID NO: 643 - DNA, 29 bp, Artificial
  primer LPXB reverse 633
ggcgcttcaattccgacaaagacatcagg

SEQ ID NO: 644 - DNA, 29 bp, Artificial
  primer LPXB reverse 634
ggcgcttcaattccgacaaagacatccgg

SEQ ID NO: 645 - DNA, 29 bp, Artificial
  primer LPXB reverse 635
ggcgcttcaattccgacaaagacatcggg

SEQ ID NO: 646 - DNA, 29 bp, Artificial
  primer LPXB reverse 636
ggcgcttcaattccgacaaagacatctgg

SEQ ID NO: 647 - DNA, 29 bp, Artificial
  primer LPXB reverse 637
ggcgcttcaattccgacgaaaacatcagg

SEQ ID NO: 648 - DNA, 29 bp, Artificial
  primer LPXB reverse 638
ggcgcttcaattccgacgaaaacatccgg

SEQ ID NO: 649 - DNA, 29 bp, Artificial
  primer LPXB reverse 639
ggcgcttcaattccgacgaaaacatcggg

SEQ ID NO: 650 - DNA, 29 bp, Artificial
  primer LPXB reverse 640
ggcgcttcaattccgacgaaaacatctgg

SEQ ID NO: 651 - DNA, 29 bp, Artificial
  primer LPXB reverse 641
ggcgcttcaattccgacgaacacatcagg

SEQ ID NO: 652 - DNA, 29 bp, Artificial
  primer LPXB reverse 642
ggcgcttcaattccgacgaacacatccgg

SEQ ID NO: 653 - DNA, 29 bp, Artificial
  primer LPXB reverse 643
ggcgcttcaattccgacgaacacatctgg

SEQ ID NO: 654 - DNA, 29 bp, Artificial
  primer LPXB reverse 644
ggcgcttcaattccgacgaacacatcggg

SEQ ID NO: 655 - DNA, 29 bp, Artificial
  primer LPXB reverse 645
ggcgcttcaattccgacgaagacatcagg

SEQ ID NO: 656 - DNA, 29 bp, Artificial
  primer LPXB reverse 646
ggcgcttcaattccgacgaagacatccgg

SEQ ID NO: 657 - DNA, 29 bp, Artificial
  primer LPXB reverse 647
ggcgcttcaattccgacgaagacatcggg

SEQ ID NO: 658 - DNA, 29 bp, Artificial
  primer LPXB reverse 648
ggcgcttcaattccgacgaagacatctgg

SEQ ID NO: 659 - DNA, 29 bp, Artificial
  primer LPXB reverse 649
ggcgcttcgataccaacaaaaacatcagg

SEQ ID NO: 660 - DNA, 29 bp, Artificial
  primer LPXB reverse 650
ggcgcttcgataccaacaaaaacatccgg

SEQ ID NO: 661 - DNA, 29 bp, Artificial
  primer LPXB reverse 651
ggcgcttcgataccaacaaaaacatcggg

SEQ ID NO: 662 - DNA, 29 bp, Artificial
  primer LPXB reverse 652
ggcgcttcgataccaacaaaaacatctgg

SEQ ID NO: 663 - DNA, 29 bp, Artificial
  primer LPXB reverse 653
ggcgcttcgataccaacaaacacatcagg

SEQ ID NO: 664 - DNA, 29 bp, Artificial
  primer LPXB reverse 654
ggcgcttcgataccaacaaacacatccgg

SEQ ID NO: 665 - DNA, 29 bp, Artificial
  primer LPXB reverse 655
ggcgcttcgataccaacaaacacatctgg

SEQ ID NO: 666 - DNA, 29 bp, Artificial
  primer LPXB reverse 656
ggcgcttcgataccaacaaacacatcggg

SEQ ID NO: 667 - DNA, 29 bp, Artificial
  primer LPXB reverse 657
ggcgcttcgataccaacaaagacatcagg

SEQ ID NO: 668 - DNA, 29 bp, Artificial
  primer LPXB reverse 658
ggcgcttcgataccaacaaagacatccgg

SEQ ID NO: 669 - DNA, 29 bp, Artificial
  primer LPXB reverse 659
ggcgcttcgataccaacaaagacatcggg

SEQ ID NO: 670 - DNA, 29 bp, Artificial
  primer LPXB reverse 660
ggcgcttcgataccaacaaagacatctgg

SEQ ID NO: 671 - DNA, 29 bp, Artificial
  primer LPXB reverse 661
ggcgcttcgataccaacgaaaacatcagg

SEQ ID NO: 672 - DNA, 29 bp, Artificial
  primer LPXB reverse 662
ggcgcttcgataccaacgaaaacatccgg

SEQ ID NO: 673 - DNA, 29 bp, Artificial
  primer LPXB reverse 663
ggcgcttcgataccaacgaaaacatcggg

SEQ ID NO: 674 - DNA, 29 bp, Artificial
  primer LPXB reverse 664
ggcgcttcgataccaacgaaaacatctgg

SEQ ID NO: 675 - DNA, 29 bp, Artificial
  primer LPXB reverse 665
ggcgcttcgataccaacgaacacatcagg

SEQ ID NO: 676 - DNA, 29 bp, Artificial
  primer LPXB reverse 666
ggcgcttcgataccaacgaacacatccgg

SEQ ID NO: 677 - DNA, 29 bp, Artificial
  primer LPXB reverse 667
ggcgcttcgataccaacgaacacatctgg

SEQ ID NO: 678 - DNA, 29 bp, Artificial
  primer LPXB reverse 668
ggcgcttcgataccaacgaacacatcggg

SEQ ID NO: 679 - DNA, 29 bp, Artificial
  primer LPXB reverse 669
ggcgcttcgataccaacgaagacatcagg

SEQ ID NO: 680 - DNA, 29 bp, Artificial
  primer LPXB reverse 670
ggcgcttcgataccaacgaagacatccgg

SEQ ID NO: 681 - DNA, 29 bp, Artificial
  primer LPXB reverse 671
ggcgcttcgataccaacgaagacatcggg

SEQ ID NO: 682 - DNA, 29 bp, Artificial
  primer LPXB reverse 672
ggcgcttcgataccaacgaagacatctgg

SEQ ID NO: 683 - DNA, 29 bp, Artificial
  primer LPXB reverse 673
ggcgcttcgatacccacaaaaacatcagg

SEQ ID NO: 684 - DNA, 29 bp, Artificial
  primer LPXB reverse 674
ggcgcttcgatacccacaaaaacatccgg

SEQ ID NO: 685 - DNA, 29 bp, Artificial
  primer LPXB reverse 675
ggcgcttcgatacccacaaaaacatcggg

SEQ ID NO: 686 - DNA, 29 bp, Artificial
  primer LPXB reverse 676
ggcgcttcgatacccacaaaaacatctgg

SEQ ID NO: 687 - DNA, 29 bp, Artificial
  primer LPXB reverse 677
ggcgcttcgatacccacaaacacatcagg

SEQ ID NO: 688 - DNA, 29 bp, Artificial
  primer LPXB reverse 678
ggcgcttcgatacccacaaacacatccgg

SEQ ID NO: 689 - DNA, 29 bp, Artificial
  primer LPXB reverse 679
ggcgcttcgatacccacaaacacatctgg

SEQ ID NO: 690 - DNA, 29 bp, Artificial
  primer LPXB reverse 680
ggcgcttcgatacccacaaacacatcggg

SEQ ID NO: 691 - DNA, 29 bp, Artificial
  primer LPXB reverse 681
ggcgcttcgatacccacaaagacatcagg

SEQ ID NO: 692 - DNA, 29 bp, Artificial
  primer LPXB reverse 682
ggcgcttcgatacccacaaagacatccgg

SEQ ID NO: 693 - DNA, 29 bp, Artificial
  primer LPXB reverse 683
ggcgcttcgatacccacaaagacatcggg

SEQ ID NO: 694 - DNA, 29 bp, Artificial
  primer LPXB reverse 684
ggcgcttcgatacccacaaagacatctgg

SEQ ID NO: 695 - DNA, 29 bp, Artificial
  primer LPXB reverse 685
ggcgcttcgatacccacgaaaacatcagg

SEQ ID NO: 696 - DNA, 29 bp, Artificial
  primer LPXB reverse 686
ggcgcttcgatacccacgaaaacatccgg

SEQ ID NO: 697 - DNA, 29 bp, Artificial
  primer LPXB reverse 687
ggcgcttcgatacccacgaaaacatcggg

SEQ ID NO: 698 - DNA, 29 bp, Artificial
  primer LPXB reverse 688
ggcgcttcgatacccacgaaaacatctgg

SEQ ID NO: 699 - DNA, 29 bp, Artificial
  primer LPXB reverse 689
ggcgcttcgatacccacgaacacatcagg

SEQ ID NO: 700 - DNA, 29 bp, Artificial
  primer LPXB reverse 690
ggcgcttcgatacccacgaacacatccgg

SEQ ID NO: 701 - DNA, 29 bp, Artificial
  primer LPXB reverse 691
ggcgcttcgatacccacgaacacatctgg

SEQ ID NO: 702 - DNA, 29 bp, Artificial
  primer LPXB reverse 692
ggcgcttcgatacccacgaacacatcggg

SEQ ID NO: 703 - DNA, 29 bp, Artificial
  primer LPXB reverse 693
ggcgcttcgatacccacgaagacatcagg

SEQ ID NO: 704 - DNA, 29 bp, Artificial
  primer LPXB reverse 694
ggcgcttcgatacccacgaagacatccgg

SEQ ID NO: 705 - DNA, 29 bp, Artificial
  primer LPXB reverse 695
ggcgcttcgatacccacgaagacatcggg

SEQ ID NO: 706 - DNA, 29 bp, Artificial
  primer LPXB reverse 696
ggcgcttcgatacccacgaagacatctgg

SEQ ID NO: 707 - DNA, 29 bp, Artificial
  primer LPXB reverse 697
ggcgcttcgataccgacaaaaacatcagg

SEQ ID NO: 708 - DNA, 29 bp, Artificial
  primer LPXB reverse 698
ggcgcttcgataccgacaaaaacatccgg

SEQ ID NO: 709 - DNA, 29 bp, Artificial
  primer LPXB reverse 699
ggcgcttcgataccgacaaaaacatcggg

SEQ ID NO: 710 - DNA, 29 bp, Artificial
  primer LPXB reverse 700
ggcgcttcgataccgacaaaaacatctgg

SEQ ID NO: 711 - DNA, 29 bp, Artificial
  primer LPXB reverse 701
ggcgcttcgataccgacaaacacatcagg

SEQ ID NO: 712 - DNA, 29 bp, Artificial
  primer LPXB reverse 702
ggcgcttcgataccgacaaacacatccgg

SEQ ID NO: 713 - DNA, 29 bp, Artificial
  primer LPXB reverse 703
ggcgcttcgataccgacaaacacatctgg

SEQ ID NO: 714 - DNA, 29 bp, Artificial
  primer LPXB reverse 704
ggcgcttcgataccgacaaacacatcggg

SEQ ID NO: 715 - DNA, 29 bp, Artificial
  primer LPXB reverse 705
ggcgcttcgataccgacaaagacatcagg

SEQ ID NO: 716 - DNA, 29 bp, Artificial
  primer LPXB reverse 706
ggcgcttcgataccgacaaagacatccgg

SEQ ID NO: 717 - DNA, 29 bp, Artificial
  primer LPXB reverse 707
ggcgcttcgataccgacaaagacatcggg

SEQ ID NO: 718 - DNA, 29 bp, Artificial
  primer LPXB reverse 708
ggcgcttcgataccgacaaagacatctgg

SEQ ID NO: 719 - DNA, 29 bp, Artificial
  primer LPXB reverse 709
ggcgcttcgataccgacgaaaacatcagg

SEQ ID NO: 720 - DNA, 29 bp, Artificial
  primer LPXB reverse 710
ggcgcttcgataccgacgaaaacatccgg

SEQ ID NO: 721 - DNA, 29 bp, Artificial
  primer LPXB reverse 711
ggcgcttcgataccgacgaaaacatcggg

SEQ ID NO: 722 - DNA, 29 bp, Artificial
  primer LPXB reverse 712
ggcgcttcgataccgacgaaaacatctgg

SEQ ID NO: 723 - DNA, 29 bp, Artificial
  primer LPXB reverse 713
ggcgcttcgataccgacgaacacatcagg

SEQ ID NO: 724 - DNA, 29 bp, Artificial
  primer LPXB reverse 714
ggcgcttcgataccgacgaacacatccgg

SEQ ID NO: 725 - DNA, 29 bp, Artificial
  primer LPXB reverse 715
ggcgcttcgataccgacgaacacatctgg

SEQ ID NO: 726 - DNA, 29 bp, Artificial
  primer LPXB reverse 716
ggcgcttcgataccgacgaacacatcggg

SEQ ID NO: 727 - DNA, 29 bp, Artificial
  primer LPXB reverse 717
ggcgcttcgataccgacgaagacatcagg

SEQ ID NO: 728 - DNA, 29 bp, Artificial
  primer LPXB reverse 718
ggcgcttcgataccgacgaagacatccgg

SEQ ID NO: 729 - DNA, 29 bp, Artificial
  primer LPXB reverse 719
ggcgcttcgataccgacgaagacatcggg

SEQ ID NO: 730 - DNA, 29 bp, Artificial
  primer LPXB reverse 720
ggcgcttcgataccgacgaagacatctgg

SEQ ID NO: 731 - DNA, 29 bp, Artificial
  primer LPXB reverse 721
ggcgcttcgatcccaacaaaaacatcagg

SEQ ID NO: 732 - DNA, 29 bp, Artificial
  primer LPXB reverse 722
ggcgcttcgatcccaacaaaaacatccgg

SEQ ID NO: 733 - DNA, 29 bp, Artificial
  primer LPXB reverse 723
ggcgcttcgatcccaacaaaaacatcggg

SEQ ID NO: 734 - DNA, 29 bp, Artificial
  primer LPXB reverse 724
ggcgcttcgatcccaacaaaaacatctgg

SEQ ID NO: 735 - DNA, 29 bp, Artificial
  primer LPXB reverse 725
ggcgcttcgatcccaacaaacacatcagg

SEQ ID NO: 736 - DNA, 29 bp, Artificial
  primer LPXB reverse 726
ggcgcttcgatcccaacaaacacatccgg

SEQ ID NO: 737 - DNA, 29 bp, Artificial
  primer LPXB reverse 727
ggcgcttcgatcccaacaaacacatctgg

SEQ ID NO: 738 - DNA, 29 bp, Artificial
  primer LPXB reverse 728
ggcgcttcgatcccaacaaacacatcggg

SEQ ID NO: 739 - DNA, 29 bp, Artificial
  primer LPXB reverse 729
ggcgcttcgatcccaacaaagacatcagg

SEQ ID NO: 740 - DNA, 29 bp, Artificial
  primer LPXB reverse 730
ggcgcttcgatcccaacaaagacatccgg

SEQ ID NO: 741 - DNA, 29 bp, Artificial
  primer LPXB reverse 731
ggcgcttcgatcccaacaaagacatcggg

SEQ ID NO: 742 - DNA, 29 bp, Artificial
  primer LPXB reverse 732
ggcgcttcgatcccaacaaagacatctgg

SEQ ID NO: 743 - DNA, 29 bp, Artificial
  primer LPXB reverse 733
ggcgcttcgatcccaacgaaaacatcagg

SEQ ID NO: 744 - DNA, 29 bp, Artificial
  primer LPXB reverse 734
ggcgcttcgatcccaacgaaaacatccgg

SEQ ID NO: 745 - DNA, 29 bp, Artificial
  primer LPXB reverse 735
ggcgcttcgatcccaacgaaaacatcggg

SEQ ID NO: 746 - DNA, 29 bp, Artificial
  primer LPXB reverse 736
ggcgcttcgatcccaacgaaaacatctgg

SEQ ID NO: 747 - DNA, 29 bp, Artificial
  primer LPXB reverse 737
ggcgcttcgatcccaacgaacacatcagg

SEQ ID NO: 748 - DNA, 29 bp, Artificial
  primer LPXB reverse 738
ggcgcttcgatcccaacgaacacatccgg

SEQ ID NO: 749 - DNA, 29 bp, Artificial
  primer LPXB reverse 739
ggcgcttcgatcccaacgaacacatctgg

SEQ ID NO: 750 - DNA, 29 bp, Artificial
  primer LPXB reverse 740
ggcgcttcgatcccaacgaacacatcggg

SEQ ID NO: 751 - DNA, 29 bp, Artificial
  primer LPXB reverse 741
ggcgcttcgatcccaacgaagacatcagg

SEQ ID NO: 752 - DNA, 29 bp, Artificial
  primer LPXB reverse 742
ggcgcttcgatcccaacgaagacatccgg

SEQ ID NO: 753 - DNA, 29 bp, Artificial
  primer LPXB reverse 743
ggcgcttcgatcccaacgaagacatcggg

SEQ ID NO: 754 - DNA, 29 bp, Artificial
  primer LPXB reverse 744
ggcgcttcgatcccaacgaagacatctgg

SEQ ID NO: 755 - DNA, 29 bp, Artificial
  primer LPXB reverse 745
ggcgcttcgatccccacaaaaacatcagg

SEQ ID NO: 756 - DNA, 29 bp, Artificial
  primer LPXB reverse 746
ggcgcttcgatccccacaaaaacatccgg

SEQ ID NO: 757 - DNA, 29 bp, Artificial
  primer LPXB reverse 747
ggcgcttcgatccccacaaaaacatcggg

SEQ ID NO: 758 - DNA, 29 bp, Artificial
  primer LPXB reverse 748
ggcgcttcgatccccacaaaaacatctgg

SEQ ID NO: 759 - DNA, 29 bp, Artificial
  primer LPXB reverse 749
ggcgcttcgatccccacaaacacatcagg

SEQ ID NO: 760 - DNA, 29 bp, Artificial
  primer LPXB reverse 750
ggcgcttcgatccccacaaacacatccgg

SEQ ID NO: 761 - DNA, 29 bp, Artificial
  primer LPXB reverse 751
ggcgcttcgatccccacaaacacatctgg

SEQ ID NO: 762 - DNA, 29 bp, Artificial
  primer LPXB reverse 752
ggcgcttcgatccccacaaacacatcggg

SEQ ID NO: 763 - DNA, 29 bp, Artificial
  primer LPXB reverse 753
ggcgcttcgatccccacaaagacatcagg

SEQ ID NO: 764 - DNA, 29 bp, Artificial
  primer LPXB reverse 754
ggcgcttcgatccccacaaagacatccgg

SEQ ID NO: 765 - DNA, 29 bp, Artificial
  primer LPXB reverse 755
ggcgcttcgatccccacaaagacatcggg

SEQ ID NO: 766 - DNA, 29 bp, Artificial
  primer LPXB reverse 756
ggcgcttcgatccccacaaagacatctgg

SEQ ID NO: 767 - DNA, 29 bp, Artificial
  primer LPXB reverse 757
ggcgcttcgatccccacgaaaacatcagg

SEQ ID NO: 768 - DNA, 29 bp, Artificial
  primer LPXB reverse 758
ggcgcttcgatccccacgaaaacatccgg

SEQ ID NO: 769 - DNA, 29 bp, Artificial
  primer LPXB reverse 759
ggcgcttcgatccccacgaaaacatcggg

SEQ ID NO: 770 - DNA, 29 bp, Artificial
  primer LPXB reverse 760
ggcgcttcgatccccacgaaaacatctgg

SEQ ID NO: 771 - DNA, 29 bp, Artificial
  primer LPXB reverse 761
ggcgcttcgatccccacgaacacatcagg

SEQ ID NO: 772 - DNA, 29 bp, Artificial
  primer LPXB reverse 762
ggcgcttcgatccccacgaacacatccgg

SEQ ID NO: 773 - DNA, 29 bp, Artificial
  primer LPXB reverse 763
ggcgcttcgatccccacgaacacatctgg

SEQ ID NO: 774 - DNA, 29 bp, Artificial
  primer LPXB reverse 764
ggcgcttcgatccccacgaacacatcggg

SEQ ID NO: 775 - DNA, 29 bp, Artificial
  primer LPXB reverse 765
ggcgcttcgatccccacgaagacatcagg

SEQ ID NO: 776 - DNA, 29 bp, Artificial
  primer LPXB reverse 766
ggcgcttcgatccccacgaagacatccgg

SEQ ID NO: 777 - DNA, 29 bp, Artificial
  primer LPXB reverse 767
ggcgcttcgatccccacgaagacatcggg

SEQ ID NO: 778 - DNA, 29 bp, Artificial
  primer LPXB reverse 768
ggcgcttcgatccccacgaagacatctgg

SEQ ID NO: 779 - DNA, 29 bp, Artificial
  primer LPXB reverse 769
ggcgcttcgatcccgacaaaaacatcagg

SEQ ID NO: 780 - DNA, 29 bp, Artificial
  primer LPXB reverse 770
ggcgcttcgatcccgacaaaaacatccgg

SEQ ID NO: 781 - DNA, 29 bp, Artificial
  primer LPXB reverse 771
ggcgcttcgatcccgacaaaaacatcggg

SEQ ID NO: 782 - DNA, 29 bp, Artificial
  primer LPXB reverse 772
ggcgcttcgatcccgacaaaaacatctgg

SEQ ID NO: 783 - DNA, 29 bp, Artificial
  primer LPXB reverse 773
ggcgcttcgatcccgacaaacacatcagg

SEQ ID NO: 784 - DNA, 29 bp, Artificial
  primer LPXB reverse 774
ggcgcttcgatcccgacaaacacatccgg

SEQ ID NO: 785 - DNA, 29 bp, Artificial
  primer LPXB reverse 775
ggcgcttcgatcccgacaaacacatctgg

SEQ ID NO: 786 - DNA, 29 bp, Artificial
  primer LPXB reverse 776
ggcgcttcgatcccgacaaacacatcggg

SEQ ID NO: 787 - DNA, 29 bp, Artificial
  primer LPXB reverse 777
ggcgcttcgatcccgacaaagacatcagg

SEQ ID NO: 788 - DNA, 29 bp, Artificial
  primer LPXB reverse 778
ggcgcttcgatcccgacaaagacatccgg

SEQ ID NO: 789 - DNA, 29 bp, Artificial
  primer LPXB reverse 779
ggcgcttcgatcccgacaaagacatcggg

SEQ ID NO: 790 - DNA, 29 bp, Artificial
  primer LPXB reverse 780
ggcgcttcgatcccgacaaagacatctgg

SEQ ID NO: 791 - DNA, 29 bp, Artificial
  primer LPXB reverse 781
ggcgcttcgatcccgacgaaaacatcagg

SEQ ID NO: 792 - DNA, 29 bp, Artificial
  primer LPXB reverse 782
ggcgcttcgatcccgacgaaaacatccgg

SEQ ID NO: 793 - DNA, 29 bp, Artificial
  primer LPXB reverse 783
ggcgcttcgatcccgacgaaaacatcggg

SEQ ID NO: 794 - DNA, 29 bp, Artificial
  primer LPXB reverse 784
ggcgcttcgatcccgacgaaaacatctgg

SEQ ID NO: 795 - DNA, 29 bp, Artificial
  primer LPXB reverse 785
ggcgcttcgatcccgacgaacacatcagg

SEQ ID NO: 796 - DNA, 29 bp, Artificial
  primer LPXB reverse 786
ggcgcttcgatcccgacgaacacatccgg

SEQ ID NO: 797 - DNA, 29 bp, Artificial
  primer LPXB reverse 787
ggcgcttcgatcccgacgaacacatctgg

SEQ ID NO: 798 - DNA, 29 bp, Artificial
  primer LPXB reverse 788
ggcgcttcgatcccgacgaacacatcggg

SEQ ID NO: 799 - DNA, 29 bp, Artificial
  primer LPXB reverse 789
ggcgcttcgatcccgacgaagacatcagg

SEQ ID NO: 800 - DNA, 29 bp, Artificial
  primer LPXB reverse 790
ggcgcttcgatcccgacgaagacatccgg

SEQ ID NO: 801 - DNA, 29 bp, Artificial
  primer LPXB reverse 791
ggcgcttcgatcccgacgaagacatcggg

SEQ ID NO: 802 - DNA, 29 bp, Artificial
  primer LPXB reverse 792
ggcgcttcgatcccgacgaagacatctgg

SEQ ID NO: 803 - DNA, 29 bp, Artificial
  primer LPXB reverse 793
ggcgcttcgattccaacaaaaacatcagg

SEQ ID NO: 804 - DNA, 29 bp, Artificial
  primer LPXB reverse 794
ggcgcttcgattccaacaaaaacatccgg

SEQ ID NO: 805 - DNA, 29 bp, Artificial
  primer LPXB reverse 795
ggcgcttcgattccaacaaaaacatcggg

SEQ ID NO: 806 - DNA, 29 bp, Artificial
  primer LPXB reverse 796
ggcgcttcgattccaacaaaaacatctgg

SEQ ID NO: 807 - DNA, 29 bp, Artificial
  primer LPXB reverse 797
ggcgcttcgattccaacaaacacatcagg

SEQ ID NO: 808 - DNA, 29 bp, Artificial
  primer LPXB reverse 798
ggcgcttcgattccaacaaacacatccgg

SEQ ID NO: 809 - DNA, 29 bp, Artificial
  primer LPXB reverse 799
ggcgcttcgattccaacaaacacatctgg

SEQ ID NO: 810 - DNA, 29 bp, Artificial
  primer LPXB reverse 800
ggcgcttcgattccaacaaacacatcggg

SEQ ID NO: 811 - DNA, 29 bp, Artificial
  primer LPXB reverse 801
ggcgcttcgattccaacaaagacatcagg

SEQ ID NO: 812 - DNA, 29 bp, Artificial
  primer LPXB reverse 802
ggcgcttcgattccaacaaagacatccgg

SEQ ID NO: 813 - DNA, 29 bp, Artificial
  primer LPXB reverse 803
ggcgcttcgattccaacaaagacatcggg

SEQ ID NO: 814 - DNA, 29 bp, Artificial
  primer LPXB reverse 804
ggcgcttcgattccaacaaagacatctgg

SEQ ID NO: 815 - DNA, 29 bp, Artificial
  primer LPXB reverse 805
ggcgcttcgattccaacgaaaacatcagg

SEQ ID NO: 816 - DNA, 29 bp, Artificial
  primer LPXB reverse 806
ggcgcttcgattccaacgaaaacatccgg

SEQ ID NO: 817 - DNA, 29 bp, Artificial
  primer LPXB reverse 807
ggcgcttcgattccaacgaaaacatcggg

SEQ ID NO: 818 - DNA, 29 bp, Artificial
  primer LPXB reverse 808
ggcgcttcgattccaacgaaaacatctgg

SEQ ID NO: 819 - DNA, 29 bp, Artificial
  primer LPXB reverse 809
ggcgcttcgattccaacgaacacatcagg

SEQ ID NO: 820 - DNA, 29 bp, Artificial
  primer LPXB reverse 810
ggcgcttcgattccaacgaacacatccgg

SEQ ID NO: 821 - DNA, 29 bp, Artificial
  primer LPXB reverse 811
ggcgcttcgattccaacgaacacatctgg

SEQ ID NO: 822 - DNA, 29 bp, Artificial
  primer LPXB reverse 812
ggcgcttcgattccaacgaacacatcggg

SEQ ID NO: 823 - DNA, 29 bp, Artificial
  primer LPXB reverse 813
ggcgcttcgattccaacgaagacatcagg

SEQ ID NO: 824 - DNA, 29 bp, Artificial
  primer LPXB reverse 814
ggcgcttcgattccaacgaagacatccgg

SEQ ID NO: 825 - DNA, 29 bp, Artificial
  primer LPXB reverse 815
ggcgcttcgattccaacgaagacatcggg

SEQ ID NO: 826 - DNA, 29 bp, Artificial
  primer LPXB reverse 816
ggcgcttcgattccaacgaagacatctgg

SEQ ID NO: 827 - DNA, 29 bp, Artificial
  primer LPXB reverse 817
ggcgcttcgattcccacaaaaacatcagg

SEQ ID NO: 828 - DNA, 29 bp, Artificial
  primer LPXB reverse 818
ggcgcttcgattcccacaaaaacatccgg

SEQ ID NO: 829 - DNA, 29 bp, Artificial
  primer LPXB reverse 819
ggcgcttcgattcccacaaaaacatcggg

SEQ ID NO: 830 - DNA, 29 bp, Artificial
  primer LPXB reverse 820
ggcgcttcgattcccacaaaaacatctgg

SEQ ID NO: 831 - DNA, 29 bp, Artificial
  primer LPXB reverse 821
ggcgcttcgattcccacaaacacatcagg

SEQ ID NO: 832 - DNA, 29 bp, Artificial
  primer LPXB reverse 822
ggcgcttcgattcccacaaacacatccgg

SEQ ID NO: 833 - DNA, 29 bp, Artificial
  primer LPXB reverse 823
ggcgcttcgattcccacaaacacatctgg

SEQ ID NO: 834 - DNA, 29 bp, Artificial
  primer LPXB reverse 824
ggcgcttcgattcccacaaacacatcggg

SEQ ID NO: 835 - DNA, 29 bp, Artificial
  primer LPXB reverse 825
ggcgcttcgattcccacaaagacatcagg

SEQ ID NO: 836 - DNA, 29 bp, Artificial
  primer LPXB reverse 826
ggcgcttcgattcccacaaagacatccgg

SEQ ID NO: 837 - DNA, 29 bp, Artificial
  primer LPXB reverse 827
ggcgcttcgattcccacaaagacatcggg

SEQ ID NO: 838 - DNA, 29 bp, Artificial
  primer LPXB reverse 828
ggcgcttcgattcccacaaagacatctgg

SEQ ID NO: 839 - DNA, 29 bp, Artificial
  primer LPXB reverse 829
ggcgcttcgattcccacgaaaacatcagg

SEQ ID NO: 840 - DNA, 29 bp, Artificial
  primer LPXB reverse 830
ggcgcttcgattcccacgaaaacatccgg

SEQ ID NO: 841 - DNA, 29 bp, Artificial
  primer LPXB reverse 831
ggcgcttcgattcccacgaaaacatcggg

SEQ ID NO: 842 - DNA, 29 bp, Artificial
  primer LPXB reverse 832
ggcgcttcgattcccacgaaaacatctgg

SEQ ID NO: 843 - DNA, 29 bp, Artificial
  primer LPXB reverse 833
ggcgcttcgattcccacgaacacatcagg

SEQ ID NO: 844 - DNA, 29 bp, Artificial
  primer LPXB reverse 834
ggcgcttcgattcccacgaacacatccgg

SEQ ID NO: 845 - DNA, 29 bp, Artificial
  primer LPXB reverse 835
ggcgcttcgattcccacgaacacatctgg

SEQ ID NO: 846 - DNA, 29 bp, Artificial
  primer LPXB reverse 836
ggcgcttcgattcccacgaacacatcggg

SEQ ID NO: 847 - DNA, 29 bp, Artificial
  primer LPXB reverse 837
ggcgcttcgattcccacgaagacatcagg

SEQ ID NO: 848 - DNA, 29 bp, Artificial
  primer LPXB reverse 838
ggcgcttcgattcccacgaagacatccgg

SEQ ID NO: 849 - DNA, 29 bp, Artificial
  primer LPXB reverse 839
ggcgcttcgattcccacgaagacatcggg

SEQ ID NO: 850 - DNA, 29 bp, Artificial
  primer LPXB reverse 840
ggcgcttcgattcccacgaagacatctgg

SEQ ID NO: 851 - DNA, 29 bp, Artificial
  primer LPXB reverse 841
ggcgcttcgattccgacaaaaacatcagg

SEQ ID NO: 852 - DNA, 29 bp, Artificial
  primer LPXB reverse 842
ggcgcttcgattccgacaaaaacatccgg

SEQ ID NO: 853 - DNA, 29 bp, Artificial
  primer LPXB reverse 843
ggcgcttcgattccgacaaaaacatcggg

SEQ ID NO: 854 - DNA, 29 bp, Artificial
  primer LPXB reverse 844
ggcgcttcgattccgacaaaaacatctgg

SEQ ID NO: 855 - DNA, 29 bp, Artificial
  primer LPXB reverse 845
ggcgcttcgattccgacaaacacatcagg

SEQ ID NO: 856 - DNA, 29 bp, Artificial
  primer LPXB reverse 846
ggcgcttcgattccgacaaacacatccgg

SEQ ID NO: 857 - DNA, 29 bp, Artificial
  primer LPXB reverse 847
ggcgcttcgattccgacaaacacatctgg

SEQ ID NO: 858 - DNA, 29 bp, Artificial
  primer LPXB reverse 848
ggcgcttcgattccgacaaacacatcggg

SEQ ID NO: 859 - DNA, 29 bp, Artificial
  primer LPXB reverse 849
ggcgcttcgattccgacaaagacatcagg

SEQ ID NO: 860 - DNA, 29 bp, Artificial
  primer LPXB reverse 850
ggcgcttcgattccgacaaagacatccgg

SEQ ID NO: 861 - DNA, 29 bp, Artificial
  primer LPXB reverse 851
ggcgcttcgattccgacaaagacatcggg

SEQ ID NO: 862 - DNA, 29 bp, Artificial
  primer LPXB reverse 852
ggcgcttcgattccgacaaagacatctgg

SEQ ID NO: 863 - DNA, 29 bp, Artificial
  primer LPXB reverse 853
ggcgcttcgattccgacgaaaacatcagg

SEQ ID NO: 864 - DNA, 29 bp, Artificial
  primer LPXB reverse 854
ggcgcttcgattccgacgaaaacatccgg

SEQ ID NO: 865 - DNA, 29 bp, Artificial
  primer LPXB reverse 855
ggcgcttcgattccgacgaaaacatcggg

SEQ ID NO: 866 - DNA, 29 bp, Artificial
  primer LPXB reverse 856
ggcgcttcgattccgacgaaaacatctgg

SEQ ID NO: 867 - DNA, 29 bp, Artificial
  primer LPXB reverse 857
ggcgcttcgattccgacgaacacatcagg

SEQ ID NO: 868 - DNA, 29 bp, Artificial
  primer LPXB reverse 858
ggcgcttcgattccgacgaacacatccgg

SEQ ID NO: 869 - DNA, 29 bp, Artificial
  primer LPXB reverse 859
ggcgcttcgattccgacgaacacatctgg

SEQ ID NO: 870 - DNA, 29 bp, Artificial
  primer LPXB reverse 860
ggcgcttcgattccgacgaacacatcggg

SEQ ID NO: 871 - DNA, 29 bp, Artificial
  primer LPXB reverse 861
ggcgcttcgattccgacgaagacatcagg

SEQ ID NO: 872 - DNA, 29 bp, Artificial
  primer LPXB reverse 862
ggcgcttcgattccgacgaagacatccgg

SEQ ID NO: 873 - DNA, 29 bp, Artificial
  primer LPXB reverse 863
ggcgcttcgattccgacgaagacatcggg

SEQ ID NO: 874 - DNA, 29 bp, Artificial
  primer LPXB reverse 864
ggcgcttcgattccgacgaagacatctgg

SEQ ID NO: 875 - DNA, 29 bp, Artificial
  primer LPXB reverse 865
ggggcctcaataccaacaaaaacatcagg

SEQ ID NO: 876 - DNA, 29 bp, Artificial
  primer LPXB reverse 866
ggggcctcaataccaacaaaaacatccgg

SEQ ID NO: 877 - DNA, 29 bp, Artificial
  primer LPXB reverse 867
ggggcctcaataccaacaaaaacatcggg

SEQ ID NO: 878 - DNA, 29 bp, Artificial
  primer LPXB reverse 868
ggggcctcaataccaacaaaaacatctgg

SEQ ID NO: 879 - DNA, 29 bp, Artificial
  primer LPXB reverse 869
ggggcctcaataccaacaaacacatcagg

SEQ ID NO: 880 - DNA, 29 bp, Artificial
  primer LPXB reverse 870
ggggcctcaataccaacaaacacatccgg

SEQ ID NO: 881 - DNA, 29 bp, Artificial
  primer LPXB reverse 871
ggggcctcaataccaacaaacacatctgg

SEQ ID NO: 882 - DNA, 29 bp, Artificial
  primer LPXB reverse 872
ggggcctcaataccaacaaacacatcggg

SEQ ID NO: 883 - DNA, 29 bp, Artificial
  primer LPXB reverse 873
ggggcctcaataccaacaaagacatcagg

SEQ ID NO: 884 - DNA, 29 bp, Artificial
  primer LPXB reverse 874
ggggcctcaataccaacaaagacatccgg

SEQ ID NO: 885 - DNA, 29 bp, Artificial
  primer LPXB reverse 875
ggggcctcaataccaacaaagacatcggg

SEQ ID NO: 886 - DNA, 29 bp, Artificial
  primer LPXB reverse 876
ggggcctcaataccaacaaagacatctgg

SEQ ID NO: 887 - DNA, 29 bp, Artificial
  primer LPXB reverse 877
ggggcctcaataccaacgaaaacatcagg

SEQ ID NO: 888 - DNA, 29 bp, Artificial
  primer LPXB reverse 878
ggggcctcaataccaacgaaaacatccgg

SEQ ID NO: 889 - DNA, 29 bp, Artificial
  primer LPXB reverse 879
ggggcctcaataccaacgaaaacatcggg

SEQ ID NO: 890 - DNA, 29 bp, Artificial
  primer LPXB reverse 880
ggggcctcaataccaacgaaaacatctgg

SEQ ID NO: 891 - DNA, 29 bp, Artificial
  primer LPXB reverse 881
ggggcctcaataccaacgaacacatcagg

SEQ ID NO: 892 - DNA, 29 bp, Artificial
  primer LPXB reverse 882
ggggcctcaataccaacgaacacatccgg

SEQ ID NO: 893 - DNA, 29 bp, Artificial
  primer LPXB reverse 883
ggggcctcaataccaacgaacacatctgg

SEQ ID NO: 894 - DNA, 29 bp, Artificial
  primer LPXB reverse 884
ggggcctcaataccaacgaacacatcggg

SEQ ID NO: 895 - DNA, 29 bp, Artificial
  primer LPXB reverse 885
ggggcctcaataccaacgaagacatcagg

SEQ ID NO: 896 - DNA, 29 bp, Artificial
  primer LPXB reverse 886
ggggcctcaataccaacgaagacatccgg

SEQ ID NO: 897 - DNA, 29 bp, Artificial
  primer LPXB reverse 887
ggggcctcaataccaacgaagacatcggg

SEQ ID NO: 898 - DNA, 29 bp, Artificial
  primer LPXB reverse 888
ggggcctcaataccaacgaagacatctgg

SEQ ID NO: 899 - DNA, 29 bp, Artificial
  primer LPXB reverse 889
ggggcctcaatacccacaaaaacatcagg

SEQ ID NO: 900 - DNA, 29 bp, Artificial
  primer LPXB reverse 890
ggggcctcaatacccacaaaaacatccgg

SEQ ID NO: 901 - DNA, 29 bp, Artificial
  primer LPXB reverse 891
ggggcctcaatacccacaaaaacatcggg

SEQ ID NO: 902 - DNA, 29 bp, Artificial
  primer LPXB reverse 892
ggggcctcaatacccacaaaaacatctgg

SEQ ID NO: 903 - DNA, 29 bp, Artificial
  primer LPXB reverse 893
ggggcctcaatacccacaaacacatcagg

SEQ ID NO: 904 - DNA, 29 bp, Artificial
  primer LPXB reverse 894
ggggcctcaatacccacaaacacatccgg

SEQ ID NO: 905 - DNA, 29 bp, Artificial
  primer LPXB reverse 895
ggggcctcaatacccacaaacacatctgg

SEQ ID NO: 906 - DNA, 29 bp, Artificial
  primer LPXB reverse 896
ggggcctcaatacccacaaacacatcggg

SEQ ID NO: 907 - DNA, 29 bp, Artificial
  primer LPXB reverse 897
ggggcctcaatacccacaaagacatcagg

SEQ ID NO: 908 - DNA, 29 bp, Artificial
  primer LPXB reverse 898
ggggcctcaatacccacaaagacatccgg

SEQ ID NO: 909 - DNA, 29 bp, Artificial
  primer LPXB reverse 899
ggggcctcaatacccacaaagacatcggg

SEQ ID NO: 910 - DNA, 29 bp, Artificial
  primer LPXB reverse 900
ggggcctcaatacccacaaagacatctgg

SEQ ID NO: 911 - DNA, 29 bp, Artificial
  primer LPXB reverse 901
ggggcctcaatacccacgaaaacatcagg

SEQ ID NO: 912 - DNA, 29 bp, Artificial
  primer LPXB reverse 902
ggggcctcaatacccacgaaaacatccgg

SEQ ID NO: 913 - DNA, 29 bp, Artificial
  primer LPXB reverse 903
ggggcctcaatacccacgaaaacatcggg

SEQ ID NO: 914 - DNA, 29 bp, Artificial
  primer LPXB reverse 904
ggggcctcaatacccacgaaaacatctgg

SEQ ID NO: 915 - DNA, 29 bp, Artificial
  primer LPXB reverse 905
ggggcctcaatacccacgaacacatcagg

SEQ ID NO: 916 - DNA, 29 bp, Artificial
  primer LPXB reverse 906
ggggcctcaatacccacgaacacatccgg

SEQ ID NO: 917 - DNA, 29 bp, Artificial
  primer LPXB reverse 907
ggggcctcaatacccacgaacacatctgg

SEQ ID NO: 918 - DNA, 29 bp, Artificial
  primer LPXB reverse 908
ggggcctcaatacccacgaacacatcggg

SEQ ID NO: 919 - DNA, 29 bp, Artificial
  primer LPXB reverse 909
ggggcctcaatacccacgaagacatcagg

SEQ ID NO: 920 - DNA, 29 bp, Artificial
  primer LPXB reverse 910
ggggcctcaatacccacgaagacatccgg

SEQ ID NO: 921 - DNA, 29 bp, Artificial
  primer LPXB reverse 911
ggggcctcaatacccacgaagacatcggg

SEQ ID NO: 922 - DNA, 29 bp, Artificial
  primer LPXB reverse 912
ggggcctcaatacccacgaagacatctgg

SEQ ID NO: 923 - DNA, 29 bp, Artificial
  primer LPXB reverse 913
ggggcctcaataccgacaaaaacatcagg

SEQ ID NO: 924 - DNA, 29 bp, Artificial
  primer LPXB reverse 914
ggggcctcaataccgacaaaaacatccgg

SEQ ID NO: 925 - DNA, 29 bp, Artificial
  primer LPXB reverse 915
ggggcctcaataccgacaaaaacatcggg

SEQ ID NO: 926 - DNA, 29 bp, Artificial
  primer LPXB reverse 916
ggggcctcaataccgacaaaaacatctgg

SEQ ID NO: 927 - DNA, 29 bp, Artificial
  primer LPXB reverse 917
ggggcctcaataccgacaaacacatcagg

SEQ ID NO: 928 - DNA, 29 bp, Artificial
  primer LPXB reverse 918
ggggcctcaataccgacaaacacatccgg

SEQ ID NO: 929 - DNA, 29 bp, Artificial
  primer LPXB reverse 919
ggggcctcaataccgacaaacacatctgg

SEQ ID NO: 930 - DNA, 29 bp, Artificial
  primer LPXB reverse 920
ggggcctcaataccgacaaacacatcggg

SEQ ID NO: 931 - DNA, 29 bp, Artificial
  primer LPXB reverse 921
ggggcctcaataccgacaaagacatcagg

SEQ ID NO: 932 - DNA, 29 bp, Artificial
  primer LPXB reverse 922
ggggcctcaataccgacaaagacatccgg

SEQ ID NO: 933 - DNA, 29 bp, Artificial
  primer LPXB reverse 923
ggggcctcaataccgacaaagacatcggg

SEQ ID NO: 934 - DNA, 29 bp, Artificial
  primer LPXB reverse 924
ggggcctcaataccgacaaagacatctgg

SEQ ID NO: 935 - DNA, 29 bp, Artificial
  primer LPXB reverse 925
ggggcctcaataccgacgaaaacatcagg

SEQ ID NO: 936 - DNA, 29 bp, Artificial
  primer LPXB reverse 926
ggggcctcaataccgacgaaaacatccgg

SEQ ID NO: 937 - DNA, 29 bp, Artificial
  primer LPXB reverse 927
ggggcctcaataccgacgaaaacatcggg

SEQ ID NO: 938 - DNA, 29 bp, Artificial
  primer LPXB reverse 928
ggggcctcaataccgacgaaaacatctgg

SEQ ID NO: 939 - DNA, 29 bp, Artificial
  primer LPXB reverse 929
ggggcctcaataccgacgaacacatcagg

SEQ ID NO: 940 - DNA, 29 bp, Artificial
  primer LPXB reverse 930
ggggcctcaataccgacgaacacatccgg

SEQ ID NO: 941 - DNA, 29 bp, Artificial
  primer LPXB reverse 931
ggggcctcaataccgacgaacacatctgg

SEQ ID NO: 942 - DNA, 29 bp, Artificial
  primer LPXB reverse 932
ggggcctcaataccgacgaacacatcggg

SEQ ID NO: 943 - DNA, 29 bp, Artificial
  primer LPXB reverse 933
ggggcctcaataccgacgaagacatcagg

SEQ ID NO: 944 - DNA, 29 bp, Artificial
  primer LPXB reverse 934
ggggcctcaataccgacgaagacatccgg

SEQ ID NO: 945 - DNA, 29 bp, Artificial
  primer LPXB reverse 935
ggggcctcaataccgacgaagacatcggg

SEQ ID NO: 946 - DNA, 29 bp, Artificial
  primer LPXB reverse 936
ggggcctcaataccgacgaagacatctgg

SEQ ID NO: 947 - DNA, 29 bp, Artificial
  primer LPXB reverse 937
ggggcctcaatcccaacaaaaacatcagg

SEQ ID NO: 948 - DNA, 29 bp, Artificial
  primer LPXB reverse 938
ggggcctcaatcccaacaaaaacatccgg

SEQ ID NO: 949 - DNA, 29 bp, Artificial
  primer LPXB reverse 939
ggggcctcaatcccaacaaaaacatcggg

SEQ ID NO: 950 - DNA, 29 bp, Artificial
  primer LPXB reverse 940
ggggcctcaatcccaacaaaaacatctgg

SEQ ID NO: 951 - DNA, 29 bp, Artificial
  primer LPXB reverse 941
ggggcctcaatcccaacaaacacatcagg

SEQ ID NO: 952 - DNA, 29 bp, Artificial
  primer LPXB reverse 942
ggggcctcaatcccaacaaacacatccgg

SEQ ID NO: 953 - DNA, 29 bp, Artificial
  primer LPXB reverse 943
ggggcctcaatcccaacaaacacatctgg

SEQ ID NO: 954 - DNA, 29 bp, Artificial
  primer LPXB reverse 944
ggggcctcaatcccaacaaacacatcggg

SEQ ID NO: 955 - DNA, 29 bp, Artificial
  primer LPXB reverse 945
ggggcctcaatcccaacaaagacatcagg

SEQ ID NO: 956 - DNA, 29 bp, Artificial
  primer LPXB reverse 946
ggggcctcaatcccaacaaagacatccgg

SEQ ID NO: 957 - DNA, 29 bp, Artificial
  primer LPXB reverse 947
ggggcctcaatcccaacaaagacatcggg

SEQ ID NO: 958 - DNA, 29 bp, Artificial
  primer LPXB reverse 948
ggggcctcaatcccaacaaagacatctgg

SEQ ID NO: 959 - DNA, 29 bp, Artificial
  primer LPXB reverse 949
ggggcctcaatcccaacgaaaacatcagg

SEQ ID NO: 960 - DNA, 29 bp, Artificial
  primer LPXB reverse 950
ggggcctcaatcccaacgaaaacatccgg

SEQ ID NO: 961 - DNA, 29 bp, Artificial
  primer LPXB reverse 951
ggggcctcaatcccaacgaaaacatcggg

SEQ ID NO: 962 - DNA, 29 bp, Artificial
  primer LPXB reverse 952
ggggcctcaatcccaacgaaaacatctgg

SEQ ID NO: 963 - DNA, 29 bp, Artificial
  primer LPXB reverse 953
ggggcctcaatcccaacgaacacatcagg

SEQ ID NO: 964 - DNA, 29 bp, Artificial
  primer LPXB reverse 954
ggggcctcaatcccaacgaacacatccgg

SEQ ID NO: 965 - DNA, 29 bp, Artificial
  primer LPXB reverse 955
ggggcctcaatcccaacgaacacatctgg

SEQ ID NO: 966 - DNA, 29 bp, Artificial
  primer LPXB reverse 956
ggggcctcaatcccaacgaacacatcggg

SEQ ID NO: 967 - DNA, 29 bp, Artificial
  primer LPXB reverse 957
ggggcctcaatcccaacgaagacatcagg

SEQ ID NO: 968 - DNA, 29 bp, Artificial
  primer LPXB reverse 958
ggggcctcaatcccaacgaagacatccgg

SEQ ID NO: 969 - DNA, 29 bp, Artificial
  primer LPXB reverse 959
ggggcctcaatcccaacgaagacatcggg

SEQ ID NO: 970 - DNA, 29 bp, Artificial
  primer LPXB reverse 960
ggggcctcaatcccaacgaagacatctgg

SEQ ID NO: 971 - DNA, 29 bp, Artificial
  primer LPXB reverse 961
ggggcctcaatccccacaaaaacatcagg

SEQ ID NO: 972 - DNA, 29 bp, Artificial
  primer LPXB reverse 962
ggggcctcaatccccacaaaaacatccgg

SEQ ID NO: 973 - DNA, 29 bp, Artificial
  primer LPXB reverse 963
ggggcctcaatccccacaaaaacatcggg

SEQ ID NO: 974 - DNA, 29 bp, Artificial
  primer LPXB reverse 964
ggggcctcaatccccacaaaaacatctgg

SEQ ID NO: 975 - DNA, 29 bp, Artificial
  primer LPXB reverse 965
ggggcctcaatccccacaaacacatcagg

SEQ ID NO: 976 - DNA, 29 bp, Artificial
  primer LPXB reverse 966
ggggcctcaatccccacaaacacatccgg

SEQ ID NO: 977 - DNA, 29 bp, Artificial
  primer LPXB reverse 967
ggggcctcaatccccacaaacacatctgg

SEQ ID NO: 978 - DNA, 29 bp, Artificial
  primer LPXB reverse 968
ggggcctcaatccccacaaacacatcggg

SEQ ID NO: 979 - DNA, 29 bp, Artificial
  primer LPXB reverse 969
ggggcctcaatccccacaaagacatcagg

SEQ ID NO: 980 - DNA, 29 bp, Artificial
  primer LPXB reverse 970
ggggcctcaatccccacaaagacatccgg

SEQ ID NO: 981 - DNA, 29 bp, Artificial
  primer LPXB reverse 971
ggggcctcaatccccacaaagacatcggg

SEQ ID NO: 982 - DNA, 29 bp, Artificial
  primer LPXB reverse 972
ggggcctcaatccccacaaagacatctgg

SEQ ID NO: 983 - DNA, 29 bp, Artificial
  primer LPXB reverse 973
ggggcctcaatccccacgaaaacatcagg

SEQ ID NO: 984 - DNA, 29 bp, Artificial
  primer LPXB reverse 974
ggggcctcaatccccacgaaaacatccgg

SEQ ID NO: 985 - DNA, 29 bp, Artificial
  primer LPXB reverse 975
ggggcctcaatccccacgaaaacatcggg

SEQ ID NO: 986 - DNA, 29 bp, Artificial
  primer LPXB reverse 976
ggggcctcaatccccacgaaaacatctgg

SEQ ID NO: 987 - DNA, 29 bp, Artificial
  primer LPXB reverse 977
ggggcctcaatccccacgaacacatcagg

SEQ ID NO: 988 - DNA, 29 bp, Artificial
  primer LPXB reverse 978
ggggcctcaatccccacgaacacatccgg

SEQ ID NO: 989 - DNA, 29 bp, Artificial
  primer LPXB reverse 979
ggggcctcaatccccacgaacacatctgg

SEQ ID NO: 990 - DNA, 29 bp, Artificial
  primer LPXB reverse 980
ggggcctcaatccccacgaacacatcggg

SEQ ID NO: 991 - DNA, 29 bp, Artificial
  primer LPXB reverse 981
ggggcctcaatccccacgaagacatcagg

SEQ ID NO: 992 - DNA, 29 bp, Artificial
  primer LPXB reverse 982
ggggcctcaatccccacgaagacatccgg

SEQ ID NO: 993 - DNA, 29 bp, Artificial
  primer LPXB reverse 983
ggggcctcaatccccacgaagacatcggg

SEQ ID NO: 994 - DNA, 29 bp, Artificial
  primer LPXB reverse 984
ggggcctcaatccccacgaagacatctgg

SEQ ID NO: 995 - DNA, 29 bp, Artificial
  primer LPXB reverse 985
ggggcctcaatcccgacaaaaacatcagg

SEQ ID NO: 996 - DNA, 29 bp, Artificial
  primer LPXB reverse 986
ggggcctcaatcccgacaaaaacatccgg

SEQ ID NO: 997 - DNA, 29 bp, Artificial
  primer LPXB reverse 987
ggggcctcaatcccgacaaaaacatcggg

SEQ ID NO: 998 - DNA, 29 bp, Artificial
  primer LPXB reverse 988
ggggcctcaatcccgacaaaaacatctgg

SEQ ID NO: 999 - DNA, 29 bp, Artificial
  primer LPXB reverse 989
ggggcctcaatcccgacaaacacatcagg

SEQ ID NO: 1000 - DNA, 29 bp, Artificial
  primer LPXB reverse 990
ggggcctcaatcccgacaaacacatccgg

SEQ ID NO: 1001 - DNA, 29 bp, Artificial
  primer LPXB reverse 991
ggggcctcaatcccgacaaacacatctgg

SEQ ID NO: 1002 - DNA, 29 bp, Artificial
  primer LPXB reverse 992
ggggcctcaatcccgacaaacacatcggg

SEQ ID NO: 1003 - DNA, 29 bp, Artificial
  primer LPXB reverse 993
ggggcctcaatcccgacaaagacatcagg

SEQ ID NO: 1004 - DNA, 29 bp, Artificial
  primer LPXB reverse 994
ggggcctcaatcccgacaaagacatccgg

SEQ ID NO: 1005 - DNA, 29 bp, Artificial
  primer LPXB reverse 995
ggggcctcaatcccgacaaagacatcggg

SEQ ID NO: 1006 - DNA, 29 bp, Artificial
  primer LPXB reverse 996
ggggcctcaatcccgacaaagacatctgg

SEQ ID NO: 1007 - DNA, 29 bp, Artificial
  primer LPXB reverse 997
ggggcctcaatcccgacgaaaacatcagg

SEQ ID NO: 1008 - DNA, 29 bp, Artificial
  primer LPXB reverse 998
ggggcctcaatcccgacgaaaacatccgg

SEQ ID NO: 1009 - DNA, 29 bp, Artificial
  primer LPXB reverse 999
ggggcctcaatcccgacgaaaacatcggg

SEQ ID NO: 1010 - DNA, 29 bp, Artificial
  primer LPXB reverse 1000
ggggcctcaatcccgacgaaaacatctgg

SEQ ID NO: 1011 - DNA, 29 bp, Artificial
  primer LPXB reverse 1001
ggggcctcaatcccgacgaacacatcagg

SEQ ID NO: 1012 - DNA, 29 bp, Artificial
  primer LPXB reverse 1002
ggggcctcaatcccgacgaacacatccgg

SEQ ID NO: 1013 - DNA, 29 bp, Artificial
  primer LPXB reverse 1003
ggggcctcaatcccgacgaacacatctgg

SEQ ID NO: 1014 - DNA, 29 bp, Artificial
  primer LPXB reverse 1004
ggggcctcaatcccgacgaacacatcggg

SEQ ID NO: 1015 - DNA, 29 bp, Artificial
  primer LPXB reverse 1005
ggggcctcaatcccgacgaagacatcagg

SEQ ID NO: 1016 - DNA, 29 bp, Artificial
  primer LPXB reverse 1006
ggggcctcaatcccgacgaagacatccgg

SEQ ID NO: 1017 - DNA, 29 bp, Artificial
  primer LPXB reverse 1007
ggggcctcaatcccgacgaagacatcggg

SEQ ID NO: 1018 - DNA, 29 bp, Artificial
  primer LPXB reverse 1008
ggggcctcaatcccgacgaagacatctgg

SEQ ID NO: 1019 - DNA, 29 bp, Artificial
  primer LPXB reverse 1009
ggggcctcaattccaacaaaaacatcagg

SEQ ID NO: 1020 - DNA, 29 bp, Artificial
  primer LPXB reverse 1010
ggggcctcaattccaacaaaaacatccgg

SEQ ID NO: 1021 - DNA, 29 bp, Artificial
  primer LPXB reverse 1011
ggggcctcaattccaacaaaaacatcggg

SEQ ID NO: 1022 - DNA, 29 bp, Artificial
  primer LPXB reverse 1012
ggggcctcaattccaacaaaaacatctgg

SEQ ID NO: 1023 - DNA, 29 bp, Artificial
  primer LPXB reverse 1013
ggggcctcaattccaacaaacacatcagg

SEQ ID NO: 1024 - DNA, 29 bp, Artificial
  primer LPXB reverse 1014
ggggcctcaattccaacaaacacatccgg

SEQ ID NO: 1025 - DNA, 29 bp, Artificial
  primer LPXB reverse 1015
ggggcctcaattccaacaaacacatctgg

SEQ ID NO: 1026 - DNA, 29 bp, Artificial
  primer LPXB reverse 1016
ggggcctcaattccaacaaacacatcggg

SEQ ID NO: 1027 - DNA, 29 bp, Artificial
  primer LPXB reverse 1017
ggggcctcaattccaacaaagacatcagg

SEQ ID NO: 1028 - DNA, 29 bp, Artificial
  primer LPXB reverse 1018
ggggcctcaattccaacaaagacatccgg

SEQ ID NO: 1029 - DNA, 29 bp, Artificial
  primer LPXB reverse 1019
ggggcctcaattccaacaaagacatcggg

SEQ ID NO: 1030 - DNA, 29 bp, Artificial
  primer LPXB reverse 1020
ggggcctcaattccaacaaagacatctgg

SEQ ID NO: 1031 - DNA, 29 bp, Artificial
  primer LPXB reverse 1021
ggggcctcaattccaacgaaaacatcagg

SEQ ID NO: 1032 - DNA, 29 bp, Artificial
  primer LPXB reverse 1022
ggggcctcaattccaacgaaaacatccgg

SEQ ID NO: 1033 - DNA, 29 bp, Artificial
  primer LPXB reverse 1023
ggggcctcaattccaacgaaaacatcggg

SEQ ID NO: 1034 - DNA, 29 bp, Artificial
  primer LPXB reverse 1024
ggggcctcaattccaacgaaaacatctgg

SEQ ID NO: 1035 - DNA, 29 bp, Artificial
  primer LPXB reverse 1025
ggggcctcaattccaacgaacacatcagg

SEQ ID NO: 1036 - DNA, 29 bp, Artificial
  primer LPXB reverse 1026
ggggcctcaattccaacgaacacatccgg

SEQ ID NO: 1037 - DNA, 29 bp, Artificial
  primer LPXB reverse 1027
ggggcctcaattccaacgaacacatctgg

SEQ ID NO: 1038 - DNA, 29 bp, Artificial
  primer LPXB reverse 1028
ggggcctcaattccaacgaacacatcggg

SEQ ID NO: 1039 - DNA, 29 bp, Artificial
  primer LPXB reverse 1029
ggggcctcaattccaacgaagacatcagg

SEQ ID NO: 1040 - DNA, 29 bp, Artificial
  primer LPXB reverse 1030
ggggcctcaattccaacgaagacatccgg

SEQ ID NO: 1041 - DNA, 29 bp, Artificial
  primer LPXB reverse 1031
ggggcctcaattccaacgaagacatcggg

SEQ ID NO: 1042 - DNA, 29 bp, Artificial
  primer LPXB reverse 1032
ggggcctcaattccaacgaagacatctgg

SEQ ID NO: 1043 - DNA, 29 bp, Artificial
  primer LPXB reverse 1033
ggggcctcaattcccacaaaaacatcagg

SEQ ID NO: 1044 - DNA, 29 bp, Artificial
  primer LPXB reverse 1034
ggggcctcaattcccacaaaaacatccgg

SEQ ID NO: 1045 - DNA, 29 bp, Artificial
  primer LPXB reverse 1035
ggggcctcaattcccacaaaaacatcggg

SEQ ID NO: 1046 - DNA, 29 bp, Artificial
  primer LPXB reverse 1036
ggggcctcaattcccacaaaaacatctgg

SEQ ID NO: 1047 - DNA, 29 bp, Artificial
  primer LPXB reverse 1037
ggggcctcaattcccacaaacacatcagg

SEQ ID NO: 1048 - DNA, 29 bp, Artificial
  primer LPXB reverse 1038
ggggcctcaattcccacaaacacatccgg

SEQ ID NO: 1049 - DNA, 29 bp, Artificial
  primer LPXB reverse 1039
ggggcctcaattcccacaaacacatctgg

SEQ ID NO: 1050 - DNA, 29 bp, Artificial
  primer LPXB reverse 1040
ggggcctcaattcccacaaacacatcggg

SEQ ID NO: 1051 - DNA, 29 bp, Artificial
  primer LPXB reverse 1041
ggggcctcaattcccacaaagacatcagg

SEQ ID NO: 1052 - DNA, 29 bp, Artificial
  primer LPXB reverse 1042
ggggcctcaattcccacaaagacatccgg

SEQ ID NO: 1053 - DNA, 29 bp, Artificial
  primer LPXB reverse 1043
ggggcctcaattcccacaaagacatcggg

SEQ ID NO: 1054 - DNA, 29 bp, Artificial
  primer LPXB reverse 1044
ggggcctcaattcccacaaagacatctgg

SEQ ID NO: 1055 - DNA, 29 bp, Artificial
  primer LPXB reverse 1045
ggggcctcaattcccacgaaaacatcagg

SEQ ID NO: 1056 - DNA, 29 bp, Artificial
  primer LPXB reverse 1046
ggggcctcaattcccacgaaaacatccgg

SEQ ID NO: 1057 - DNA, 29 bp, Artificial
  primer LPXB reverse 1047
ggggcctcaattcccacgaaaacatcggg

SEQ ID NO: 1058 - DNA, 29 bp, Artificial
  primer LPXB reverse 1048
ggggcctcaattcccacgaaaacatctgg

SEQ ID NO: 1059 - DNA, 29 bp, Artificial
  primer LPXB reverse 1049
ggggcctcaattcccacgaacacatcagg

SEQ ID NO: 1060 - DNA, 29 bp, Artificial
  primer LPXB reverse 1050
ggggcctcaattcccacgaacacatccgg

SEQ ID NO: 1061 - DNA, 29 bp, Artificial
  primer LPXB reverse 1051
ggggcctcaattcccacgaacacatctgg

SEQ ID NO: 1062 - DNA, 29 bp, Artificial
  primer LPXB reverse 1052
ggggcctcaattcccacgaacacatcggg

SEQ ID NO: 1063 - DNA, 29 bp, Artificial
  primer LPXB reverse 1053
ggggcctcaattcccacgaagacatcagg

SEQ ID NO: 1064 - DNA, 29 bp, Artificial
  primer LPXB reverse 1054
ggggcctcaattcccacgaagacatccgg

SEQ ID NO: 1065 - DNA, 29 bp, Artificial
  primer LPXB reverse 1055
ggggcctcaattcccacgaagacatcggg

SEQ ID NO: 1066 - DNA, 29 bp, Artificial
  primer LPXB reverse 1056
ggggcctcaattcccacgaagacatctgg

SEQ ID NO: 1067 - DNA, 29 bp, Artificial
  primer LPXB reverse 1057
ggggcctcaattccgacaaaaacatcagg

SEQ ID NO: 1068 - DNA, 29 bp, Artificial
  primer LPXB reverse 1058
ggggcctcaattccgacaaaaacatccgg

SEQ ID NO: 1069 - DNA, 29 bp, Artificial
  primer LPXB reverse 1059
ggggcctcaattccgacaaaaacatcggg

SEQ ID NO: 1070 - DNA, 29 bp, Artificial
  primer LPXB reverse 1060
ggggcctcaattccgacaaaaacatctgg

SEQ ID NO: 1071 - DNA, 29 bp, Artificial
  primer LPXB reverse 1061
ggggcctcaattccgacaaacacatcagg

SEQ ID NO: 1072 - DNA, 29 bp, Artificial
  primer LPXB reverse 1062
ggggcctcaattccgacaaacacatccgg

SEQ ID NO: 1073 - DNA, 29 bp, Artificial
  primer LPXB reverse 1063
ggggcctcaattccgacaaacacatctgg

SEQ ID NO: 1074 - DNA, 29 bp, Artificial
  primer LPXB reverse 1064
ggggcctcaattccgacaaacacatcggg

SEQ ID NO: 1075 - DNA, 29 bp, Artificial
  primer LPXB reverse 1065
ggggcctcaattccgacaaagacatcagg

SEQ ID NO: 1076 - DNA, 29 bp, Artificial
  primer LPXB reverse 1066
ggggcctcaattccgacaaagacatccgg

SEQ ID NO: 1077 - DNA, 29 bp, Artificial
  primer LPXB reverse 1067
ggggcctcaattccgacaaagacatcggg

SEQ ID NO: 1078 - DNA, 29 bp, Artificial
  primer LPXB reverse 1068
ggggcctcaattccgacaaagacatctgg

SEQ ID NO: 1079 - DNA, 29 bp, Artificial
  primer LPXB reverse 1069
ggggcctcaattccgacgaaaacatcagg

SEQ ID NO: 1080 - DNA, 29 bp, Artificial
  primer LPXB reverse 1070
ggggcctcaattccgacgaaaacatccgg

SEQ ID NO: 1081 - DNA, 29 bp, Artificial
  primer LPXB reverse 1071
ggggcctcaattccgacgaaaacatcggg

SEQ ID NO: 1082 - DNA, 29 bp, Artificial
  primer LPXB reverse 1072
ggggcctcaattccgacgaaaacatctgg

SEQ ID NO: 1083 - DNA, 29 bp, Artificial
  primer LPXB reverse 1073
ggggcctcaattccgacgaacacatcagg

SEQ ID NO: 1084 - DNA, 29 bp, Artificial
  primer LPXB reverse 1074
ggggcctcaattccgacgaacacatccgg

SEQ ID NO: 1085 - DNA, 29 bp, Artificial
  primer LPXB reverse 1075
ggggcctcaattccgacgaacacatctgg

SEQ ID NO: 1086 - DNA, 29 bp, Artificial
  primer LPXB reverse 1076
ggggcctcaattccgacgaacacatcggg

SEQ ID NO: 1087 - DNA, 29 bp, Artificial
  primer LPXB reverse 1077
ggggcctcaattccgacgaagacatcagg

SEQ ID NO: 1088 - DNA, 29 bp, Artificial
  primer LPXB reverse 1078
ggggcctcaattccgacgaagacatccgg

SEQ ID NO: 1089 - DNA, 29 bp, Artificial
  primer LPXB reverse 1079
ggggcctcaattccgacgaagacatcggg

SEQ ID NO: 1090 - DNA, 29 bp, Artificial
  primer LPXB reverse 1080
ggggcctcaattccgacgaagacatctgg

SEQ ID NO: 1091 - DNA, 29 bp, Artificial
  primer LPXB reverse 1081
ggggcctcgataccaacaaaaacatcagg

SEQ ID NO: 1092 - DNA, 29 bp, Artificial
  primer LPXB reverse 1082
ggggcctcgataccaacaaaaacatccgg

SEQ ID NO: 1093 - DNA, 29 bp, Artificial
  primer LPXB reverse 1083
ggggcctcgataccaacaaaaacatcggg

SEQ ID NO: 1094 - DNA, 29 bp, Artificial
  primer LPXB reverse 1084
ggggcctcgataccaacaaaaacatctgg

SEQ ID NO: 1095 - DNA, 29 bp, Artificial
  primer LPXB reverse 1085
ggggcctcgataccaacaaacacatcagg

SEQ ID NO: 1096 - DNA, 29 bp, Artificial
  primer LPXB reverse 1086
ggggcctcgataccaacaaacacatccgg

SEQ ID NO: 1097 - DNA, 29 bp, Artificial
  primer LPXB reverse 1087
ggggcctcgataccaacaaacacatctgg

SEQ ID NO: 1098 - DNA, 29 bp, Artificial
  primer LPXB reverse 1088
ggggcctcgataccaacaaacacatcggg

SEQ ID NO: 1099 - DNA, 29 bp, Artificial
  primer LPXB reverse 1089
ggggcctcgataccaacaaagacatcagg

SEQ ID NO: 1100 - DNA, 29 bp, Artificial
  primer LPXB reverse 1090
ggggcctcgataccaacaaagacatccgg

SEQ ID NO: 1101 - DNA, 29 bp, Artificial
  primer LPXB reverse 1091
ggggcctcgataccaacaaagacatcggg

SEQ ID NO: 1102 - DNA, 29 bp, Artificial
  primer LPXB reverse 1092
ggggcctcgataccaacaaagacatctgg

SEQ ID NO: 1103 - DNA, 29 bp, Artificial
  primer LPXB reverse 1093
ggggcctcgataccaacgaaaacatcagg

SEQ ID NO: 1104 - DNA, 29 bp, Artificial
  primer LPXB reverse 1094
ggggcctcgataccaacgaaaacatccgg

SEQ ID NO: 1105 - DNA, 29 bp, Artificial
  primer LPXB reverse 1095
ggggcctcgataccaacgaaaacatcggg

SEQ ID NO: 1106 - DNA, 29 bp, Artificial
  primer LPXB reverse 1096
ggggcctcgataccaacgaaaacatctgg

SEQ ID NO: 1107 - DNA, 29 bp, Artificial
  primer LPXB reverse 1097
ggggcctcgataccaacgaacacatcagg

SEQ ID NO: 1108 - DNA, 29 bp, Artificial
  primer LPXB reverse 1098
ggggcctcgataccaacgaacacatccgg

SEQ ID NO: 1109 - DNA, 29 bp, Artificial
  primer LPXB reverse 1099
ggggcctcgataccaacgaacacatctgg

SEQ ID NO: 1110 - DNA, 29 bp, Artificial
  primer LPXB reverse 1100
ggggcctcgataccaacgaacacatcggg

SEQ ID NO: 1111 - DNA, 29 bp, Artificial
  primer LPXB reverse 1101
ggggcctcgataccaacgaagacatcagg

SEQ ID NO: 1112 - DNA, 29 bp, Artificial
  primer LPXB reverse 1102
ggggcctcgataccaacgaagacatccgg

SEQ ID NO: 1113 - DNA, 29 bp, Artificial
  primer LPXB reverse 1103
ggggcctcgataccaacgaagacatcggg

SEQ ID NO: 1114 - DNA, 29 bp, Artificial
  primer LPXB reverse 1104
ggggcctcgataccaacgaagacatctgg

SEQ ID NO: 1115 - DNA, 29 bp, Artificial
  primer LPXB reverse 1105
ggggcctcgatacccacaaaaacatcagg

SEQ ID NO: 1116 - DNA, 29 bp, Artificial
  primer LPXB reverse 1106
ggggcctcgatacccacaaaaacatccgg

SEQ ID NO: 1117 - DNA, 29 bp, Artificial
  primer LPXB reverse 1107
ggggcctcgatacccacaaaaacatcggg

SEQ ID NO: 1118 - DNA, 29 bp, Artificial
  primer LPXB reverse 1108
ggggcctcgatacccacaaaaacatctgg

SEQ ID NO: 1119 - DNA, 29 bp, Artificial
  primer LPXB reverse 1109
ggggcctcgatacccacaaacacatcagg

SEQ ID NO: 1120 - DNA, 29 bp, Artificial
  primer LPXB reverse 1110
ggggcctcgatacccacaaacacatccgg

SEQ ID NO: 1121 - DNA, 29 bp, Artificial
  primer LPXB reverse 1111
ggggcctcgatacccacaaacacatctgg

SEQ ID NO: 1122 - DNA, 29 bp, Artificial
  primer LPXB reverse 1112
ggggcctcgatacccacaaacacatcggg

SEQ ID NO: 1123 - DNA, 29 bp, Artificial
  primer LPXB reverse 1113
ggggcctcgatacccacaaagacatcagg

SEQ ID NO: 1124 - DNA, 29 bp, Artificial
  primer LPXB reverse 1114
ggggcctcgatacccacaaagacatccgg

SEQ ID NO: 1125 - DNA, 29 bp, Artificial
  primer LPXB reverse 1115
ggggcctcgatacccacaaagacatcggg

SEQ ID NO: 1126 - DNA, 29 bp, Artificial
  primer LPXB reverse 1116
ggggcctcgatacccacaaagacatctgg

SEQ ID NO: 1127 - DNA, 29 bp, Artificial
  primer LPXB reverse 1117
ggggcctcgatacccacgaaaacatcagg

SEQ ID NO: 1128 - DNA, 29 bp, Artificial
  primer LPXB reverse 1118
ggggcctcgatacccacgaaaacatccgg

SEQ ID NO: 1129 - DNA, 29 bp, Artificial
  primer LPXB reverse 1119
ggggcctcgatacccacgaaaacatcggg

SEQ ID NO: 1130 - DNA, 29 bp, Artificial
  primer LPXB reverse 1120
ggggcctcgatacccacgaaaacatctgg

SEQ ID NO: 1131 - DNA, 29 bp, Artificial
  primer LPXB reverse 1121
ggggcctcgatacccacgaacacatcagg

SEQ ID NO: 1132 - DNA, 29 bp, Artificial
  primer LPXB reverse 1122
ggggcctcgatacccacgaacacatccgg

SEQ ID NO: 1133 - DNA, 29 bp, Artificial
  primer LPXB reverse 1123
ggggcctcgatacccacgaacacatctgg

SEQ ID NO: 1134 - DNA, 29 bp, Artificial
  primer LPXB reverse 1124
ggggcctcgatacccacgaacacatcggg

SEQ ID NO: 1135 - DNA, 29 bp, Artificial
  primer LPXB reverse 1125
ggggcctcgatacccacgaagacatcagg

SEQ ID NO: 1136 - DNA, 29 bp, Artificial
  primer LPXB reverse 1126
ggggcctcgatacccacgaagacatccgg

SEQ ID NO: 1137 - DNA, 29 bp, Artificial
  primer LPXB reverse 1127
ggggcctcgatacccacgaagacatcggg

SEQ ID NO: 1138 - DNA, 29 bp, Artificial
  primer LPXB reverse 1128
ggggcctcgatacccacgaagacatctgg

SEQ ID NO: 1139 - DNA, 29 bp, Artificial
  primer LPXB reverse 1129
ggggcctcgataccgacaaaaacatcagg

SEQ ID NO: 1140 - DNA, 29 bp, Artificial
  primer LPXB reverse 1130
ggggcctcgataccgacaaaaacatccgg

SEQ ID NO: 1141 - DNA, 29 bp, Artificial
  primer LPXB reverse 1131
ggggcctcgataccgacaaaaacatcggg

SEQ ID NO: 1142 - DNA, 29 bp, Artificial
  primer LPXB reverse 1132
ggggcctcgataccgacaaaaacatctgg

SEQ ID NO: 1143 - DNA, 29 bp, Artificial
  primer LPXB reverse 1133
ggggcctcgataccgacaaacacatcagg

SEQ ID NO: 1144 - DNA, 29 bp, Artificial
  primer LPXB reverse 1134
ggggcctcgataccgacaaacacatccgg

SEQ ID NO: 1145 - DNA, 29 bp, Artificial
  primer LPXB reverse 1135
ggggcctcgataccgacaaacacatctgg

SEQ ID NO: 1146 - DNA, 29 bp, Artificial
  primer LPXB reverse 1136
ggggcctcgataccgacaaacacatcggg

SEQ ID NO: 1147 - DNA, 29 bp, Artificial
  primer LPXB reverse 1137
ggggcctcgataccgacaaagacatcagg

SEQ ID NO: 1148 - DNA, 29 bp, Artificial
  primer LPXB reverse 1138
ggggcctcgataccgacaaagacatccgg

SEQ ID NO: 1149 - DNA, 29 bp, Artificial
  primer LPXB reverse 1139
ggggcctcgataccgacaaagacatcggg

SEQ ID NO: 1150 - DNA, 29 bp, Artificial
  primer LPXB reverse 1140
ggggcctcgataccgacaaagacatctgg

SEQ ID NO: 1151 - DNA, 29 bp, Artificial
  primer LPXB reverse 1141
ggggcctcgataccgacgaaaacatcagg

SEQ ID NO: 1152 - DNA, 29 bp, Artificial
  primer LPXB reverse 1142
ggggcctcgataccgacgaaaacatccgg

SEQ ID NO: 1153 - DNA, 29 bp, Artificial
  primer LPXB reverse 1143
ggggcctcgataccgacgaaaacatcggg

SEQ ID NO: 1154 - DNA, 29 bp, Artificial
  primer LPXB reverse 1144
ggggcctcgataccgacgaaaacatctgg

SEQ ID NO: 1155 - DNA, 29 bp, Artificial
  primer LPXB reverse 1145
ggggcctcgataccgacgaacacatcagg

SEQ ID NO: 1156 - DNA, 29 bp, Artificial
  primer LPXB reverse 1146
ggggcctcgataccgacgaacacatccgg

SEQ ID NO: 1157 - DNA, 29 bp, Artificial
  primer LPXB reverse 1147
ggggcctcgataccgacgaacacatctgg

SEQ ID NO: 1158 - DNA, 29 bp, Artificial
  primer LPXB reverse 1148
ggggcctcgataccgacgaacacatcggg

SEQ ID NO: 1159 - DNA, 29 bp, Artificial
  primer LPXB reverse 1149
ggggcctcgataccgacgaagacatcagg

SEQ ID NO: 1160 - DNA, 29 bp, Artificial
  primer LPXB reverse 1150
ggggcctcgataccgacgaagacatccgg

SEQ ID NO: 1161 - DNA, 29 bp, Artificial
  primer LPXB reverse 1151
ggggcctcgataccgacgaagacatcggg

SEQ ID NO: 1162 - DNA, 29 bp, Artificial
  primer LPXB reverse 1152
ggggcctcgataccgacgaagacatctgg

SEQ ID NO: 1163 - DNA, 29 bp, Artificial
  primer LPXB reverse 1153
ggggcctcgatcccaacaaaaacatcagg

SEQ ID NO: 1164 - DNA, 29 bp, Artificial
  primer LPXB reverse 1154
ggggcctcgatcccaacaaaaacatccgg

SEQ ID NO: 1165 - DNA, 29 bp, Artificial
  primer LPXB reverse 1155
ggggcctcgatcccaacaaaaacatcggg

SEQ ID NO: 1166 - DNA, 29 bp, Artificial
  primer LPXB reverse 1156
ggggcctcgatcccaacaaaaacatctgg

SEQ ID NO: 1167 - DNA, 29 bp, Artificial
  primer LPXB reverse 1157
ggggcctcgatcccaacaaacacatcagg

SEQ ID NO: 1168 - DNA, 29 bp, Artificial
  primer LPXB reverse 1158
ggggcctcgatcccaacaaacacatccgg

SEQ ID NO: 1169 - DNA, 29 bp, Artificial
  primer LPXB reverse 1159
ggggcctcgatcccaacaaacacatctgg

SEQ ID NO: 1170 - DNA, 29 bp, Artificial
  primer LPXB reverse 1160
ggggcctcgatcccaacaaacacatcggg

SEQ ID NO: 1171 - DNA, 29 bp, Artificial
  primer LPXB reverse 1161
ggggcctcgatcccaacaaagacatcagg

SEQ ID NO: 1172 - DNA, 29 bp, Artificial
  primer LPXB reverse 1162
ggggcctcgatcccaacaaagacatccgg

SEQ ID NO: 1173 - DNA, 29 bp, Artificial
  primer LPXB reverse 1163
ggggcctcgatcccaacaaagacatcggg

SEQ ID NO: 1174 - DNA, 29 bp, Artificial
  primer LPXB reverse 1164
ggggcctcgatcccaacaaagacatctgg

SEQ ID NO: 1175 - DNA, 29 bp, Artificial
  primer LPXB reverse 1165
ggggcctcgatcccaacgaaaacatcagg

SEQ ID NO: 1176 - DNA, 29 bp, Artificial
  primer LPXB reverse 1166
ggggcctcgatcccaacgaaaacatccgg

SEQ ID NO: 1177 - DNA, 29 bp, Artificial
  primer LPXB reverse 1167
ggggcctcgatcccaacgaaaacatcggg

SEQ ID NO: 1178 - DNA, 29 bp, Artificial
  primer LPXB reverse 1168
ggggcctcgatcccaacgaaaacatctgg

SEQ ID NO: 1179 - DNA, 29 bp, Artificial
  primer LPXB reverse 1169
ggggcctcgatcccaacgaacacatcagg

SEQ ID NO: 1180 - DNA, 29 bp, Artificial
  primer LPXB reverse 1170
ggggcctcgatcccaacgaacacatccgg

SEQ ID NO: 1181 - DNA, 29 bp, Artificial
  primer LPXB reverse 1171
ggggcctcgatcccaacgaacacatctgg

SEQ ID NO: 1182 - DNA, 29 bp, Artificial
  primer LPXB reverse 1172
ggggcctcgatcccaacgaacacatcggg

SEQ ID NO: 1183 - DNA, 29 bp, Artificial
  primer LPXB reverse 1173
ggggcctcgatcccaacgaagacatcagg

SEQ ID NO: 1184 - DNA, 29 bp, Artificial
  primer LPXB reverse 1174
ggggcctcgatcccaacgaagacatccgg

SEQ ID NO: 1185 - DNA, 29 bp, Artificial
  primer LPXB reverse 1175
ggggcctcgatcccaacgaagacatcggg

SEQ ID NO: 1186 - DNA, 29 bp, Artificial
  primer LPXB reverse 1176
ggggcctcgatcccaacgaagacatctgg

SEQ ID NO: 1187 - DNA, 29 bp, Artificial
  primer LPXB reverse 1177
ggggcctcgatccccacaaaaacatcagg

SEQ ID NO: 1188 - DNA, 29 bp, Artificial
  primer LPXB reverse 1178
ggggcctcgatccccacaaaaacatccgg

SEQ ID NO: 1189 - DNA, 29 bp, Artificial
  primer LPXB reverse 1179
ggggcctcgatccccacaaaaacatcggg

SEQ ID NO: 1190 - DNA, 29 bp, Artificial
  primer LPXB reverse 1180
ggggcctcgatccccacaaaaacatctgg

SEQ ID NO: 1191 - DNA, 29 bp, Artificial
  primer LPXB reverse 1181
ggggcctcgatccccacaaacacatcagg

SEQ ID NO: 1192 - DNA, 29 bp, Artificial
  primer LPXB reverse 1182
ggggcctcgatccccacaaacacatccgg

SEQ ID NO: 1193 - DNA, 29 bp, Artificial
  primer LPXB reverse 1183
ggggcctcgatccccacaaacacatctgg

SEQ ID NO: 1194 - DNA, 29 bp, Artificial
  primer LPXB reverse 1184
ggggcctcgatccccacaaacacatcggg

SEQ ID NO: 1195 - DNA, 29 bp, Artificial
  primer LPXB reverse 1185
ggggcctcgatccccacaaagacatcagg

SEQ ID NO: 1196 - DNA, 29 bp, Artificial
  primer LPXB reverse 1186
ggggcctcgatccccacaaagacatccgg

SEQ ID NO: 1197 - DNA, 29 bp, Artificial
  primer LPXB reverse 1187
ggggcctcgatccccacaaagacatcggg

SEQ ID NO: 1198 - DNA, 29 bp, Artificial
  primer LPXB reverse 1188
ggggcctcgatccccacaaagacatctgg

SEQ ID NO: 1199 - DNA, 29 bp, Artificial
  primer LPXB reverse 1189
ggggcctcgatccccacgaaaacatcagg

SEQ ID NO: 1200 - DNA, 29 bp, Artificial
  primer LPXB reverse 1190
ggggcctcgatccccacgaaaacatccgg

SEQ ID NO: 1201 - DNA, 29 bp, Artificial
  primer LPXB reverse 1191
ggggcctcgatccccacgaaaacatcggg

SEQ ID NO: 1202 - DNA, 29 bp, Artificial
  primer LPXB reverse 1192
ggggcctcgatccccacgaaaacatctgg

SEQ ID NO: 1203 - DNA, 29 bp, Artificial
  primer LPXB reverse 1193
ggggcctcgatccccacgaacacatcagg

SEQ ID NO: 1204 - DNA, 29 bp, Artificial
  primer LPXB reverse 1194
ggggcctcgatccccacgaacacatccgg

SEQ ID NO: 1205 - DNA, 29 bp, Artificial
  primer LPXB reverse 1195
ggggcctcgatccccacgaacacatctgg

SEQ ID NO: 1206 - DNA, 29 bp, Artificial
  primer LPXB reverse 1196
ggggcctcgatccccacgaacacatcggg

SEQ ID NO: 1207 - DNA, 29 bp, Artificial
  primer LPXB reverse 1197
ggggcctcgatccccacgaagacatcagg

SEQ ID NO: 1208 - DNA, 29 bp, Artificial
  primer LPXB reverse 1198
ggggcctcgatccccacgaagacatccgg

SEQ ID NO: 1209 - DNA, 29 bp, Artificial
  primer LPXB reverse 1199
ggggcctcgatccccacgaagacatcggg

SEQ ID NO: 1210 - DNA, 29 bp, Artificial
  primer LPXB reverse 1200
ggggcctcgatccccacgaagacatctgg

SEQ ID NO: 1211 - DNA, 29 bp, Artificial
  primer LPXB reverse 1201
ggggcctcgatcccgacaaaaacatcagg

SEQ ID NO: 1212 - DNA, 29 bp, Artificial
  primer LPXB reverse 1202
ggggcctcgatcccgacaaaaacatccgg

SEQ ID NO: 1213 - DNA, 29 bp, Artificial
  primer LPXB reverse 1203
ggggcctcgatcccgacaaaaacatcggg

SEQ ID NO: 1214 - DNA, 29 bp, Artificial
  primer LPXB reverse 1204
ggggcctcgatcccgacaaaaacatctgg

SEQ ID NO: 1215 - DNA, 29 bp, Artificial
  primer LPXB reverse 1205
ggggcctcgatcccgacaaacacatcagg

SEQ ID NO: 1216 - DNA, 29 bp, Artificial
  primer LPXB reverse 1206
ggggcctcgatcccgacaaacacatccgg

SEQ ID NO: 1217 - DNA, 29 bp, Artificial
  primer LPXB reverse 1207
ggggcctcgatcccgacaaacacatctgg

SEQ ID NO: 1218 - DNA, 29 bp, Artificial
  primer LPXB reverse 1208
ggggcctcgatcccgacaaacacatcggg

SEQ ID NO: 1219 - DNA, 29 bp, Artificial
  primer LPXB reverse 1209
ggggcctcgatcccgacaaagacatcagg

SEQ ID NO: 1220 - DNA, 29 bp, Artificial
  primer LPXB reverse 1210
ggggcctcgatcccgacaaagacatccgg

SEQ ID NO: 1221 - DNA, 29 bp, Artificial
  primer LPXB reverse 1211
ggggcctcgatcccgacaaagacatcggg

SEQ ID NO: 1222 - DNA, 29 bp, Artificial
  primer LPXB reverse 1212
ggggcctcgatcccgacaaagacatctgg

SEQ ID NO: 1223 - DNA, 29 bp, Artificial
  primer LPXB reverse 1213
ggggcctcgatcccgacgaaaacatcagg

SEQ ID NO: 1224 - DNA, 29 bp, Artificial
  primer LPXB reverse 1214
ggggcctcgatcccgacgaaaacatccgg

SEQ ID NO: 1225 - DNA, 29 bp, Artificial
  primer LPXB reverse 1215
ggggcctcgatcccgacgaaaacatcggg

SEQ ID NO: 1226 - DNA, 29 bp, Artificial
  primer LPXB reverse 1216
ggggcctcgatcccgacgaaaacatctgg

SEQ ID NO: 1227 - DNA, 29 bp, Artificial
  primer LPXB reverse 1217
ggggcctcgatcccgacgaacacatcagg

SEQ ID NO: 1228 - DNA, 29 bp, Artificial
  primer LPXB reverse 1218
ggggcctcgatcccgacgaacacatccgg

SEQ ID NO: 1229 - DNA, 29 bp, Artificial
  primer LPXB reverse 1219
ggggcctcgatcccgacgaacacatctgg

SEQ ID NO: 1230 - DNA, 29 bp, Artificial
  primer LPXB reverse 1220
ggggcctcgatcccgacgaacacatcggg

SEQ ID NO: 1231 - DNA, 29 bp, Artificial
  primer LPXB reverse 1221
ggggcctcgatcccgacgaagacatcagg

SEQ ID NO: 1232 - DNA, 29 bp, Artificial
  primer LPXB reverse 1222
ggggcctcgatcccgacgaagacatccgg

SEQ ID NO: 1233 - DNA, 29 bp, Artificial
  primer LPXB reverse 1223
ggggcctcgatcccgacgaagacatcggg

SEQ ID NO: 1234 - DNA, 29 bp, Artificial
  primer LPXB reverse 1224
ggggcctcgatcccgacgaagacatctgg

SEQ ID NO: 1235 - DNA, 29 bp, Artificial
  primer LPXB reverse 1225
ggggcctcgattccaacaaaaacatcagg

SEQ ID NO: 1236 - DNA, 29 bp, Artificial
  primer LPXB reverse 1226
ggggcctcgattccaacaaaaacatccgg

SEQ ID NO: 1237 - DNA, 29 bp, Artificial
  primer LPXB reverse 1227
ggggcctcgattccaacaaaaacatcggg

SEQ ID NO: 1238 - DNA, 29 bp, Artificial
  primer LPXB reverse 1228
ggggcctcgattccaacaaaaacatctgg

SEQ ID NO: 1239 - DNA, 29 bp, Artificial
  primer LPXB reverse 1229
ggggcctcgattccaacaaacacatcagg

SEQ ID NO: 1240 - DNA, 29 bp, Artificial
  primer LPXB reverse 1230
ggggcctcgattccaacaaacacatccgg

SEQ ID NO: 1241 - DNA, 29 bp, Artificial
  primer LPXB reverse 1231
ggggcctcgattccaacaaacacatctgg

SEQ ID NO: 1242 - DNA, 29 bp, Artificial
  primer LPXB reverse 1232
ggggcctcgattccaacaaacacatcggg

SEQ ID NO: 1243 - DNA, 29 bp, Artificial
  primer LPXB reverse 1233
ggggcctcgattccaacaaagacatcagg

SEQ ID NO: 1244 - DNA, 29 bp, Artificial
  primer LPXB reverse 1234
ggggcctcgattccaacaaagacatccgg

SEQ ID NO: 1245 - DNA, 29 bp, Artificial
  primer LPXB reverse 1235
ggggcctcgattccaacaaagacatcggg

SEQ ID NO: 1246 - DNA, 29 bp, Artificial
  primer LPXB reverse 1236
ggggcctcgattccaacaaagacatctgg

SEQ ID NO: 1247 - DNA, 29 bp, Artificial
  primer LPXB reverse 1237
ggggcctcgattccaacgaaaacatcagg

SEQ ID NO: 1248 - DNA, 29 bp, Artificial
  primer LPXB reverse 1238
ggggcctcgattccaacgaaaacatccgg

SEQ ID NO: 1249 - DNA, 29 bp, Artificial
  primer LPXB reverse 1239
ggggcctcgattccaacgaaaacatcggg

SEQ ID NO: 1250 - DNA, 29 bp, Artificial
  primer LPXB reverse 1240
ggggcctcgattccaacgaaaacatctgg

SEQ ID NO: 1251 - DNA, 29 bp, Artificial
  primer LPXB reverse 1241
ggggcctcgattccaacgaacacatcagg

SEQ ID NO: 1252 - DNA, 29 bp, Artificial
  primer LPXB reverse 1242
ggggcctcgattccaacgaacacatccgg

SEQ ID NO: 1253 - DNA, 29 bp, Artificial
  primer LPXB reverse 1243
ggggcctcgattccaacgaacacatctgg

SEQ ID NO: 1254 - DNA, 29 bp, Artificial
  primer LPXB reverse 1244
ggggcctcgattccaacgaacacatcggg

SEQ ID NO: 1255 - DNA, 29 bp, Artificial
  primer LPXB reverse 1245
ggggcctcgattccaacgaagacatcagg

SEQ ID NO: 1256 - DNA, 29 bp, Artificial
  primer LPXB reverse 1246
ggggcctcgattccaacgaagacatccgg

SEQ ID NO: 1257 - DNA, 29 bp, Artificial
  primer LPXB reverse 1247
ggggcctcgattccaacgaagacatcggg

SEQ ID NO: 1258 - DNA, 29 bp, Artificial
  primer LPXB reverse 1248
ggggcctcgattccaacgaagacatctgg

SEQ ID NO: 1259 - DNA, 29 bp, Artificial
  primer LPXB reverse 1249
ggggcctcgattcccacaaaaacatcagg

SEQ ID NO: 1260 - DNA, 29 bp, Artificial
  primer LPXB reverse 1250
ggggcctcgattcccacaaaaacatccgg

SEQ ID NO: 1261 - DNA, 29 bp, Artificial
  primer LPXB reverse 1251
ggggcctcgattcccacaaaaacatcggg

SEQ ID NO: 1262 - DNA, 29 bp, Artificial
  primer LPXB reverse 1252
ggggcctcgattcccacaaaaacatctgg

SEQ ID NO: 1263 - DNA, 29 bp, Artificial
  primer LPXB reverse 1253
ggggcctcgattcccacaaacacatcagg

SEQ ID NO: 1264 - DNA, 29 bp, Artificial
  primer LPXB reverse 1254
ggggcctcgattcccacaaacacatccgg

SEQ ID NO: 1265 - DNA, 29 bp, Artificial
  primer LPXB reverse 1255
ggggcctcgattcccacaaacacatctgg

SEQ ID NO: 1266 - DNA, 29 bp, Artificial
  primer LPXB reverse 1256
ggggcctcgattcccacaaacacatcggg

SEQ ID NO: 1267 - DNA, 29 bp, Artificial
  primer LPXB reverse 1257
ggggcctcgattcccacaaagacatcagg

SEQ ID NO: 1268 - DNA, 29 bp, Artificial
  primer LPXB reverse 1258
ggggcctcgattcccacaaagacatccgg

SEQ ID NO: 1269 - DNA, 29 bp, Artificial
  primer LPXB reverse 1259
ggggcctcgattcccacaaagacatcggg

SEQ ID NO: 1270 - DNA, 29 bp, Artificial
  primer LPXB reverse 1260
ggggcctcgattcccacaaagacatctgg

SEQ ID NO: 1271 - DNA, 29 bp, Artificial
  primer LPXB reverse 1261
ggggcctcgattcccacgaaaacatcagg

SEQ ID NO: 1272 - DNA, 29 bp, Artificial
  primer LPXB reverse 1262
ggggcctcgattcccacgaaaacatccgg

SEQ ID NO: 1273 - DNA, 29 bp, Artificial
  primer LPXB reverse 1263
ggggcctcgattcccacgaaaacatcggg

SEQ ID NO: 1274 - DNA, 29 bp, Artificial
  primer LPXB reverse 1264
ggggcctcgattcccacgaaaacatctgg

SEQ ID NO: 1275 - DNA, 29 bp, Artificial
  primer LPXB reverse 1265
ggggcctcgattcccacgaacacatcagg

SEQ ID NO: 1276 - DNA, 29 bp, Artificial
  primer LPXB reverse 1266
ggggcctcgattcccacgaacacatccgg

SEQ ID NO: 1277 - DNA, 29 bp, Artificial
  primer LPXB reverse 1267
ggggcctcgattcccacgaacacatctgg

SEQ ID NO: 1278 - DNA, 29 bp, Artificial
  primer LPXB reverse 1268
ggggcctcgattcccacgaacacatcggg

SEQ ID NO: 1279 - DNA, 29 bp, Artificial
  primer LPXB reverse 1269
ggggcctcgattcccacgaagacatcagg

SEQ ID NO: 1280 - DNA, 29 bp, Artificial
  primer LPXB reverse 1270
ggggcctcgattcccacgaagacatccgg

SEQ ID NO: 1281 - DNA, 29 bp, Artificial
  primer LPXB reverse 1271
ggggcctcgattcccacgaagacatcggg

SEQ ID NO: 1282 - DNA, 29 bp, Artificial
  primer LPXB reverse 1272
ggggcctcgattcccacgaagacatctgg

SEQ ID NO: 1283 - DNA, 29 bp, Artificial
  primer LPXB reverse 1273
ggggcctcgattccgacaaaaacatcagg

SEQ ID NO: 1284 - DNA, 29 bp, Artificial
  primer LPXB reverse 1274
ggggcctcgattccgacaaaaacatccgg

SEQ ID NO: 1285 - DNA, 29 bp, Artificial
  primer LPXB reverse 1275
ggggcctcgattccgacaaaaacatcggg

SEQ ID NO: 1286 - DNA, 29 bp, Artificial
  primer LPXB reverse 1276
ggggcctcgattccgacaaaaacatctgg

SEQ ID NO: 1287 - DNA, 29 bp, Artificial
  primer LPXB reverse 1277
ggggcctcgattccgacaaacacatcagg

SEQ ID NO: 1288 - DNA, 29 bp, Artificial
  primer LPXB reverse 1278
ggggcctcgattccgacaaacacatccgg

SEQ ID NO: 1289 - DNA, 29 bp, Artificial
  primer LPXB reverse 1279
ggggcctcgattccgacaaacacatctgg

SEQ ID NO: 1290 - DNA, 29 bp, Artificial
  primer LPXB reverse 1280
ggggcctcgattccgacaaacacatcggg

SEQ ID NO: 1291 - DNA, 29 bp, Artificial
  primer LPXB reverse 1281
ggggcctcgattccgacaaagacatcagg

SEQ ID NO: 1292 - DNA, 29 bp, Artificial
  primer LPXB reverse 1282
ggggcctcgattccgacaaagacatccgg

SEQ ID NO: 1293 - DNA, 29 bp, Artificial
  primer LPXB reverse 1283
ggggcctcgattccgacaaagacatcggg

SEQ ID NO: 1294 - DNA, 29 bp, Artificial
  primer LPXB reverse 1284
ggggcctcgattccgacaaagacatctgg

SEQ ID NO: 1295 - DNA, 29 bp, Artificial
  primer LPXB reverse 1285
ggggcctcgattccgacgaaaacatcagg

SEQ ID NO: 1296 - DNA, 29 bp, Artificial
  primer LPXB reverse 1286
ggggcctcgattccgacgaaaacatccgg

SEQ ID NO: 1297 - DNA, 29 bp, Artificial
  primer LPXB reverse 1287
ggggcctcgattccgacgaaaacatcggg

SEQ ID NO: 1298 - DNA, 29 bp, Artificial
  primer LPXB reverse 1288
ggggcctcgattccgacgaaaacatctgg

SEQ ID NO: 1299 - DNA, 29 bp, Artificial
  primer LPXB reverse 1289
ggggcctcgattccgacgaacacatcagg

SEQ ID NO: 1300 - DNA, 29 bp, Artificial
  primer LPXB reverse 1290
ggggcctcgattccgacgaacacatccgg

SEQ ID NO: 1301 - DNA, 29 bp, Artificial
  primer LPXB reverse 1291
ggggcctcgattccgacgaacacatctgg

SEQ ID NO: 1302 - DNA, 29 bp, Artificial
  primer LPXB reverse 1292
ggggcctcgattccgacgaacacatcggg

SEQ ID NO: 1303 - DNA, 29 bp, Artificial
  primer LPXB reverse 1293
ggggcctcgattccgacgaagacatcagg

SEQ ID NO: 1304 - DNA, 29 bp, Artificial
  primer LPXB reverse 1294
ggggcctcgattccgacgaagacatccgg

SEQ ID NO: 1305 - DNA, 29 bp, Artificial
  primer LPXB reverse 1295
ggggcctcgattccgacgaagacatcggg

SEQ ID NO: 1306 - DNA, 29 bp, Artificial
  primer LPXB reverse 1296
ggggcctcgattccgacgaagacatctgg

SEQ ID NO: 1307 - DNA, 29 bp, Artificial
  primer LPXB reverse 1297
ggggcttcaataccaacaaaaacatcagg

SEQ ID NO: 1308 - DNA, 29 bp, Artificial
  primer LPXB reverse 1298
ggggcttcaataccaacaaaaacatccgg

SEQ ID NO: 1309 - DNA, 29 bp, Artificial
  primer LPXB reverse 1299
ggggcttcaataccaacaaaaacatcggg

SEQ ID NO: 1310 - DNA, 29 bp, Artificial
  primer LPXB reverse 1300
ggggcttcaataccaacaaaaacatctgg

SEQ ID NO: 1311 - DNA, 29 bp, Artificial
  primer LPXB reverse 1301
ggggcttcaataccaacaaacacatcagg

SEQ ID NO: 1312 - DNA, 29 bp, Artificial
  primer LPXB reverse 1302
ggggcttcaataccaacaaacacatccgg

SEQ ID NO: 1313 - DNA, 29 bp, Artificial
  primer LPXB reverse 1303
ggggcttcaataccaacaaacacatctgg

SEQ ID NO: 1314 - DNA, 29 bp, Artificial
  primer LPXB reverse 1304
ggggcttcaataccaacaaacacatcggg

SEQ ID NO: 1315 - DNA, 29 bp, Artificial
  primer LPXB reverse 1305
ggggcttcaataccaacaaagacatcagg

SEQ ID NO: 1316 - DNA, 29 bp, Artificial
  primer LPXB reverse 1306
ggggcttcaataccaacaaagacatccgg

SEQ ID NO: 1317 - DNA, 29 bp, Artificial
  primer LPXB reverse 1307
ggggcttcaataccaacaaagacatcggg

SEQ ID NO: 1318 - DNA, 29 bp, Artificial
  primer LPXB reverse 1308
ggggcttcaataccaacaaagacatctgg

SEQ ID NO: 1319 - DNA, 29 bp, Artificial
  primer LPXB reverse 1309
ggggcttcaataccaacgaaaacatcagg

SEQ ID NO: 1320 - DNA, 29 bp, Artificial
  primer LPXB reverse 1310
ggggcttcaataccaacgaaaacatccgg

SEQ ID NO: 1321 - DNA, 29 bp, Artificial
  primer LPXB reverse 1311
ggggcttcaataccaacgaaaacatcggg

SEQ ID NO: 1322 - DNA, 29 bp, Artificial
  primer LPXB reverse 1312
ggggcttcaataccaacgaaaacatctgg

SEQ ID NO: 1323 - DNA, 29 bp, Artificial
  primer LPXB reverse 1313
ggggcttcaataccaacgaacacatcagg

SEQ ID NO: 1324 - DNA, 29 bp, Artificial
  primer LPXB reverse 1314
ggggcttcaataccaacgaacacatccgg

SEQ ID NO: 1325 - DNA, 29 bp, Artificial
  primer LPXB reverse 1315
ggggcttcaataccaacgaacacatctgg

SEQ ID NO: 1326 - DNA, 29 bp, Artificial
  primer LPXB reverse 1316
ggggcttcaataccaacgaacacatcggg

SEQ ID NO: 1327 - DNA, 29 bp, Artificial
  primer LPXB reverse 1317
ggggcttcaataccaacgaagacatcagg

SEQ ID NO: 1328 - DNA, 29 bp, Artificial
  primer LPXB reverse 1318
ggggcttcaataccaacgaagacatccgg

SEQ ID NO: 1329 - DNA, 29 bp, Artificial
  primer LPXB reverse 1319
ggggcttcaataccaacgaagacatcggg

SEQ ID NO: 1330 - DNA, 29 bp, Artificial
  primer LPXB reverse 1320
ggggcttcaataccaacgaagacatctgg

SEQ ID NO: 1331 - DNA, 29 bp, Artificial
  primer LPXB reverse 1321
ggggcttcaatacccacaaaaacatcagg

SEQ ID NO: 1332 - DNA, 29 bp, Artificial
  primer LPXB reverse 1322
ggggcttcaatacccacaaaaacatccgg

SEQ ID NO: 1333 - DNA, 29 bp, Artificial
  primer LPXB reverse 1323
ggggcttcaatacccacaaaaacatcggg

SEQ ID NO: 1334 - DNA, 29 bp, Artificial
  primer LPXB reverse 1324
ggggcttcaatacccacaaaaacatctgg

SEQ ID NO: 1335 - DNA, 29 bp, Artificial
  primer LPXB reverse 1325
ggggcttcaatacccacaaacacatcagg

SEQ ID NO: 1336 - DNA, 29 bp, Artificial
  primer LPXB reverse 1326
ggggcttcaatacccacaaacacatccgg

SEQ ID NO: 1337 - DNA, 29 bp, Artificial
  primer LPXB reverse 1327
ggggcttcaatacccacaaacacatctgg

SEQ ID NO: 1338 - DNA, 29 bp, Artificial
  primer LPXB reverse 1328
ggggcttcaatacccacaaacacatcggg

SEQ ID NO: 1339 - DNA, 29 bp, Artificial
  primer LPXB reverse 1329
ggggcttcaatacccacaaagacatcagg

SEQ ID NO: 1340 - DNA, 29 bp, Artificial
  primer LPXB reverse 1330
ggggcttcaatacccacaaagacatccgg

SEQ ID NO: 1341 - DNA, 29 bp, Artificial
  primer LPXB reverse 1331
ggggcttcaatacccacaaagacatcggg

SEQ ID NO: 1342 - DNA, 29 bp, Artificial
  primer LPXB reverse 1332
ggggcttcaatacccacaaagacatctgg

SEQ ID NO: 1343 - DNA, 29 bp, Artificial
  primer LPXB reverse 1333
ggggcttcaatacccacgaaaacatcagg

SEQ ID NO: 1344 - DNA, 29 bp, Artificial
  primer LPXB reverse 1334
ggggcttcaatacccacgaaaacatccgg

SEQ ID NO: 1345 - DNA, 29 bp, Artificial
  primer LPXB reverse 1335
ggggcttcaatacccacgaaaacatcggg

SEQ ID NO: 1346 - DNA, 29 bp, Artificial
  primer LPXB reverse 1336
ggggcttcaatacccacgaaaacatctgg

SEQ ID NO: 1347 - DNA, 29 bp, Artificial
  primer LPXB reverse 1337
ggggcttcaatacccacgaacacatcagg

SEQ ID NO: 1348 - DNA, 29 bp, Artificial
  primer LPXB reverse 1338
ggggcttcaatacccacgaacacatccgg

SEQ ID NO: 1349 - DNA, 29 bp, Artificial
  primer LPXB reverse 1339
ggggcttcaatacccacgaacacatctgg

SEQ ID NO: 1350 - DNA, 29 bp, Artificial
  primer LPXB reverse 1340
ggggcttcaatacccacgaacacatcggg

SEQ ID NO: 1351 - DNA, 29 bp, Artificial
  primer LPXB reverse 1341
ggggcttcaatacccacgaagacatcagg

SEQ ID NO: 1352 - DNA, 29 bp, Artificial
  primer LPXB reverse 1342
ggggcttcaatacccacgaagacatccgg

SEQ ID NO: 1353 - DNA, 29 bp, Artificial
  primer LPXB reverse 1343
ggggcttcaatacccacgaagacatcggg

SEQ ID NO: 1354 - DNA, 29 bp, Artificial
  primer LPXB reverse 1344
ggggcttcaatacccacgaagacatctgg

SEQ ID NO: 1355 - DNA, 29 bp, Artificial
  primer LPXB reverse 1345
ggggcttcaataccgacaaaaacatcagg

SEQ ID NO: 1356 - DNA, 29 bp, Artificial
  primer LPXB reverse 1346
ggggcttcaataccgacaaaaacatccgg

SEQ ID NO: 1357 - DNA, 29 bp, Artificial
  primer LPXB reverse 1347
ggggcttcaataccgacaaaaacatcggg

SEQ ID NO: 1358 - DNA, 29 bp, Artificial
  primer LPXB reverse 1348
ggggcttcaataccgacaaaaacatctgg

SEQ ID NO: 1359 - DNA, 29 bp, Artificial
  primer LPXB reverse 1349
ggggcttcaataccgacaaacacatcagg

SEQ ID NO: 1360 - DNA, 29 bp, Artificial
  primer LPXB reverse 1350
ggggcttcaataccgacaaacacatccgg

SEQ ID NO: 1361 - DNA, 29 bp, Artificial
  primer LPXB reverse 1351
ggggcttcaataccgacaaacacatctgg

SEQ ID NO: 1362 - DNA, 29 bp, Artificial
  primer LPXB reverse 1352
ggggcttcaataccgacaaacacatcggg

SEQ ID NO: 1363 - DNA, 29 bp, Artificial
  primer LPXB reverse 1353
ggggcttcaataccgacaaagacatcagg

SEQ ID NO: 1364 - DNA, 29 bp, Artificial
  primer LPXB reverse 1354
ggggcttcaataccgacaaagacatccgg

SEQ ID NO: 1365 - DNA, 29 bp, Artificial
  primer LPXB reverse 1355
ggggcttcaataccgacaaagacatcggg

SEQ ID NO: 1366 - DNA, 29 bp, Artificial
  primer LPXB reverse 1356
ggggcttcaataccgacaaagacatctgg

SEQ ID NO: 1367 - DNA, 29 bp, Artificial
  primer LPXB reverse 1357
ggggcttcaataccgacgaaaacatcagg

SEQ ID NO: 1368 - DNA, 29 bp, Artificial
  primer LPXB reverse 1358
ggggcttcaataccgacgaaaacatccgg

SEQ ID NO: 1369 - DNA, 29 bp, Artificial
  primer LPXB reverse 1359
ggggcttcaataccgacgaaaacatcggg

SEQ ID NO: 1370 - DNA, 29 bp, Artificial
  primer LPXB reverse 1360
ggggcttcaataccgacgaaaacatctgg

SEQ ID NO: 1371 - DNA, 29 bp, Artificial
  primer LPXB reverse 1361
ggggcttcaataccgacgaacacatcagg

SEQ ID NO: 1372 - DNA, 29 bp, Artificial
  primer LPXB reverse 1362
ggggcttcaataccgacgaacacatccgg

SEQ ID NO: 1373 - DNA, 29 bp, Artificial
  primer LPXB reverse 1363
ggggcttcaataccgacgaacacatctgg

SEQ ID NO: 1374 - DNA, 29 bp, Artificial
  primer LPXB reverse 1364
ggggcttcaataccgacgaacacatcggg

SEQ ID NO: 1375 - DNA, 29 bp, Artificial
  primer LPXB reverse 1365
ggggcttcaataccgacgaagacatcagg

SEQ ID NO: 1376 - DNA, 29 bp, Artificial
  primer LPXB reverse 1366
ggggcttcaataccgacgaagacatccgg

SEQ ID NO: 1377 - DNA, 29 bp, Artificial
  primer LPXB reverse 1367
ggggcttcaataccgacgaagacatcggg

SEQ ID NO: 1378 - DNA, 29 bp, Artificial
  primer LPXB reverse 1368
ggggcttcaataccgacgaagacatctgg

SEQ ID NO: 1379 - DNA, 29 bp, Artificial
  primer LPXB reverse 1369
ggggcttcaatcccaacaaaaacatcagg

SEQ ID NO: 1380 - DNA, 29 bp, Artificial
  primer LPXB reverse 1370
ggggcttcaatcccaacaaaaacatccgg

SEQ ID NO: 1381 - DNA, 29 bp, Artificial
  primer LPXB reverse 1371
ggggcttcaatcccaacaaaaacatcggg

SEQ ID NO: 1382 - DNA, 29 bp, Artificial
  primer LPXB reverse 1372
ggggcttcaatcccaacaaaaacatctgg

SEQ ID NO: 1383 - DNA, 29 bp, Artificial
  primer LPXB reverse 1373
ggggcttcaatcccaacaaacacatcagg

SEQ ID NO: 1384 - DNA, 29 bp, Artificial
  primer LPXB reverse 1374
ggggcttcaatcccaacaaacacatccgg

SEQ ID NO: 1385 - DNA, 29 bp, Artificial
  primer LPXB reverse 1375
ggggcttcaatcccaacaaacacatctgg

SEQ ID NO: 1386 - DNA, 29 bp, Artificial
  primer LPXB reverse 1376
ggggcttcaatcccaacaaacacatcggg

SEQ ID NO: 1387 - DNA, 29 bp, Artificial
  primer LPXB reverse 1377
ggggcttcaatcccaacaaagacatcagg

SEQ ID NO: 1388 - DNA, 29 bp, Artificial
  primer LPXB reverse 1378
ggggcttcaatcccaacaaagacatccgg

SEQ ID NO: 1389 - DNA, 29 bp, Artificial
  primer LPXB reverse 1379
ggggcttcaatcccaacaaagacatcggg

SEQ ID NO: 1390 - DNA, 29 bp, Artificial
  primer LPXB reverse 1380
ggggcttcaatcccaacaaagacatctgg

SEQ ID NO: 1391 - DNA, 29 bp, Artificial
  primer LPXB reverse 1381
ggggcttcaatcccaacgaaaacatcagg

SEQ ID NO: 1392 - DNA, 29 bp, Artificial
  primer LPXB reverse 1382
ggggcttcaatcccaacgaaaacatccgg

SEQ ID NO: 1393 - DNA, 29 bp, Artificial
  primer LPXB reverse 1383
ggggcttcaatcccaacgaaaacatcggg

SEQ ID NO: 1394 - DNA, 29 bp, Artificial
  primer LPXB reverse 1384
ggggcttcaatcccaacgaaaacatctgg

SEQ ID NO: 1395 - DNA, 29 bp, Artificial
  primer LPXB reverse 1385
ggggcttcaatcccaacgaacacatcagg

SEQ ID NO: 1396 - DNA, 29 bp, Artificial
  primer LPXB reverse 1386
ggggcttcaatcccaacgaacacatccgg

SEQ ID NO: 1397 - DNA, 29 bp, Artificial
  primer LPXB reverse 1387
ggggcttcaatcccaacgaacacatctgg

SEQ ID NO: 1398 - DNA, 29 bp, Artificial
  primer LPXB reverse 1388
ggggcttcaatcccaacgaacacatcggg

SEQ ID NO: 1399 - DNA, 29 bp, Artificial
  primer LPXB reverse 1389
ggggcttcaatcccaacgaagacatcagg

SEQ ID NO: 1400 - DNA, 29 bp, Artificial
  primer LPXB reverse 1390
ggggcttcaatcccaacgaagacatccgg

SEQ ID NO: 1401 - DNA, 29 bp, Artificial
  primer LPXB reverse 1391
ggggcttcaatcccaacgaagacatcggg

SEQ ID NO: 1402 - DNA, 29 bp, Artificial
  primer LPXB reverse 1392
ggggcttcaatcccaacgaagacatctgg

SEQ ID NO: 1403 - DNA, 29 bp, Artificial
  primer LPXB reverse 1393
ggggcttcaatccccacaaaaacatcagg

SEQ ID NO: 1404 - DNA, 29 bp, Artificial
  primer LPXB reverse 1394
ggggcttcaatccccacaaaaacatccgg

SEQ ID NO: 1405 - DNA, 29 bp, Artificial
  primer LPXB reverse 1395
ggggcttcaatccccacaaaaacatcggg

SEQ ID NO: 1406 - DNA, 29 bp, Artificial
  primer LPXB reverse 1396
ggggcttcaatccccacaaaaacatctgg

SEQ ID NO: 1407 - DNA, 29 bp, Artificial
  primer LPXB reverse 1397
ggggcttcaatccccacaaacacatcagg

SEQ ID NO: 1408 - DNA, 29 bp, Artificial
  primer LPXB reverse 1398
ggggcttcaatccccacaaacacatccgg

SEQ ID NO: 1409 - DNA, 29 bp, Artificial
  primer LPXB reverse 1399
ggggcttcaatccccacaaacacatctgg

SEQ ID NO: 1410 - DNA, 29 bp, Artificial
  primer LPXB reverse 1400
ggggcttcaatccccacaaacacatcggg

SEQ ID NO: 1411 - DNA, 29 bp, Artificial
  primer LPXB reverse 1401
ggggcttcaatccccacaaagacatcagg

SEQ ID NO: 1412 - DNA, 29 bp, Artificial
  primer LPXB reverse 1402
ggggcttcaatccccacaaagacatccgg

SEQ ID NO: 1413 - DNA, 29 bp, Artificial
  primer LPXB reverse 1403
ggggcttcaatccccacaaagacatcggg

SEQ ID NO: 1414 - DNA, 29 bp, Artificial
  primer LPXB reverse 1404
ggggcttcaatccccacaaagacatctgg

SEQ ID NO: 1415 - DNA, 29 bp, Artificial
  primer LPXB reverse 1405
ggggcttcaatccccacgaaaacatcagg

SEQ ID NO: 1416 - DNA, 29 bp, Artificial
  primer LPXB reverse 1406
ggggcttcaatccccacgaaaacatccgg

SEQ ID NO: 1417 - DNA, 29 bp, Artificial
  primer LPXB reverse 1407
ggggcttcaatccccacgaaaacatcggg

SEQ ID NO: 1418 - DNA, 29 bp, Artificial
  primer LPXB reverse 1408
ggggcttcaatccccacgaaaacatctgg

SEQ ID NO: 1419 - DNA, 29 bp, Artificial
  primer LPXB reverse 1409
ggggcttcaatccccacgaacacatcagg

SEQ ID NO: 1420 - DNA, 29 bp, Artificial
  primer LPXB reverse 1410
ggggcttcaatccccacgaacacatccgg

SEQ ID NO: 1421 - DNA, 29 bp, Artificial
  primer LPXB reverse 1411
ggggcttcaatccccacgaacacatctgg

SEQ ID NO: 1422 - DNA, 29 bp, Artificial
  primer LPXB reverse 1412
ggggcttcaatccccacgaacacatcggg

SEQ ID NO: 1423 - DNA, 29 bp, Artificial
  primer LPXB reverse 1413
ggggcttcaatccccacgaagacatcagg

SEQ ID NO: 1424 - DNA, 29 bp, Artificial
  primer LPXB reverse 1414
ggggcttcaatccccacgaagacatccgg

SEQ ID NO: 1425 - DNA, 29 bp, Artificial
  primer LPXB reverse 1415
ggggcttcaatccccacgaagacatcggg

SEQ ID NO: 1426 - DNA, 29 bp, Artificial
  primer LPXB reverse 1416
ggggcttcaatccccacgaagacatctgg

SEQ ID NO: 1427 - DNA, 29 bp, Artificial
  primer LPXB reverse 1417
ggggcttcaatcccgacaaaaacatcagg

SEQ ID NO: 1428 - DNA, 29 bp, Artificial
  primer LPXB reverse 1418
ggggcttcaatcccgacaaaaacatccgg

SEQ ID NO: 1429 - DNA, 29 bp, Artificial
  primer LPXB reverse 1419
ggggcttcaatcccgacaaaaacatcggg

SEQ ID NO: 1430 - DNA, 29 bp, Artificial
  primer LPXB reverse 1420
ggggcttcaatcccgacaaaaacatctgg

SEQ ID NO: 1431 - DNA, 29 bp, Artificial
  primer LPXB reverse 1421
ggggcttcaatcccgacaaacacatcagg

SEQ ID NO: 1432 - DNA, 29 bp, Artificial
  primer LPXB reverse 1422
ggggcttcaatcccgacaaacacatccgg

SEQ ID NO: 1433 - DNA, 29 bp, Artificial
  primer LPXB reverse 1423
ggggcttcaatcccgacaaacacatctgg

SEQ ID NO: 1434 - DNA, 29 bp, Artificial
  primer LPXB reverse 1424
ggggcttcaatcccgacaaacacatcggg

SEQ ID NO: 1435 - DNA, 29 bp, Artificial
  primer LPXB reverse 1425
ggggcttcaatcccgacaaagacatcagg

SEQ ID NO: 1436 - DNA, 29 bp, Artificial
  primer LPXB reverse 1426
ggggcttcaatcccgacaaagacatccgg

SEQ ID NO: 1437 - DNA, 29 bp, Artificial
  primer LPXB reverse 1427
ggggcttcaatcccgacaaagacatcggg

SEQ ID NO: 1438 - DNA, 29 bp, Artificial
  primer LPXB reverse 1428
ggggcttcaatcccgacaaagacatctgg

SEQ ID NO: 1439 - DNA, 29 bp, Artificial
  primer LPXB reverse 1429
ggggcttcaatcccgacgaaaacatcagg

SEQ ID NO: 1440 - DNA, 29 bp, Artificial
  primer LPXB reverse 1430
ggggcttcaatcccgacgaaaacatccgg

SEQ ID NO: 1441 - DNA, 29 bp, Artificial
  primer LPXB reverse 1431
ggggcttcaatcccgacgaaaacatcggg

SEQ ID NO: 1442 - DNA, 29 bp, Artificial
  primer LPXB reverse 1432
ggggcttcaatcccgacgaaaacatctgg

SEQ ID NO: 1443 - DNA, 29 bp, Artificial
  primer LPXB reverse 1433
ggggcttcaatcccgacgaacacatcagg

SEQ ID NO: 1444 - DNA, 29 bp, Artificial
  primer LPXB reverse 1434
ggggcttcaatcccgacgaacacatccgg

SEQ ID NO: 1445 - DNA, 29 bp, Artificial
  primer LPXB reverse 1435
ggggcttcaatcccgacgaacacatctgg

SEQ ID NO: 1446 - DNA, 29 bp, Artificial
  primer LPXB reverse 1436
ggggcttcaatcccgacgaacacatcggg

SEQ ID NO: 1447 - DNA, 29 bp, Artificial
  primer LPXB reverse 1437
ggggcttcaatcccgacgaagacatcagg

SEQ ID NO: 1448 - DNA, 29 bp, Artificial
  primer LPXB reverse 1438
ggggcttcaatcccgacgaagacatccgg

SEQ ID NO: 1449 - DNA, 29 bp, Artificial
  primer LPXB reverse 1439
ggggcttcaatcccgacgaagacatcggg

SEQ ID NO: 1450 - DNA, 29 bp, Artificial
  primer LPXB reverse 1440
ggggcttcaatcccgacgaagacatctgg

SEQ ID NO: 1451 - DNA, 29 bp, Artificial
  primer LPXB reverse 1441
ggggcttcaattccaacaaaaacatcagg

SEQ ID NO: 1452 - DNA, 29 bp, Artificial
  primer LPXB reverse 1442
ggggcttcaattccaacaaaaacatccgg

SEQ ID NO: 1453 - DNA, 29 bp, Artificial
  primer LPXB reverse 1443
ggggcttcaattccaacaaaaacatcggg

SEQ ID NO: 1454 - DNA, 29 bp, Artificial
  primer LPXB reverse 1444
ggggcttcaattccaacaaaaacatctgg

SEQ ID NO: 1455 - DNA, 29 bp, Artificial
  primer LPXB reverse 1445
ggggcttcaattccaacaaacacatcagg

SEQ ID NO: 1456 - DNA, 29 bp, Artificial
  primer LPXB reverse 1446
ggggcttcaattccaacaaacacatccgg

SEQ ID NO: 1457 - DNA, 29 bp, Artificial
  primer LPXB reverse 1447
ggggcttcaattccaacaaacacatctgg

SEQ ID NO: 1458 - DNA, 29 bp, Artificial
  primer LPXB reverse 1448
ggggcttcaattccaacaaacacatcggg

SEQ ID NO: 1459 - DNA, 29 bp, Artificial
  primer LPXB reverse 1449
ggggcttcaattccaacaaagacatcagg

SEQ ID NO: 1460 - DNA, 29 bp, Artificial
  primer LPXB reverse 1450
ggggcttcaattccaacaaagacatccgg

SEQ ID NO: 1461 - DNA, 29 bp, Artificial
  primer LPXB reverse 1451
ggggcttcaattccaacaaagacatcggg

SEQ ID NO: 1462 - DNA, 29 bp, Artificial
  primer LPXB reverse 1452
ggggcttcaattccaacaaagacatctgg

SEQ ID NO: 1463 - DNA, 29 bp, Artificial
  primer LPXB reverse 1453
ggggcttcaattccaacgaaaacatcagg

SEQ ID NO: 1464 - DNA, 29 bp, Artificial
  primer LPXB reverse 1454
ggggcttcaattccaacgaaaacatccgg

SEQ ID NO: 1465 - DNA, 29 bp, Artificial
  primer LPXB reverse 1455
ggggcttcaattccaacgaaaacatcggg

SEQ ID NO: 1466 - DNA, 29 bp, Artificial
  primer LPXB reverse 1456
ggggcttcaattccaacgaaaacatctgg

SEQ ID NO: 1467 - DNA, 29 bp, Artificial
  primer LPXB reverse 1457
ggggcttcaattccaacgaacacatcagg

SEQ ID NO: 1468 - DNA, 29 bp, Artificial
  primer LPXB reverse 1458
ggggcttcaattccaacgaacacatccgg

SEQ ID NO: 1469 - DNA, 29 bp, Artificial
  primer LPXB reverse 1459
ggggcttcaattccaacgaacacatctgg

SEQ ID NO: 1470 - DNA, 29 bp, Artificial
  primer LPXB reverse 1460
ggggcttcaattccaacgaacacatcggg

SEQ ID NO: 1471 - DNA, 29 bp, Artificial
  primer LPXB reverse 1461
ggggcttcaattccaacgaagacatcagg

SEQ ID NO: 1472 - DNA, 29 bp, Artificial
  primer LPXB reverse 1462
ggggcttcaattccaacgaagacatccgg

SEQ ID NO: 1473 - DNA, 29 bp, Artificial
  primer LPXB reverse 1463
ggggcttcaattccaacgaagacatcggg

SEQ ID NO: 1474 - DNA, 29 bp, Artificial
  primer LPXB reverse 1464
ggggcttcaattccaacgaagacatctgg

SEQ ID NO: 1475 - DNA, 29 bp, Artificial
  primer LPXB reverse 1465
ggggcttcaattcccacaaaaacatcagg

SEQ ID NO: 1476 - DNA, 29 bp, Artificial
  primer LPXB reverse 1466
ggggcttcaattcccacaaaaacatccgg

SEQ ID NO: 1477 - DNA, 29 bp, Artificial
  primer LPXB reverse 1467
ggggcttcaattcccacaaaaacatcggg

SEQ ID NO: 1478 - DNA, 29 bp, Artificial
  primer LPXB reverse 1468
ggggcttcaattcccacaaaaacatctgg

SEQ ID NO: 1479 - DNA, 29 bp, Artificial
  primer LPXB reverse 1469
ggggcttcaattcccacaaacacatcagg

SEQ ID NO: 1480 - DNA, 29 bp, Artificial
  primer LPXB reverse 1470
ggggcttcaattcccacaaacacatccgg

SEQ ID NO: 1481 - DNA, 29 bp, Artificial
  primer LPXB reverse 1471
ggggcttcaattcccacaaacacatctgg

SEQ ID NO: 1482 - DNA, 29 bp, Artificial
  primer LPXB reverse 1472
ggggcttcaattcccacaaacacatcggg

SEQ ID NO: 1483 - DNA, 29 bp, Artificial
  primer LPXB reverse 1473
ggggcttcaattcccacaaagacatcagg

SEQ ID NO: 1484 - DNA, 29 bp, Artificial
  primer LPXB reverse 1474
ggggcttcaattcccacaaagacatccgg

SEQ ID NO: 1485 - DNA, 29 bp, Artificial
  primer LPXB reverse 1475
ggggcttcaattcccacaaagacatcggg

SEQ ID NO: 1486 - DNA, 29 bp, Artificial
  primer LPXB reverse 1476
ggggcttcaattcccacaaagacatctgg

SEQ ID NO: 1487 - DNA, 29 bp, Artificial
  primer LPXB reverse 1477
ggggcttcaattcccacgaaaacatcagg

SEQ ID NO: 1488 - DNA, 29 bp, Artificial
  primer LPXB reverse 1478
ggggcttcaattcccacgaaaacatccgg

SEQ ID NO: 1489 - DNA, 29 bp, Artificial
  primer LPXB reverse 1479
ggggcttcaattcccacgaaaacatcggg

SEQ ID NO: 1490 - DNA, 29 bp, Artificial
  primer LPXB reverse 1480
ggggcttcaattcccacgaaaacatctgg

SEQ ID NO: 1491 - DNA, 29 bp, Artificial
  primer LPXB reverse 1481
ggggcttcaattcccacgaacacatcagg

SEQ ID NO: 1492 - DNA, 29 bp, Artificial
  primer LPXB reverse 1482
ggggcttcaattcccacgaacacatccgg

SEQ ID NO: 1493 - DNA, 29 bp, Artificial
  primer LPXB reverse 1483
ggggcttcaattcccacgaacacatctgg

SEQ ID NO: 1494 - DNA, 29 bp, Artificial
  primer LPXB reverse 1484
ggggcttcaattcccacgaacacatcggg

SEQ ID NO: 1495 - DNA, 29 bp, Artificial
  primer LPXB reverse 1485
ggggcttcaattcccacgaagacatcagg

SEQ ID NO: 1496 - DNA, 29 bp, Artificial
  primer LPXB reverse 1486
ggggcttcaattcccacgaagacatccgg

SEQ ID NO: 1497 - DNA, 29 bp, Artificial
  primer LPXB reverse 1487
ggggcttcaattcccacgaagacatcggg

SEQ ID NO: 1498 - DNA, 29 bp, Artificial
  primer LPXB reverse 1488
ggggcttcaattcccacgaagacatctgg

SEQ ID NO: 1499 - DNA, 29 bp, Artificial
  primer LPXB reverse 1489
ggggcttcaattccgacaaaaacatcagg

SEQ ID NO: 1500 - DNA, 29 bp, Artificial
  primer LPXB reverse 1490
ggggcttcaattccgacaaaaacatccgg

SEQ ID NO: 1501 - DNA, 29 bp, Artificial
  primer LPXB reverse 1491
ggggcttcaattccgacaaaaacatcggg

SEQ ID NO: 1502 - DNA, 29 bp, Artificial
  primer LPXB reverse 1492
ggggcttcaattccgacaaaaacatctgg

SEQ ID NO: 1503 - DNA, 29 bp, Artificial
  primer LPXB reverse 1493
ggggcttcaattccgacaaacacatcagg

SEQ ID NO: 1504 - DNA, 29 bp, Artificial
  primer LPXB reverse 1494
ggggcttcaattccgacaaacacatccgg

SEQ ID NO: 1505 - DNA, 29 bp, Artificial
  primer LPXB reverse 1495
ggggcttcaattccgacaaacacatctgg

SEQ ID NO: 1506 - DNA, 29 bp, Artificial
  primer LPXB reverse 1496
ggggcttcaattccgacaaacacatcggg

SEQ ID NO: 1507 - DNA, 29 bp, Artificial
  primer LPXB reverse 1497
ggggcttcaattccgacaaagacatcagg

SEQ ID NO: 1508 - DNA, 29 bp, Artificial
  primer LPXB reverse 1498
ggggcttcaattccgacaaagacatccgg

SEQ ID NO: 1509 - DNA, 29 bp, Artificial
  primer LPXB reverse 1499
ggggcttcaattccgacaaagacatcggg

SEQ ID NO: 1510 - DNA, 29 bp, Artificial
  primer LPXB reverse 1500
ggggcttcaattccgacaaagacatctgg

SEQ ID NO: 1511 - DNA, 29 bp, Artificial
  primer LPXB reverse 1501
ggggcttcaattccgacgaaaacatcagg

SEQ ID NO: 1512 - DNA, 29 bp, Artificial
  primer LPXB reverse 1502
ggggcttcaattccgacgaaaacatccgg

SEQ ID NO: 1513 - DNA, 29 bp, Artificial
  primer LPXB reverse 1503
ggggcttcaattccgacgaaaacatcggg

SEQ ID NO: 1514 - DNA, 29 bp, Artificial
  primer LPXB reverse 1504
ggggcttcaattccgacgaaaacatctgg

SEQ ID NO: 1515 - DNA, 29 bp, Artificial
  primer LPXB reverse 1505
ggggcttcaattccgacgaacacatcagg

SEQ ID NO: 1516 - DNA, 29 bp, Artificial
  primer LPXB reverse 1506
ggggcttcaattccgacgaacacatccgg

SEQ ID NO: 1517 - DNA, 29 bp, Artificial
  primer LPXB reverse 1507
ggggcttcaattccgacgaacacatctgg

SEQ ID NO: 1518 - DNA, 29 bp, Artificial
  primer LPXB reverse 1508
ggggcttcaattccgacgaacacatcggg

SEQ ID NO: 1519 - DNA, 29 bp, Artificial
  primer LPXB reverse 1509
ggggcttcaattccgacgaagacatcagg

SEQ ID NO: 1520 - DNA, 29 bp, Artificial
  primer LPXB reverse 1510
ggggcttcaattccgacgaagacatccgg

SEQ ID NO: 1521 - DNA, 29 bp, Artificial
  primer LPXB reverse 1511
ggggcttcaattccgacgaagacatcggg

SEQ ID NO: 1522 - DNA, 29 bp, Artificial
  primer LPXB reverse 1512
ggggcttcaattccgacgaagacatctgg

SEQ ID NO: 1523 - DNA, 29 bp, Artificial
  primer LPXB reverse 1513
ggggcttcgataccaacaaaaacatcagg

SEQ ID NO: 1524 - DNA, 29 bp, Artificial
  primer LPXB reverse 1514
ggggcttcgataccaacaaaaacatccgg

SEQ ID NO: 1525 - DNA, 29 bp, Artificial
  primer LPXB reverse 1515
ggggcttcgataccaacaaaaacatcggg

SEQ ID NO: 1526 - DNA, 29 bp, Artificial
  primer LPXB reverse 1516
ggggcttcgataccaacaaaaacatctgg

SEQ ID NO: 1527 - DNA, 29 bp, Artificial
  primer LPXB reverse 1517
ggggcttcgataccaacaaacacatcagg

SEQ ID NO: 1528 - DNA, 29 bp, Artificial
  primer LPXB reverse 1518
ggggcttcgataccaacaaacacatccgg

SEQ ID NO: 1529 - DNA, 29 bp, Artificial
  primer LPXB reverse 1519
ggggcttcgataccaacaaacacatctgg

SEQ ID NO: 1530 - DNA, 29 bp, Artificial
  primer LPXB reverse 1520
ggggcttcgataccaacaaacacatcggg

SEQ ID NO: 1531 - DNA, 29 bp, Artificial
  primer LPXB reverse 1521
ggggcttcgataccaacaaagacatcagg

SEQ ID NO: 1532 - DNA, 29 bp, Artificial
  primer LPXB reverse 1522
ggggcttcgataccaacaaagacatccgg

SEQ ID NO: 1533 - DNA, 29 bp, Artificial
  primer LPXB reverse 1523
ggggcttcgataccaacaaagacatcggg

SEQ ID NO: 1534 - DNA, 29 bp, Artificial
  primer LPXB reverse 1524
ggggcttcgataccaacaaagacatctgg

SEQ ID NO: 1535 - DNA, 29 bp, Artificial
  primer LPXB reverse 1525
ggggcttcgataccaacgaaaacatcagg

SEQ ID NO: 1536 - DNA, 29 bp, Artificial
  primer LPXB reverse 1526
ggggcttcgataccaacgaaaacatccgg

SEQ ID NO: 1537 - DNA, 29 bp, Artificial
  primer LPXB reverse 1527
ggggcttcgataccaacgaaaacatcggg

SEQ ID NO: 1538 - DNA, 29 bp, Artificial
  primer LPXB reverse 1528
ggggcttcgataccaacgaaaacatctgg

SEQ ID NO: 1539 - DNA, 29 bp, Artificial
  primer LPXB reverse 1529
ggggcttcgataccaacgaacacatcagg

SEQ ID NO: 1540 - DNA, 29 bp, Artificial
  primer LPXB reverse 1530
ggggcttcgataccaacgaacacatccgg

SEQ ID NO: 1541 - DNA, 29 bp, Artificial
  primer LPXB reverse 1531
ggggcttcgataccaacgaacacatctgg

SEQ ID NO: 1542 - DNA, 29 bp, Artificial
  primer LPXB reverse 1532
ggggcttcgataccaacgaacacatcggg

SEQ ID NO: 1543 - DNA, 29 bp, Artificial
  primer LPXB reverse 1533
ggggcttcgataccaacgaagacatcagg

SEQ ID NO: 1544 - DNA, 29 bp, Artificial
  primer LPXB reverse 1534
ggggcttcgataccaacgaagacatccgg

SEQ ID NO: 1545 - DNA, 29 bp, Artificial
  primer LPXB reverse 1535
ggggcttcgataccaacgaagacatcggg

SEQ ID NO: 1546 - DNA, 29 bp, Artificial
  primer LPXB reverse 1536
ggggcttcgataccaacgaagacatctgg

SEQ ID NO: 1547 - DNA, 29 bp, Artificial
  primer LPXB reverse 1537
ggggcttcgatacccacaaaaacatcagg

SEQ ID NO: 1548 - DNA, 29 bp, Artificial
  primer LPXB reverse 1538
ggggcttcgatacccacaaaaacatccgg

SEQ ID NO: 1549 - DNA, 29 bp, Artificial
  primer LPXB reverse 1539
ggggcttcgatacccacaaaaacatcggg

SEQ ID NO: 1550 - DNA, 29 bp, Artificial
  primer LPXB reverse 1540
ggggcttcgatacccacaaaaacatctgg

SEQ ID NO: 1551 - DNA, 29 bp, Artificial
  primer LPXB reverse 1541
ggggcttcgatacccacaaacacatcagg

SEQ ID NO: 1552 - DNA, 29 bp, Artificial
  primer LPXB reverse 1542
ggggcttcgatacccacaaacacatccgg

SEQ ID NO: 1553 - DNA, 29 bp, Artificial
  primer LPXB reverse 1543
ggggcttcgatacccacaaacacatctgg

SEQ ID NO: 1554 - DNA, 29 bp, Artificial
  primer LPXB reverse 1544
ggggcttcgatacccacaaacacatcggg

SEQ ID NO: 1555 - DNA, 29 bp, Artificial
  primer LPXB reverse 1545
ggggcttcgatacccacaaagacatcagg

SEQ ID NO: 1556 - DNA, 29 bp, Artificial
  primer LPXB reverse 1546
ggggcttcgatacccacaaagacatccgg

SEQ ID NO: 1557 - DNA, 29 bp, Artificial
  primer LPXB reverse 1547
ggggcttcgatacccacaaagacatcggg

SEQ ID NO: 1558 - DNA, 29 bp, Artificial
  primer LPXB reverse 1548
ggggcttcgatacccacaaagacatctgg

SEQ ID NO: 1559 - DNA, 29 bp, Artificial
  primer LPXB reverse 1549
ggggcttcgatacccacgaaaacatcagg

SEQ ID NO: 1560 - DNA, 29 bp, Artificial
  primer LPXB reverse 1550
ggggcttcgatacccacgaaaacatccgg

SEQ ID NO: 1561 - DNA, 29 bp, Artificial
  primer LPXB reverse 1551
ggggcttcgatacccacgaaaacatcggg

SEQ ID NO: 1562 - DNA, 29 bp, Artificial
  primer LPXB reverse 1552
ggggcttcgatacccacgaaaacatctgg

SEQ ID NO: 1563 - DNA, 29 bp, Artificial
  primer LPXB reverse 1553
ggggcttcgatacccacgaacacatcagg

SEQ ID NO: 1564 - DNA, 29 bp, Artificial
  primer LPXB reverse 1554
ggggcttcgatacccacgaacacatccgg

SEQ ID NO: 1565 - DNA, 29 bp, Artificial
  primer LPXB reverse 1555
ggggcttcgatacccacgaacacatctgg

SEQ ID NO: 1566 - DNA, 29 bp, Artificial
  primer LPXB reverse 1556
ggggcttcgatacccacgaacacatcggg

SEQ ID NO: 1567 - DNA, 29 bp, Artificial
  primer LPXB reverse 1557
ggggcttcgatacccacgaagacatcagg

SEQ ID NO: 1568 - DNA, 29 bp, Artificial
  primer LPXB reverse 1558
ggggcttcgatacccacgaagacatccgg

SEQ ID NO: 1569 - DNA, 29 bp, Artificial
  primer LPXB reverse 1559
ggggcttcgatacccacgaagacatcggg

SEQ ID NO: 1570 - DNA, 29 bp, Artificial
  primer LPXB reverse 1560
ggggcttcgatacccacgaagacatctgg

SEQ ID NO: 1571 - DNA, 29 bp, Artificial
  primer LPXB reverse 1561
ggggcttcgataccgacaaaaacatcagg

SEQ ID NO: 1572 - DNA, 29 bp, Artificial
  primer LPXB reverse 1562
ggggcttcgataccgacaaaaacatccgg

SEQ ID NO: 1573 - DNA, 29 bp, Artificial
  primer LPXB reverse 1563
ggggcttcgataccgacaaaaacatcggg

SEQ ID NO: 1574 - DNA, 29 bp, Artificial
  primer LPXB reverse 1564
ggggcttcgataccgacaaaaacatctgg

SEQ ID NO: 1575 - DNA, 29 bp, Artificial
  primer LPXB reverse 1565
ggggcttcgataccgacaaacacatcagg

SEQ ID NO: 1576 - DNA, 29 bp, Artificial
  primer LPXB reverse 1566
ggggcttcgataccgacaaacacatccgg

SEQ ID NO: 1577 - DNA, 29 bp, Artificial
  primer LPXB reverse 1567
ggggcttcgataccgacaaacacatctgg

SEQ ID NO: 1578 - DNA, 29 bp, Artificial
  primer LPXB reverse 1568
ggggcttcgataccgacaaacacatcggg

SEQ ID NO: 1579 - DNA, 29 bp, Artificial
  primer LPXB reverse 1569
ggggcttcgataccgacaaagacatcagg

SEQ ID NO: 1580 - DNA, 29 bp, Artificial
  primer LPXB reverse 1570
ggggcttcgataccgacaaagacatccgg

SEQ ID NO: 1581 - DNA, 29 bp, Artificial
  primer LPXB reverse 1571
ggggcttcgataccgacaaagacatcggg

SEQ ID NO: 1582 - DNA, 29 bp, Artificial
  primer LPXB reverse 1572
ggggcttcgataccgacaaagacatctgg

SEQ ID NO: 1583 - DNA, 29 bp, Artificial
  primer LPXB reverse 1573
ggggcttcgataccgacgaaaacatcagg

SEQ ID NO: 1584 - DNA, 29 bp, Artificial
  primer LPXB reverse 1574
ggggcttcgataccgacgaaaacatccgg

SEQ ID NO: 1585 - DNA, 29 bp, Artificial
  primer LPXB reverse 1575
ggggcttcgataccgacgaaaacatcggg

SEQ ID NO: 1586 - DNA, 29 bp, Artificial
  primer LPXB reverse 1576
ggggcttcgataccgacgaaaacatctgg

SEQ ID NO: 1587 - DNA, 29 bp, Artificial
  primer LPXB reverse 1577
ggggcttcgataccgacgaacacatcagg

SEQ ID NO: 1588 - DNA, 29 bp, Artificial
  primer LPXB reverse 1578
ggggcttcgataccgacgaacacatccgg

SEQ ID NO: 1589 - DNA, 29 bp, Artificial
  primer LPXB reverse 1579
ggggcttcgataccgacgaacacatctgg

SEQ ID NO: 1590 - DNA, 29 bp, Artificial
  primer LPXB reverse 1580
ggggcttcgataccgacgaacacatcggg

SEQ ID NO: 1591 - DNA, 29 bp, Artificial
  primer LPXB reverse 1581
ggggcttcgataccgacgaagacatcagg

SEQ ID NO: 1592 - DNA, 29 bp, Artificial
  primer LPXB reverse 1582
ggggcttcgataccgacgaagacatccgg

SEQ ID NO: 1593 - DNA, 29 bp, Artificial
  primer LPXB reverse 1583
ggggcttcgataccgacgaagacatcggg

SEQ ID NO: 1594 - DNA, 29 bp, Artificial
  primer LPXB reverse 1584
ggggcttcgataccgacgaagacatctgg

SEQ ID NO: 1595 - DNA, 29 bp, Artificial
  primer LPXB reverse 1585
ggggcttcgatcccaacaaaaacatcagg

SEQ ID NO: 1596 - DNA, 29 bp, Artificial
  primer LPXB reverse 1586
ggggcttcgatcccaacaaaaacatccgg

SEQ ID NO: 1597 - DNA, 29 bp, Artificial
  primer LPXB reverse 1587
ggggcttcgatcccaacaaaaacatcggg

SEQ ID NO: 1598 - DNA, 29 bp, Artificial
  primer LPXB reverse 1588
ggggcttcgatcccaacaaaaacatctgg

SEQ ID NO: 1599 - DNA, 29 bp, Artificial
  primer LPXB reverse 1589
ggggcttcgatcccaacaaacacatcagg

SEQ ID NO: 1600 - DNA, 29 bp, Artificial
  primer LPXB reverse 1590
ggggcttcgatcccaacaaacacatccgg

SEQ ID NO: 1601 - DNA, 29 bp, Artificial
  primer LPXB reverse 1591
ggggcttcgatcccaacaaacacatctgg

SEQ ID NO: 1602 - DNA, 29 bp, Artificial
  primer LPXB reverse 1592
ggggcttcgatcccaacaaacacatcggg

SEQ ID NO: 1603 - DNA, 29 bp, Artificial
  primer LPXB reverse 1593
ggggcttcgatcccaacaaagacatcagg

SEQ ID NO: 1604 - DNA, 29 bp, Artificial
  primer LPXB reverse 1594
ggggcttcgatcccaacaaagacatccgg

SEQ ID NO: 1605 - DNA, 29 bp, Artificial
  primer LPXB reverse 1595
ggggcttcgatcccaacaaagacatcggg

SEQ ID NO: 1606 - DNA, 29 bp, Artificial
  primer LPXB reverse 1596
ggggcttcgatcccaacaaagacatctgg

SEQ ID NO: 1607 - DNA, 29 bp, Artificial
  primer LPXB reverse 1597
ggggcttcgatcccaacgaaaacatcagg

SEQ ID NO: 1608 - DNA, 29 bp, Artificial
  primer LPXB reverse 1598
ggggcttcgatcccaacgaaaacatccgg

SEQ ID NO: 1609 - DNA, 29 bp, Artificial
  primer LPXB reverse 1599
ggggcttcgatcccaacgaaaacatcggg

SEQ ID NO: 1610 - DNA, 29 bp, Artificial
  primer LPXB reverse 1600
ggggcttcgatcccaacgaaaacatctgg

SEQ ID NO: 1611 - DNA, 29 bp, Artificial
  primer LPXB reverse 1601
ggggcttcgatcccaacgaacacatcagg

SEQ ID NO: 1612 - DNA, 29 bp, Artificial
  primer LPXB reverse 1602
ggggcttcgatcccaacgaacacatccgg

SEQ ID NO: 1613 - DNA, 29 bp, Artificial
  primer LPXB reverse 1603
ggggcttcgatcccaacgaacacatctgg

SEQ ID NO: 1614 - DNA, 29 bp, Artificial
  primer LPXB reverse 1604
ggggcttcgatcccaacgaacacatcggg

SEQ ID NO: 1615 - DNA, 29 bp, Artificial
  primer LPXB reverse 1605
ggggcttcgatcccaacgaagacatcagg

SEQ ID NO: 1616 - DNA, 29 bp, Artificial
  primer LPXB reverse 1606
ggggcttcgatcccaacgaagacatccgg

SEQ ID NO: 1617 - DNA, 29 bp, Artificial
  primer LPXB reverse 1607
ggggcttcgatcccaacgaagacatcggg

SEQ ID NO: 1618 - DNA, 29 bp, Artificial
  primer LPXB reverse 1608
ggggcttcgatcccaacgaagacatctgg

SEQ ID NO: 1619 - DNA, 29 bp, Artificial
  primer LPXB reverse 1609
ggggcttcgatccccacaaaaacatcagg

SEQ ID NO: 1620 - DNA, 29 bp, Artificial
  primer LPXB reverse 1610
ggggcttcgatccccacaaaaacatccgg

SEQ ID NO: 1621 - DNA, 29 bp, Artificial
  primer LPXB reverse 1611
ggggcttcgatccccacaaaaacatcggg

SEQ ID NO: 1622 - DNA, 29 bp, Artificial
  primer LPXB reverse 1612
ggggcttcgatccccacaaaaacatctgg

SEQ ID NO: 1623 - DNA, 29 bp, Artificial
  primer LPXB reverse 1613
ggggcttcgatccccacaaacacatcagg

SEQ ID NO: 1624 - DNA, 29 bp, Artificial
  primer LPXB reverse 1614
ggggcttcgatccccacaaacacatccgg

SEQ ID NO: 1625 - DNA, 29 bp, Artificial
  primer LPXB reverse 1615
ggggcttcgatccccacaaacacatctgg

SEQ ID NO: 1626 - DNA, 29 bp, Artificial
  primer LPXB reverse 1616
ggggcttcgatccccacaaacacatcggg

SEQ ID NO: 1627 - DNA, 29 bp, Artificial
  primer LPXB reverse 1617
ggggcttcgatccccacaaagacatcagg

SEQ ID NO: 1628 - DNA, 29 bp, Artificial
  primer LPXB reverse 1618
ggggcttcgatccccacaaagacatccgg

SEQ ID NO: 1629 - DNA, 29 bp, Artificial
  primer LPXB reverse 1619
ggggcttcgatccccacaaagacatcggg

SEQ ID NO: 1630 - DNA, 29 bp, Artificial
  primer LPXB reverse 1620
ggggcttcgatccccacaaagacatctgg

SEQ ID NO: 1631 - DNA, 29 bp, Artificial
  primer LPXB reverse 1621
ggggcttcgatccccacgaaaacatcagg

SEQ ID NO: 1632 - DNA, 29 bp, Artificial
  primer LPXB reverse 1622
ggggcttcgatccccacgaaaacatccgg

SEQ ID NO: 1633 - DNA, 29 bp, Artificial
  primer LPXB reverse 1623
ggggcttcgatccccacgaaaacatcggg

SEQ ID NO: 1634 - DNA, 29 bp, Artificial
  primer LPXB reverse 1624
ggggcttcgatccccacgaaaacatctgg

SEQ ID NO: 1635 - DNA, 29 bp, Artificial
  primer LPXB reverse 1625
ggggcttcgatccccacgaacacatcagg

SEQ ID NO: 1636 - DNA, 29 bp, Artificial
  primer LPXB reverse 1626
ggggcttcgatccccacgaacacatccgg

SEQ ID NO: 1637 - DNA, 29 bp, Artificial
  primer LPXB reverse 1627
ggggcttcgatccccacgaacacatctgg

SEQ ID NO: 1638 - DNA, 29 bp, Artificial
  primer LPXB reverse 1628
ggggcttcgatccccacgaacacatcggg

SEQ ID NO: 1639 - DNA, 29 bp, Artificial
  primer LPXB reverse 1629
ggggcttcgatccccacgaagacatcagg

SEQ ID NO: 1640 - DNA, 29 bp, Artificial
  primer LPXB reverse 1630
ggggcttcgatccccacgaagacatccgg

SEQ ID NO: 1641 - DNA, 29 bp, Artificial
  primer LPXB reverse 1631
ggggcttcgatccccacgaagacatcggg

SEQ ID NO: 1642 - DNA, 29 bp, Artificial
  primer LPXB reverse 1632
ggggcttcgatccccacgaagacatctgg

SEQ ID NO: 1643 - DNA, 29 bp, Artificial
  primer LPXB reverse 1633
ggggcttcgatcccgacaaaaacatcagg

SEQ ID NO: 1644 - DNA, 29 bp, Artificial
  primer LPXB reverse 1634
ggggcttcgatcccgacaaaaacatccgg

SEQ ID NO: 1645 - DNA, 29 bp, Artificial
  primer LPXB reverse 1635
ggggcttcgatcccgacaaaaacatcggg

SEQ ID NO: 1646 - DNA, 29 bp, Artificial
  primer LPXB reverse 1636
ggggcttcgatcccgacaaaaacatctgg

SEQ ID NO: 1647 - DNA, 29 bp, Artificial
  primer LPXB reverse 1637
ggggcttcgatcccgacaaacacatcagg

SEQ ID NO: 1648 - DNA, 29 bp, Artificial
  primer LPXB reverse 1638
ggggcttcgatcccgacaaacacatccgg

SEQ ID NO: 1649 - DNA, 29 bp, Artificial
  primer LPXB reverse 1639
ggggcttcgatcccgacaaacacatctgg

SEQ ID NO: 1650 - DNA, 29 bp, Artificial
  primer LPXB reverse 1640
ggggcttcgatcccgacaaacacatcggg

SEQ ID NO: 1651 - DNA, 29 bp, Artificial
  primer LPXB reverse 1641
ggggcttcgatcccgacaaagacatcagg

SEQ ID NO: 1652 - DNA, 29 bp, Artificial
  primer LPXB reverse 1642
ggggcttcgatcccgacaaagacatccgg

SEQ ID NO: 1653 - DNA, 29 bp, Artificial
  primer LPXB reverse 1643
ggggcttcgatcccgacaaagacatcggg

SEQ ID NO: 1654 - DNA, 29 bp, Artificial
  primer LPXB reverse 1644
ggggcttcgatcccgacaaagacatctgg

SEQ ID NO: 1655 - DNA, 29 bp, Artificial
  primer LPXB reverse 1645
ggggcttcgatcccgacgaaaacatcagg

SEQ ID NO: 1656 - DNA, 29 bp, Artificial
  primer LPXB reverse 1646
ggggcttcgatcccgacgaaaacatccgg

SEQ ID NO: 1657 - DNA, 29 bp, Artificial
  primer LPXB reverse 1647
ggggcttcgatcccgacgaaaacatcggg

SEQ ID NO: 1658 - DNA, 29 bp, Artificial
  primer LPXB reverse 1648
ggggcttcgatcccgacgaaaacatctgg

SEQ ID NO: 1659 - DNA, 29 bp, Artificial
  primer LPXB reverse 1649
ggggcttcgatcccgacgaacacatcagg

SEQ ID NO: 1660 - DNA, 29 bp, Artificial
  primer LPXB reverse 1650
ggggcttcgatcccgacgaacacatccgg

SEQ ID NO: 1661 - DNA, 29 bp, Artificial
  primer LPXB reverse 1651
ggggcttcgatcccgacgaacacatctgg

SEQ ID NO: 1662 - DNA, 29 bp, Artificial
  primer LPXB reverse 1652
ggggcttcgatcccgacgaacacatcggg

SEQ ID NO: 1663 - DNA, 29 bp, Artificial
  primer LPXB reverse 1653
ggggcttcgatcccgacgaagacatcagg

SEQ ID NO: 1664 - DNA, 29 bp, Artificial
  primer LPXB reverse 1654
ggggcttcgatcccgacgaagacatccgg

SEQ ID NO: 1665 - DNA, 29 bp, Artificial
  primer LPXB reverse 1655
ggggcttcgatcccgacgaagacatcggg

SEQ ID NO: 1666 - DNA, 29 bp, Artificial
  primer LPXB reverse 1656
ggggcttcgatcccgacgaagacatctgg

SEQ ID NO: 1667 - DNA, 29 bp, Artificial
  primer LPXB reverse 1657
ggggcttcgattccaacaaaaacatcagg

SEQ ID NO: 1668 - DNA, 29 bp, Artificial
  primer LPXB reverse 1658
ggggcttcgattccaacaaaaacatccgg

SEQ ID NO: 1669 - DNA, 29 bp, Artificial
  primer LPXB reverse 1659
ggggcttcgattccaacaaaaacatcggg

SEQ ID NO: 1670 - DNA, 29 bp, Artificial
  primer LPXB reverse 1660
ggggcttcgattccaacaaaaacatctgg

SEQ ID NO: 1671 - DNA, 29 bp, Artificial
  primer LPXB reverse 1661
ggggcttcgattccaacaaacacatcagg

SEQ ID NO: 1672 - DNA, 29 bp, Artificial
  primer LPXB reverse 1662
ggggcttcgattccaacaaacacatccgg

SEQ ID NO: 1673 - DNA, 29 bp, Artificial
  primer LPXB reverse 1663
ggggcttcgattccaacaaacacatctgg

SEQ ID NO: 1674 - DNA, 29 bp, Artificial
  primer LPXB reverse 1664
ggggcttcgattccaacaaacacatcggg

SEQ ID NO: 1675 - DNA, 29 bp, Artificial
  primer LPXB reverse 1665
ggggcttcgattccaacaaagacatcagg

SEQ ID NO: 1676 - DNA, 29 bp, Artificial
  primer LPXB reverse 1666
ggggcttcgattccaacaaagacatccgg

SEQ ID NO: 1677 - DNA, 29 bp, Artificial
  primer LPXB reverse 1667
ggggcttcgattccaacaaagacatcggg

SEQ ID NO: 1678 - DNA, 29 bp, Artificial
  primer LPXB reverse 1668
ggggcttcgattccaacaaagacatctgg

SEQ ID NO: 1679 - DNA, 29 bp, Artificial
  primer LPXB reverse 1669
ggggcttcgattccaacgaaaacatcagg

SEQ ID NO: 1680 - DNA, 29 bp, Artificial
  primer LPXB reverse 1670
ggggcttcgattccaacgaaaacatccgg

SEQ ID NO: 1681 - DNA, 29 bp, Artificial
  primer LPXB reverse 1671
ggggcttcgattccaacgaaaacatcggg

SEQ ID NO: 1682 - DNA, 29 bp, Artificial
  primer LPXB reverse 1672
ggggcttcgattccaacgaaaacatctgg

SEQ ID NO: 1683 - DNA, 29 bp, Artificial
  primer LPXB reverse 1673
ggggcttcgattccaacgaacacatcagg

SEQ ID NO: 1684 - DNA, 29 bp, Artificial
  primer LPXB reverse 1674
ggggcttcgattccaacgaacacatccgg

SEQ ID NO: 1685 - DNA, 29 bp, Artificial
  primer LPXB reverse 1675
ggggcttcgattccaacgaacacatctgg

SEQ ID NO: 1686 - DNA, 29 bp, Artificial
  primer LPXB reverse 1676
ggggcttcgattccaacgaacacatcggg

SEQ ID NO: 1687 - DNA, 29 bp, Artificial
  primer LPXB reverse 1677
ggggcttcgattccaacgaagacatcagg

SEQ ID NO: 1688 - DNA, 29 bp, Artificial
  primer LPXB reverse 1678
ggggcttcgattccaacgaagacatccgg

SEQ ID NO: 1689 - DNA, 29 bp, Artificial
  primer LPXB reverse 1679
ggggcttcgattccaacgaagacatcggg

SEQ ID NO: 1690 - DNA, 29 bp, Artificial
  primer LPXB reverse 1680
ggggcttcgattccaacgaagacatctgg

SEQ ID NO: 1691 - DNA, 29 bp, Artificial
  primer LPXB reverse 1681
ggggcttcgattcccacaaaaacatcagg

SEQ ID NO: 1692 - DNA, 29 bp, Artificial
  primer LPXB reverse 1682
ggggcttcgattcccacaaaaacatccgg

SEQ ID NO: 1693 - DNA, 29 bp, Artificial
  primer LPXB reverse 1683
ggggcttcgattcccacaaaaacatcggg

SEQ ID NO: 1694 - DNA, 29 bp, Artificial
  primer LPXB reverse 1684
ggggcttcgattcccacaaaaacatctgg

SEQ ID NO: 1695 - DNA, 29 bp, Artificial
  primer LPXB reverse 1685
ggggcttcgattcccacaaacacatcagg

SEQ ID NO: 1696 - DNA, 29 bp, Artificial
  primer LPXB reverse 1686
ggggcttcgattcccacaaacacatccgg

SEQ ID NO: 1697 - DNA, 29 bp, Artificial
  primer LPXB reverse 1687
ggggcttcgattcccacaaacacatctgg

SEQ ID NO: 1698 - DNA, 29 bp, Artificial
  primer LPXB reverse 1688
ggggcttcgattcccacaaacacatcggg

SEQ ID NO: 1699 - DNA, 29 bp, Artificial
  primer LPXB reverse 1689
ggggcttcgattcccacaaagacatcagg

SEQ ID NO: 1700 - DNA, 29 bp, Artificial
  primer LPXB reverse 1690
ggggcttcgattcccacaaagacatccgg

SEQ ID NO: 1701 - DNA, 29 bp, Artificial
  primer LPXB reverse 1691
ggggcttcgattcccacaaagacatcggg

SEQ ID NO: 1702 - DNA, 29 bp, Artificial
  primer LPXB reverse 1692
ggggcttcgattcccacaaagacatctgg

SEQ ID NO: 1703 - DNA, 29 bp, Artificial
  primer LPXB reverse 1693
ggggcttcgattcccacgaaaacatcagg

SEQ ID NO: 1704 - DNA, 29 bp, Artificial
  primer LPXB reverse 1694
ggggcttcgattcccacgaaaacatccgg

SEQ ID NO: 1705 - DNA, 29 bp, Artificial
  primer LPXB reverse 1695
ggggcttcgattcccacgaaaacatcggg

SEQ ID NO: 1706 - DNA, 29 bp, Artificial
  primer LPXB reverse 1696
ggggcttcgattcccacgaaaacatctgg

SEQ ID NO: 1707 - DNA, 29 bp, Artificial
  primer LPXB reverse 1697
ggggcttcgattcccacgaacacatcagg

SEQ ID NO: 1708 - DNA, 29 bp, Artificial
  primer LPXB reverse 1698
ggggcttcgattcccacgaacacatccgg

SEQ ID NO: 1709 - DNA, 29 bp, Artificial
  primer LPXB reverse 1699
ggggcttcgattcccacgaacacatctgg

SEQ ID NO: 1710 - DNA, 29 bp, Artificial
  primer LPXB reverse 1700
ggggcttcgattcccacgaacacatcggg

SEQ ID NO: 1711 - DNA, 29 bp, Artificial
  primer LPXB reverse 1701
ggggcttcgattcccacgaagacatcagg

SEQ ID NO: 1712 - DNA, 29 bp, Artificial
  primer LPXB reverse 1702
ggggcttcgattcccacgaagacatccgg

SEQ ID NO: 1713 - DNA, 29 bp, Artificial
  primer LPXB reverse 1703
ggggcttcgattcccacgaagacatcggg

SEQ ID NO: 1714 - DNA, 29 bp, Artificial
  primer LPXB reverse 1704
ggggcttcgattcccacgaagacatctgg

SEQ ID NO: 1715 - DNA, 29 bp, Artificial
  primer LPXB reverse 1705
ggggcttcgattccgacaaaaacatcagg

SEQ ID NO: 1716 - DNA, 29 bp, Artificial
  primer LPXB reverse 1706
ggggcttcgattccgacaaaaacatccgg

SEQ ID NO: 1717 - DNA, 29 bp, Artificial
  primer LPXB reverse 1707
ggggcttcgattccgacaaaaacatcggg

SEQ ID NO: 1718 - DNA, 29 bp, Artificial
  primer LPXB reverse 1708
ggggcttcgattccgacaaaaacatctgg

SEQ ID NO: 1719 - DNA, 29 bp, Artificial
  primer LPXB reverse 1709
ggggcttcgattccgacaaacacatcagg

SEQ ID NO: 1720 - DNA, 29 bp, Artificial
  primer LPXB reverse 1710
ggggcttcgattccgacaaacacatccgg

SEQ ID NO: 1721 - DNA, 29 bp, Artificial
  primer LPXB reverse 1711
ggggcttcgattccgacaaacacatctgg

SEQ ID NO: 1722 - DNA, 29 bp, Artificial
  primer LPXB reverse 1712
ggggcttcgattccgacaaacacatcggg

SEQ ID NO: 1723 - DNA, 29 bp, Artificial
  primer LPXB reverse 1713
ggggcttcgattccgacaaagacatcagg

SEQ ID NO: 1724 - DNA, 29 bp, Artificial
  primer LPXB reverse 1714
ggggcttcgattccgacaaagacatccgg

SEQ ID NO: 1725 - DNA, 29 bp, Artificial
  primer LPXB reverse 1715
ggggcttcgattccgacaaagacatcggg

SEQ ID NO: 1726 - DNA, 29 bp, Artificial
  primer LPXB reverse 1716
ggggcttcgattccgacaaagacatctgg

SEQ ID NO: 1727 - DNA, 29 bp, Artificial
  primer LPXB reverse 1717
ggggcttcgattccgacgaaaacatcagg

SEQ ID NO: 1728 - DNA, 29 bp, Artificial
  primer LPXB reverse 1718
ggggcttcgattccgacgaaaacatccgg

SEQ ID NO: 1729 - DNA, 29 bp, Artificial
  primer LPXB reverse 1719
ggggcttcgattccgacgaaaacatcggg

SEQ ID NO: 1730 - DNA, 29 bp, Artificial
  primer LPXB reverse 1720
ggggcttcgattccgacgaaaacatctgg

SEQ ID NO: 1731 - DNA, 29 bp, Artificial
  primer LPXB reverse 1721
ggggcttcgattccgacgaacacatcagg

SEQ ID NO: 1732 - DNA, 29 bp, Artificial
  primer LPXB reverse 1722
ggggcttcgattccgacgaacacatccgg

SEQ ID NO: 1733 - DNA, 29 bp, Artificial
  primer LPXB reverse 1723
ggggcttcgattccgacgaacacatctgg

SEQ ID NO: 1734 - DNA, 29 bp, Artificial
  primer LPXB reverse 1724
ggggcttcgattccgacgaacacatcggg

SEQ ID NO: 1735 - DNA, 29 bp, Artificial
  primer LPXB reverse 1725
ggggcttcgattccgacgaagacatcagg

SEQ ID NO: 1736 - DNA, 29 bp, Artificial
  primer LPXB reverse 1726
ggggcttcgattccgacgaagacatccgg

SEQ ID NO: 1737 - DNA, 29 bp, Artificial
  primer LPXB reverse 1727
ggggcttcgattccgacgaagacatcggg

SEQ ID NO: 1738 - DNA, 29 bp, Artificial
  primer LPXB reverse 1728
ggggcttcgattccgacgaagacatctgg

SEQ ID NO: 1739 - DNA, 17 bp, Artificial
  primer HDA1 forward
cctacgggaggcagcag

SEQ ID NO: 1740 - DNA, 17 bp, Artificial
  primer HDA2 reverse
attaccgcggctgctgg

SEQ ID NO: 1741 - DNA, 29 bp, Artificial
  primer LPXA forward
gtgattgayaaawccgcctttrtbcatcc

SEQ ID NO: 1742 - DNA, 20 bp, Artificial
  primer LPXA reverse
asatcytgrtthrcttcrcc

SEQ ID NO: 1743 - DNA, 29 bp, Artificial
  primer E. coli LPXA-forward
gtgattgataaatccgcctttgtgcatcc

SEQ ID NO: 1744 - DNA, 20 bp, Artificial
  primer E.coli LPXA-reverse
agatcctggttaacttcgcc

SEQ ID NO: 1745 - DNA, 1542 bp, Escherichia coli
aaattgaagagtttgatcatggctcagattgaacgctggcggcaggcctaacacatgcaagtcgaacggtaacaggaagcagcttgctgctttgctgacgagtggcggacgggtgagtaatgtctgggaaactgcccgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggagccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 1746 - DNA, 1148 bp, Escherichia coli
atgactgaacagcgtccattaacgattgccctggtcgccggagaaacctccggcgatatcctgggggccggtttaatccgcgctctgaaagaacgtgtgcccaacgcccgctttgttggtgttgccgggccacgaatgcaggctgaaggctgcgaagcctggtacgaaatggaagaactggcagtgatgggcattgttgaagtgctcggtcgtctgcgtcgcttactacatattcgtgccgatctgacaaagcgttttggcgaactgaagccagatgtttttgttggtattgatgcgcctgacttcaatattactcttgaaggtaacctcaaaaagcagggtatcaaaaccattcattacgtcagtccgtccgtctgggcgtggcgacagaaacgcgttttcaaaataggcagagccaccgatctggtgctcgcatttctgcctttcgaaaaagcgttttatgacaaatacaacgtaccgtgccgctttatcggtcataccatggctgatgccatgccattagatccagataaaaatggtgcccgtgatgtgctggggatccctcacgatgcccactgtctggcattgttgccgggcagccgtggtgcggaagtcgaaatgcttagcgccgattttctgaaaacggcccagcttttgcgccagacatatccggatctcgaaatcgtggtgccgctggtgaatgccaaacgccgcgagcagtttgaacgcatcaaagctgaagtcgcgccagacctgtcggttcatttgctggatggaatgggccgtgaggcgatggtcgccagcgatgcggccctactggcgtcggggacggcagccctggagtgtatgctggcgaaatgcccgatggtggtgggatatcgcatgaagccttttaccttctggttggcgaagcggctggtgaaaactgattatgtctcgctgccaaatctgctggcgggcagagagttagtcaaagagttattgcaggaagagtgtgagccgcaaaaactggctgcggcgctgttaccgctgttggcgaacgggaaaaccagccacgcgatgcacgataccttccgtgaactgcatcagcagatccgctgcaatgccgatgagcaggcggcacaagccgttctggagttacacaatga

SEQ ID NO: 1747 - DNA, 29 bp, Artificial
  E.coli LPXB-reverse primer
ggcgcatcaataccaacaaaaacatctgg

SEQ ID NO: 1748 - DNA, 21 bp, Artificial Sequence
  primer HDA1-F
actcctacgggaggcagcagt

SEQ ID NO: 1749 - DNA, 22 bp, Artificial Sequence
  primer HDA2-R
gtattaccgcggctgctggcac

SEQ ID NO: 1750 - DNA, 19 bp, Artificial Sequence
  primer EUBAC-F
tcctacgggaggcagcagt

SEQ ID NO: 1751 - DNA, 26 bp, Artificial Sequence
  primer EUBAC-R
ggactaccagggtatctaatcctgtt